(19) United States
(12) Patent Application Publication
(10) Pub. No.: US 2007/0259033 A1
(43) Pub. Date: Nov. 8, 2007

(54) Controlled release formulations exhibiting an ascending rate of release

(76) Inventor: Evangeline Cruz, Hayward, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/949,687

(22) Filed: Sep. 24, 2004

Related U.S. Application Data
(60) Provisional application No. 60/506,195, filed on Sep. 26, 2003.
(60) Provisional application No. 60/570,981, filed on May 14, 2004.

(51) Int. Cl. A61K 9/66; A61K 9/24

(52) U.S. Cl. 424/452; 424/473

(57) ABSTRACT

A sustained release dosage form is comprising a pharmaceutically active agent and pharmaceutically acceptable salts thereof and adapted to release as an erodible solid over a prolonged period of time, wherein the dosage form provides an ascending rate of release of the pharmaceutically active agent for at least about 4 hours. The dosage form is able to deliver high doses of poorly soluble or slowly dissolving active agents. When additional pharmaceutically active agents are present, the agents are released from the dosage form at rates that are proportional to the respective weights of each active agent in the dosage form. Methods of using the dosage forms to treat disease or conditions in human patients are also disclosed.

### CROSS-REFERENCE TO RELATED APPLICATION

This application claims the benefit of provisional application U.S. Serial No. 60/506,195, filed Sep. 26, 2003 and 60/570,981, filed May 14, 2004, which are incorporated by reference herein.

### FIELD OF THE INVENTION

This invention relates generally to solid dosage forms for administering pharmaceutical agents, methods of preparing the dosage forms, and methods of providing therapeutic agents to patients in need thereof, and the like.

### BACKGROUND OF THE INVENTION

Oral dosage forms for providing sustained release of pharmaceutically active agents are known in the art. These dosage forms are typically intended to provide a zero order rate of release of active agents for periods of time ranging from a few hours up to a day or more, with the goal of maintaining therapeutic levels in patients within a narrow range depending on the minimum effective concentrations of the drugs. However, certain drugs must be administered at high dosage, sometimes several times per day, to achieve a desired therapeutic effect. High dosages may require drug loading in drug compositions of the dosage forms to be as much as 90% or more of the overall weight of the composition. Such high loading requirements present problems in formulating compositions and fabricating dosage forms that are suitable for oral administration and can be swallowed without undue difficulty. High drug loadings present even greater problems when formulating dosage forms that are to be administered a limited number of times per day, such as for once-a-day dosing, because of the large unit dosage form required. While large daily doses of drug may be administered by multiple dosing throughout the day, multiple dosing regimens are often not preferred because of patient compliance problems, potential side effects and the dangers of overdosing. Accordingly, drug formulators have attempted to prepare formulations suitable for once-a-day or twice-a-day dosing regimens when possible, even where large doses of drug are required to be delivered over a prolonged period, for example 12 hours to 24 hours.

In addition, there are challenges to providing a particular delivery profile which is adequate to provide the necessary concentrations of drugs in patients when the drugs are metabolized or neutralized quickly, or where tolerance develops. The ability to deliver active agent at an ascending rate of release is one method of maintaining and controlling the concentrations of drugs in the plasma of patients. Recently, dosage forms have been disclosed for delivering certain drugs at approximately ascending rates of release such as ALZA Corporation's Concerta® methylphenidate product, and have been described in co-pending, commonly assigned U.S. Patent Application Publication No. 2001/0012847 to Lam, PCT Published Application Nos. U.S. Ser. No. 99/11920 (WO 9/62496); U.S. Ser. No. 97/13816 (WO 98/06380); and U.S. Ser. No. 97/16599 (WO 98/14168). Such disclosed dosage forms involve the use of multiple drug layers with sequentially increasing concentrations of drug in each drug layer, or a relatively large concentration (at least about 35%) of osmotically effective solute in the push layer, to produce the increasing delivery rate of drug over time. While such multi-layer tablet constructions represent a significant advancement to the art, these devices also have limited capability of delivering lowly soluble pharmaceutical agents, particularly those associated with relatively large doses of such agents, in a size that is acceptable for patients to swallow. The dosage forms developed to provide an ascending rate of release utilized bi-layer or tri-layer tablet cores, which provided a drug concentration gradient producing the ascending rate of release. A constant-release regimen was observed to have decreased clinical effectiveness compared to an immediate-release regimen at evaluation periods following administration of the second immediate-release dose, an effect likely due to the development of acute tolerance to the drug over the course of the day's treatment. On the other hand, an ascending-release regimen demonstrated comparable clinical efficacy to the immediate-release regimen during these evaluation periods. Thus, the ascending-release regimen provided using a drug concentration gradient avoided the decrease in therapeutic efficacy seen with the constant-release regimen due to the development of tolerance.

U.S. Pat. No. 6,245,357 describes osmotic dosage forms comprising a drug compartment and a pharmaceutically acceptable polymer hydrogel (maltodextrin, polyalkylene oxide, polyethylene oxide, carboxyalkylcellulose), contained within a bilayer interior wall and exterior wall and having a passageway, where the polymer exhibits an osmotic pressure gradient across the bilayer interior wall and exterior wall thereby imbibing fluid into the drug compartment to form a solution or a suspension comprising the drug that is hydrodynamically and osmotically delivered through a passageway from the dosage form. In certain embodiments, the dosage form further comprises a push displacement layer which expands to expel the drug from the dosage form. This patent describes that the interior wall of these dosage forms comprises a pore former which provides for increased permeability of the dosage form to water to compensate for the decrease in osmotic driving force that occurs as the osmagent and/or drug dissolves and is released from the dosage form. The dosage form was reported to exhibit a slow drug delivery until the osmotically-sensitive pore former dissolved or was leached from the inner wall. The eluted pore former caused the permeability of the inner wall to increase, which correspondingly caused the net permeability of the bilaminated inner wall-outer wall to increase over time. This increase in permeability was reported to offset any decrease in osmotic activity and produced a linear drug delivery profile. In addition, this patent describes dosage forms suitable for administering analgesic agents having a drug compartment comprising an opioid analgesic and a nonopioid analgesic and a polymer hydrogel, coated with an interior wall containing a pore former and an exterior wall.

Various devices and methods have been described having intended utility with respect to applications with high drug loading. For example, U.S. Pat. Nos. 4,892,778 and 4,940,465 describe dispensers for delivering a beneficial agent to an environment of use that include a semipermeable wall defining a compartment containing a layer of expandable material that pushes a drug layer out of the compartment formed by the wall. The exit orifice in the device is substantially the same diameter as the inner diameter of the compartment formed by the wall.

U.S. Pat. No. 6,368,626 describes high drug loading dosage forms for providing controlled release of active agents. This patent describes that the active agent is uniformly released from the dosage forms over a prolonged period of time, and that the release of the active agent from a dosage form does not vary positively or negatively by more than 30% from the mean rate of release of the active agent over a prolonged period of time, as determined in a USP Type 7 Interval Release Apparatus. This patent also points out that although high drug loading may be required in order to elicit a desired patient response, dosage forms which provide a uniform release rate of the active compound may allow the use of a lesser amount of compound per dosage form per day than would be calculated from simply multiplying the dose of active agent in the immediate release product by the number of times it is recommended to administer the immediate release product in a day. In addition, this patent describes high dosage levels in which the active compound is present from 40% to 90% by weight of the drug layer composition, but that preferably, the weight percent of active compound in the dosage forms of the invention is 75% or less, to allow for dosage forms that may be easily swallowed, and that in circumstances where it is desirable to administer an amount of drug that would exceed 75% of the drug layer composition, it is usually preferred to simultaneously administer two tablets or more of the dosage form with a total drug loading equal to the greater amount that would have been used in the single tablet.

However, there is still a need in the art for dosage forms capable of delivering drugs at an ascending release rate so as to provide sufficient drug to the patient in need thereof over time, to compensate for the development of tolerance, or to compensate for the rapid metabolism of the drug, and the like. There is a particular need for dosage forms that can deliver high doses of drugs, including poorly soluble and/or difficult to formulate drugs, at an ascending rate of release.

### SUMMARY OF THE INVENTION

Accordingly, it is a primary object of the invention to address the aforementioned need in the art by providing novel methods and dosage forms for delivering drugs at an ascending rate of release over a prolonged period of time.

Sustained release dosage forms are provided comprising a pharmaceutically active agent and pharmaceutically acceptable salts thereof and adapted to release as an erodible solid over a prolonged period of time, wherein the dosage form provides an ascending rate of release of the pharmaceutically active agent for at least about 4 hours. In preferred embodiments, the sustained release dosage form provides an ascending rate of release of the pharmaceutically active agent for from about 5 to about 8 to 10 hours, or in some instances, for longer periods of time. The sustained release dosage forms are useful for delivering active agents even when the pharmaceutically active agent is required to be administered to a patient in a high dose, or where the active agent has a low solubility and/or poor dissolution rate.

Preferably, the rate of release exhibited by the dosage form at its maximum rate of release is at least 20% greater than its minimum rate of release, typically the rate of release observed during the first hour or two after administration or initiation of dissolution testing. Typically the maximum rate of release occurs when about 70% of the active agent is being released. In other embodiments, the maximum rate of release exhibited by the dosage form is at least 40% greater than the minimum release rate exhibited by the dosage form. In additional embodiments, the maximum rate of release exhibited by the dosage form is at least 60% greater than the minimum release rate exhibited by the dosage form.

In certain embodiments, the erodible solid further comprises a binding agent, and a disintegrant, and it can include a surfactant and an osmagent. Preferred binding agents include polyoxyalkylenes, hydroxyalkylcelluloses, hydroxyalkylalkylcelluloses, and polyvinylpyrrolidones, and the like. Preferred disintegrants include croscarmellose sodium, crospovidone, sodium alginate, sodium starch glycolate, and the like.

Preferably, the sustained release dosage form provides an ascending rate of release of the pharmaceutically active agent until about 70% of the active agent has been released, and after the ascending rate of release, there is a rapid decrease in release rate. Preferably, the dosage form releases at least 90% of the active agent within about 12 hours.

In particular embodiments, the erodible solid further comprises a surfactant, which can be either a nonionic or ionic surfactant. Nonionic surfactants preferably include poloxamers, polyoxyethylene esters, sugar ester surfactants, sorbitan fatty acid esters, glycerol fatty acid esters, polyoxyethylene ethers of high molecular weight aliphatic alcohols, polyoxyethylene 40 sorbitol lanolin derivatives, polyoxyethylene 75 sorbitol lanolin derivatives, polyoxyethylene 20 sorbitol lanolin derivatives, polyoxyethylene 50 sorbitol lanolin derivatives, polyoxyethylene 6 sorbitol beeswax derivatives, polyoxyethylene 20 sorbitol beeswax derivatives, polyoxyethylene derivatives of fatty acid esters of sorbitan, and mixtures thereof. Preferred nonionic surfactants include poloxamers, fatty acid esters of polyoxyethylene, and sugar ester surfactants.

The sustained release dosage forms can deliver pharmaceutically active agents at an ascending rate of release at any drug loading. Preferably, the drug loading in the erodible solid is at least about 20% by weight and more preferably at least about 40% by weight.

In particular embodiments, the sustained release dosage forms are adapted to deliver high doses of active agent and to provide a high loading of the active agent. In certain embodiments, the pharmaceutically active agent is present in the erodible solid at a percent composition of at least 60 weight percent, and generally is present in the erodible solid in the range of from about 60 percent to about 95 percent by weight. In particular embodiments, the active agent is present in the erodible solid at a percent composition of from about 70 percent to about 90 percent by weight, or even at a drug loading of from about 75 percent to about 85 percent by weight. In certain embodiments, the erodible solid comprises from about 5 to about 15 percent by weight of a binding agent and a disintegrant. In additional embodiments, the erodible solid comprises from about 1 to about 15 percent by weight of a surfactant and can also contain an osmagent, typically less than 10 to 15 percent by weight.

In certain embodiments, the sustained release dosage form further comprises at least one additional pharmaceutically active agent in the erodible solid. The pharmaceutically active agents can have similar or different solubilities, and are released from the dosage form at rates that are proportional to the respective weights of each active agent in the dosage form.

The sustained release dosage form is useful for delivery of active agents that are poorly soluble. In a preferred embodiment, the pharmaceutically active agent typically has a solubility of less than about 50 mg/ml at 25° C., and may have a solubility of less than about 10 mg/ml at 25° C. In another preferred embodiment, the sustained release dosage form contains at least one additional pharmaceutically active agent, and at least one of the active agents has a solubility of less than about 50 mg/ml at 25° C.

In certain additional embodiments, the sustained release dosage form can further comprise an immediate release drug coating comprising an effective dose of at least one pharmaceutically active agent. Where additional active agents are present in the sustained release dosage form, the immediate release drug coating can also comprise the additional active agents. The immediate release drug coating acts to provide an immediate dose of active agents to a patient, and the sustained release dosage form provides a sustained release of active agent over the entire dosing interval, thereby providing a therapeutically effective dose of the active agents to a patient in need thereof.

In additional embodiments, the sustained release dosage form comprises: (1) a semipermeable wall defining a cavity and including an exit orifice formed or formable therein; (2) a drug layer comprising a therapeutically effective amount of a pharmaceutically active agent and pharmaceutically acceptable salts thereof contained within the cavity and located adjacent to the exit orifice; (3) a push displacement layer contained within the cavity and located distal from the exit orifice; (4) a flow-promoting layer in between the inner surface of the semipermeable wall and at least the external surface of the drug layer that is opposite the wall; and provides an ascending rate of release of the pharmaceutically active agent for at least about 4 hours. The drug layer is exposed to the environment of use as an erodible composition. More preferably, the dosage form provides an ascending rate of release of the pharmaceutically active agent for from about 5 to about 8 hours, or for 10 hours or more. In preferred embodiments, after the ascending rate of release, the dosage forms exhibit a rapid decrease in release rate. Preferably, the dosage form releases at least 90% of the active agent within about 12 hours.

Preferably, the dosage form provides an ascending rate of release of the pharmaceutically active agent until about 70 percent of the active agent has been released. Typically the minimum release rate is exhibited by the dosage form when less than about 10 to 20% of the active agent has been released. In particular embodiments, the maximum rate of release exhibited by the dosage form is at least 20% greater than the minimum release rate. In additional embodiments, the maximum rate of release exhibited by the dosage form is at least 40% greater than the minimum release rate. In yet other embodiments, the maximum rate of release exhibited by the dosage form is at least 60% greater than the minimum release rate exhibited by the dosage form.

The sustained release dosage forms are useful for delivering active agents even when the pharmaceutically active agent is required to be administered to a patient in a high dose, or where the active agent has a low solubility and/or poor dissolution rate.

In particular embodiments, the drug layer further comprises a binding agent, a disintegrant or mixtures thereof, and in certain other embodiments, the drug layer further comprises a surfactant and/or an osmagent. The surfactant can be a nonionic or ionic surfactant. Nonionic surfactants preferably include poloxamers, polyoxyethylene esters, sugar ester surfactants, sorbitan fatty acid esters, glycerol fatty acid esters, polyoxyethylene ethers of high molecular weight aliphatic alcohols, polyoxyethylene 40 sorbitol lanolin derivatives, polyoxyethylene 75 sorbitol lanolin derivatives, polyoxyethylene 20 sorbitol lanolin derivatives, polyoxyethylene 50 sorbitol lanolin derivatives, polyoxyethylene 6 sorbitol beeswax derivatives, polyoxyethylene 20 sorbitol beeswax derivatives, polyoxyethylene derivatives of fatty acid esters of sorbitan, and mixtures thereof. Preferred nonionic surfactants include poloxamers, a fatty acid esters of polyoxyethylene, and sugar ester surfactants.

The sustained release dosage forms can deliver pharmaceutically active agents at an ascending rate of release at any drug loading. Preferably, the drug layer contains at least about 20% active agent by weight and more preferably at least about 40% active agent by weight. In particular embodiments, the sustained release dosage forms are adapted to deliver high doses of active agent and to provide a high loading of the active agent. In certain embodiments, the pharmaceutically active agent is present in the drug layer at a percent composition of at least 60 weight percent, and generally is present in the drug layer in the range of from about 60 percent to about 95 percent by weight. In particular embodiments, the active agent is present in the drug layer at a percent composition of from about 70 percent to about 90 percent by weight, or even at a drug loading of from about 75 percent to about 85 percent by weight.

In certain embodiments, the sustained release dosage form further comprises at least one additional pharmaceutically active agent in the drug layer. The pharmaceutically active agents can have similar or different solubilities. In addition, the pharmaceutically active agents can be released from the dosage form at rates that are proportional to each other.

In certain additional embodiments, the sustained release dosage form can further comprise an immediate release drug coating comprising an effective dose of at least one pharmaceutically active agent, and where additional active agents are present in the sustained release dosage form, the immediate release drug coating can also comprise the additional active agents. The immediate release drug coating acts to provide an immediate dose of active agents to a patient, and the sustained release dosage form provides a sustained release of active agent over the entire dosing interval, thereby providing a therapeutically effective dose of the active agents to a patient in need thereof.

The pharmaceutically active agent can have any aqueous solubility. The sustained release dosage forms are particularly useful for delivering pharmaceutically active agents that are poorly soluble. Generally, the poorly soluble active agent has a solubility of less than about 50 mg/ml at 25° C, and may have a solubility of less than about 10 mg/ml at 25° C. The pharmaceutically active agent can be any pharmaceutically active agent, and in preferred embodiments is selected from a nonopioid analgesic agent, an antibiotic, an antiepileptic, or combinations thereof. In particular embodiments, at least one additional pharmaceutically active agent is included in the dosage form and can be selected from an opioid analgesic agent, a gastric protective agent, a 5-HT agonist, or other active agent.

The sustained release dosage forms can be used in methods for providing a sustained release of an increasing dose of a pharmaceutically active agent to a patient in need thereof. The sustained release dosage form is orally administered to a patient in need of treatment, and comprises a pharmaceutically active agent and pharmaceutically acceptable salts thereof adapted to release as an erodible solid over a prolonged period of time, and provides an ascending rate of release of the pharmaceutically active agent for at least about 4 hours.

In particular embodiments, methods for providing a sustained release of a therapeutically effective dose of a pharmaceutically active agent are provided, where the active agent is characterized by administration to a patient in a high dosage, low solubility and/or poor dissolution rate.

In additional embodiments, methods for providing a therapeutically effective dose of a pharmaceutically active agent to a patient in need thereof are provided, comprising orally administering a composition comprising a therapeutically effective amount of a pharmaceutically active agent present in a drug layer contained within a cavity defined by an at least partially semipermeable wall and having an exit means located adjacent thereto, a push displacement layer located within the cavity distal from the exit means providing a sustained release of the composition from the cavity when placed in an aqueous environment of use, and a flow-promoting layer located in between the inner surface of the semipermeable wall and at least the external surface of the drug layer that is opposite the wall, wherein the dosage form provides an ascending rate of release of the pharmaceutically active agent for at least about 4 hours. The method can further comprise utilizing a drug coating on the sustained release dosage form comprising a therapeutically effective amount of an immediate release therapeutic composition located on the outside surface of the at least partially semipermeable wall. The therapeutic composition preferably provides an ascending rate of release of the pharmaceutically active agent for from about 5 hours to about 8 hours or longer. In preferred embodiments, the drug layer comprises from about 60 to about 95% of the pharmaceutically active agent by weight, and more preferably from about 75 to about 85% of the pharmaceutically active agent by weight. In particular embodiments, the drug layer comprises from about 5 to about 15 percent by weight of a binding agent and a disintegrant, and optionally from about 1 to about 15 percent by weight of a surfactant.

In additional embodiments, methods for providing an effective concentration in the plasma of a patient of a pharmaceutically active agent that is metabolized relatively rapidly are provided, comprising orally administering a therapeutic composition comprising a pharmaceutically active agent and pharmaceutically acceptable salts thereof adapted to release as an erodible solid over a prolonged period of time, wherein the erodible solid comprises the pharmaceutically active agent, and wherein said therapeutic composition provides an ascending rate of release of the pharmaceutically active agent for at least about 4 hours. In preferred embodiments, the dosage form provides an ascending rate of release of the pharmaceutically active agent for from about 4 hours to about 8 hours.

The therapeutic composition can further comprise a drug coating comprising a therapeutically effective amount of the pharmaceutically active agent sufficient to provide an immediate effect in a patient in need thereof. In particular embodiments, the therapeutic composition provides a substantially zero order plasma profile of the pharmaceutically active agent in the patient. In additional embodiments, the therapeutic composition provides an ascending plasma profile of the pharmaceutically active agent in the patient. In certain other embodiments, the therapeutic composition provides a declining plasma profile of the pharmaceutically active agent in the patient. In a preferred embodiment, the dosage form comprises an immediate release drug coating that provides a therapeutically effective amount of the pharmaceutically active agent in the plasma of the patient and the ascending rate of release provided by the therapeutic composition maintains the concentration of the pharmaceutically active agent in the therapeutic range in the plasma of the patient for a prolonged period of time.

In yet other embodiments, methods are provided for providing an effective dose of a pharmaceutically active agent to which tolerance develops relatively rapidly in a patient, comprising orally administering a therapeutic composition comprising an effective dose of a pharmaceutically active agent to which tolerance develops relatively rapidly contained in a drug layer, an osmotic push composition, an at least partially semipermeable wall, and an exit means in the wall for delivering the therapeutic composition from the dosage form, and a flow-promoting layer located in between the inner surface of the semipermeable wall and at least the external surface of the drug layer that is opposite the wall, wherein said drug layer and push composition are surrounded by the at least partially semipermeable wall, wherein the drug layer is exposed to the environment of use as an erodible composition, and further wherein said dosage form provides an ascending rate of release of the pharmaceutically active agent thereby providing increasing concentrations of the pharmaceutically active agent in the plasma of the patient.

In a preferred embodiment, a method for treating pain in a human patient in need thereof is provided, comprising orally administering a dosage form comprising a therapeutic composition comprising a nonopioid analgesic, an opioid analgesic and pharmaceutically acceptable salts thereof adapted to release as an erodible solid over a prolonged period of time, wherein the nonopioid analgesic and the opioid analgesic are released at rates proportional to each other, and wherein the therapeutic composition provides an ascending rate of release of the nonopioid analgesic and the opioid analgesic for at least about 4 hours.

Additional objects, advantages and novel features of the invention will be set forth in part in the description which follows, and in part will become apparent to those skilled in the art upon examination of the following, or may be learned by practice of the invention.

### BRIEF DESCRIPTION OF THE DRAWINGS

FIG. 1 shows a schematic illustration of one embodiment of a dosage form according to the invention.

FIG. 2 illustrates the ascending release rate in vitro of acetaminophen and hydrocodone bitartrate from a representative dosage form and shows that the release rate of the two drugs is proportional.

FIGS. 3A and B illustrates the cumulative in vitro release rates of hydrocodone and acetaminophen, respectively, from several representative dosage forms having drug coatings providing immediate release in addition to sustained release.

FIG. 4A-D illustrates the in vitro release rates and cumulative release of acetaminophen and hydrocodone bitartrate from a representative dosage form having a T₆₀ of about 8 hours.

FIG. 5A-D illustrate the in vitro release rates and cumulative release of acetaminophen and hydrocodone bitartrate from a representative dosage form having a T₉₀ of about 6 hours.

FIG. 6A-D illustrate the in vitro release rates and cumulative release of acetaminophen and hydrocodone bitartrate from a representative dosage form having a T₉₀ of about 10 hours.

FIGS.7 A and B illustrate the cumulative in vitro release of acetaminophen and hydrocodone bitartrate from three representative dosage forms having T₉₀s of about 8 hours.

FIGS. 8A and B illustrate a comparison between the average in vivo plasma profiles of hydrocodone and acetaminophen, respectively, over a period of 48 hours obtained after a single administration of a representative dosage form and after administration of an immediate release dosage form dosed at zero, four and eight hours.

FIGS. 9A and B illustrate the release rate and cumulative in vitro release of ibuprofen from a representative dosage form containing ibuprofen and hydrocodone bitartrate.

FIG. 10 illustrates the in vitro release rate of ibuprofen from a representative dosage form containing ibuprofen and hydrocodone bitartrate.

### DETAILED DESCRIPTION OF THE INVENTION

Definitions and Overview

Before the present invention is described in detail, it is to be understood that unless otherwise indicated this invention is not limited to specific pharmaceutical agents, excipients, polymers, salts, or the like, as such may vary. It is also to be understood that the terminology used herein is for the purpose of describing particular embodiments only and is not intended to limit the scope of the present invention.

It must be noted that as used herein and in the claims, the singular forms “a,” “and” and “the” include plural referents unless the context clearly dictates otherwise. Thus, for example, reference to “a carrier” includes two or more carriers; reference to “a pharmaceutical agent” includes two or more pharmaceutical agents, and so forth.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

For clarity and convenience herein, the convention is utilized of designating the time of drug administration or initiation of dissolution testing as zero hours (t=0 hours) and times following administration in appropriate time units, e.g., t=30 minutes or t=2 hours, etc.

As used herein, the term “active agent” refers to a pharmaceutically active agent or a drug, and these terms may be used interchangeably.

As used herein, the phrase “ascending plasma profile” refers to an increase in the amount of a particular drug in the plasma of a patient over at least two sequential time intervals relative to the amount of the drug present in the plasma of the patient over the immediately preceding time interval. Generally, an ascending plasma profile will increase by at least about 10% over the time intervals exhibiting an ascending profile.

As used herein, the phrase “ascending release rate” refers to a dissolution rate that generally increases over time, such that the drug dissolves in the fluid at the environment of use at a rate that generally increases with time, rather than remaining constant or decreasing, until the dosage form is depleted of at least about 70% of the drug.

As used herein, the term “AUC” refers to the area under the concentration time curve, calculated using the trapezoidal rule and Clast/k, where Clast is the last observed concentration and k is the calculated elimination rate constant.

As used herein, the term “AUCt” refers to the area under the concentration time curve to last observed concentration calculated using the trapezoidal rule.

As used herein, the term “Cmax” refers to the plasma concentration of hydrocodone and/or acetaminophen at Tmax expressed as ng/mL and μg/mL, respectively, produced by the oral ingestion of a composition of the invention or the every four hour comparator (NORCO®). Unless specifically indicated, Cmax refers to the overall maximum observed concentration.

The terms “deliver” and “delivery” refer to separation of the pharmaceutical agent from the dosage form, wherein the pharmaceutical agent is able to dissolve in the fluid of the environment of use.

By “dosage form” is meant a pharmaceutical composition or device comprising an active pharmaceutical agent, or a pharmaceutically acceptable acid addition salt thereof, the composition or device optionally containing pharmacologically inactive ingredients, i.e., pharmaceutically acceptable excipients such as polymers, suspending agents, surfactants, disintegrants, dissolution modulating components, binders, diluents, lubricants, stabilizers, antioxidants, osmotic agents, colorants, plasticizers, coatings and the like, that are used to manufacture and deliver active pharmaceutical agents.

As used herein, the term “high dosage” refers to an active agent that is administered in a high dose to a patient. Typically a high dose is at least 100 mg per day, and can be up to 10,000 mg per day, or more.

As used herein, the term “immediate-release” refers to the substantially complete release of drug within a short time period following administration or initiation of dissolution testing, i.e., generally within a few minutes to about 1 hour.

As used herein, the phrase “in vivo/in vitro correlation” refers to the correspondence between release of drug from a dosage form as demonstrated by assays measuring the in vitro rate of release of drug from a dosage form and the delivery of drug from a dosage form in vivo as demonstrated by assays of residual drug in the dosage form after being administered orally.

As used herein, the phrase “low solubility and/or poor dissolution rate” refers an active agent that has a solubility of less than about 50 mg/ml, and preferably less than about 10 mg/ml, and that dissolves slowly relative to active agents that have a solubility greater than about 50 mg/ml.

As used herein, unless further specified, the term “a patient” means an individual patient and/or a population of patients in need of treatment for a disease or disorder.

By “pharmaceutically acceptable acid addition salt” or “pharmaceutically acceptable salt,” which are used interchangeably herein, are meant those salts in which the anion does not contribute significantly to the toxicity or pharmacological activity of the salt, and, as such, they are the pharmacological equivalent of the base form of the active agent. Examples of pharmaceutically acceptable acids that are useful for the purposes of salt formation include, but are not limited to, hydrochloric, hydrobromic, hydroiodic, sulfuric, citric, tartaric, methanesulfonic, fumaric, malic, maleic and mandelic acids. Pharmaceutically acceptable salts further include mucate, N-oxide, sulfate, acetate, phosphate dibasic, phosphate monobasic, acetate trihydrate, bi(heptafluorobutyrate), bi(methylcarbamate), bi(pentafluoropropionate), bi(pyridine-3-carboxylate), bi(trifluoroacetate), bitartrate, chlorhydrate, and sulfate pentahydrate, benzenesulfonate, benzoate, bicarbonate, bitartrate, bromide, calcium edetate, camsylate, carbonate, chloride, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methyinitrate, methylsulfate, mucate, napsylate, nitrate, pamoate (embonate), pantothenate, phosphate/diphosphate, polygalacturonate, salicylate, stearate, subacetate, succinate, sulfate, tannate, tartrate, teoclate, triethiodide, benzathine, chloroprocaine, choline, diethanolamine, ethylenediamine, meglumine, and procaine, aluminum, calcium, lithium, magnesium, potassium, sodium propionate, zinc, and the like.

As used herein, the term “proportional” (when referring to the release rate or delivery of the nonopioid analgesic and opioid analgesic from the dosage form) refers to the release or the rate of release of the two analgesic agents relative to each other, wherein the amount released is normalized to the total amount of each analgesic in the dosage form, i.e., the amount released is expressed as a percent of the total amount of each analgesic present in the dosage form. Generally, a proportional release rate of the nonopioid analgesic or of the opioid analgesic from the dosage form means that the relative release rate (expressed as percent release) or amount released (expressed as the cumulative release as a percent of the total amount present in the dosage form) of each drug is within about 20%, more preferably within about 10%, and most preferably within about 5% of the release rate or amount released of the other drug. In other words, at any point in time, the release rate of one agent (stated as a percentage of its total mount present in the dosage form) does not deviate more than about 20%, more preferably not more than about 10%, and most preferably not more than about 5% of the release rate of the other agent at the same point in time.

A drug “release rate” refers to the quantity of drug released from a dosage form per unit time, e.g., milligrams of drug released per hour (mg/hr). Drug release rates for drug dosage forms are typically measured as an in vitro rate of dissolution, i.e., a quantity of drug released from the dosage form per unit time measured under appropriate conditions and in a suitable fluid. For example, dissolution tests can be performed on dosage forms placed in metal coil sample holders attached to a USP Type VII bath indexer and immersed in about 50 ml of acidified water (pH=3) equilibrated in a constant temperature water bath at 37° C. Aliquots of the release rate solutions are tested to determine the amount of drug released from the dosage form, for example, the drug can be assayed or injected into a chromatographic system to quantify the amounts of drug released during the testing intervals.

Unless otherwise specified, a drug release rate obtained at a specified time following administration refers to the in vitro drug release rate obtained at the specified time following implementation of an appropriate dissolution test. The time at which a specified percentage of the drug within a dosage form has been released may be referenced as the “Tₓ” value, where “x” is the percent of drug that has been released. For example, a commonly used reference measurement for evaluating drug release from dosage forms is the time at which 90% of drug within the dosage form has been released. This measurement is referred to as the “T₉₀” for the dosage form.

As used herein, the term “sustained release” refers to the release of the drug from the dosage form over a period of many hours. Generally the sustained release occurs at such a rate that blood (e.g., plasma) concentrations in the patient administered the dosage form are maintained within the therapeutic range, that is, above the minimum effective analgesic concentration but below toxic levels, over a period of time of about 12 hours.

As used herein, the term “Tmax” refers to the time which elapses after administration of the dosage form at which the plasma concentration of hydrocodone and/or acetaminophen attains the maximum plasma concentrations.

As used herein, the phrase “zero order plasma profile” refers to a substantially flat or unchanging amount of a particular drug in the plasma of a patient over a particular time interval. Generally, a zero order plasma profile will vary by no more than about 30% from one time interval to the subsequent time interval, and preferably by no more than about 10% from one time interval to the next, and over the entire period of release, will show a substantially constant release rate and a flat curve of release rate versus time.

As used herein, the phrase “zero order release rate” refers to a substantially constant release rate, such that the drug dissolves in the fluid at the environment of use at a substantially constant rate. A zero order release rate can vary by as much as about 30% and preferably by no more than about 10% from the average release rate.

One skilled in the art will understand that therapeutic levels of a particular drug will vary according to many factors, including individual patient variability, health status such as renal and hepatic sufficiency, physical activity, the development of tolerance, inhibition of or the presence of alternative forms of cytochrome P450, and the nature of the disorder or disease.

It has been surprisingly discovered that the sustained release dosage forms of the present invention provide novel advantages that have not been achieved previously. The sustained release formulations surprisingly provide an ascending rate of release of the pharmaceutically active agents from the dosage form for at least about 4 hours. The sustained release dosage forms provide release of the active agents at ascending release rates, providing a unique ability to tailor the plasma concentration in the patient to either parallel plasma concentrations or differing plasma concentrations, such as would occur if one agent is metabolized at a slower rate than the other active agent. The active agents can be released from the dosage form at proportional rates of release. The active agents can be chosen so that their rates of inactivation or excretion are similar, thus providing a parallel plasma profile, or so that their rates of inactivation or excretion are different, thus providing a plasma profile that diverges.

In addition, in the event that tolerance or desensitization to a particular active agent occurs, an ascending release rate provides a means of overcoming the difficulty in maintaining effective therapeutic levels of the active agent. Thus, for any decrease in efficacy due to the development of tolerance, the increasing plasma concentrations provide a means for compensating for any reduced efficacy of the active agent, even under circumstances where target receptors in the patient have become less sensitive to the active agent.

Further, the disclosed formulations can provide a high loading of a relatively insoluble active agent and further provide possible synergistic or therapeutic combinations with additional active agents, having a similar or quite different solubility. The dosage forms can exhibit proportional delivery of both active agents (e.g., hydrocodone and acetaminophen or ibuprofen) even though the physical properties of the active agents (e.g., their solubilities), differ markedly from each other. The formulations can be administered to a human patient in a manner to provide effective concentrations of active agents relatively quickly and to further provide a sustained release to maintain levels of active agents sufficient to treat the condition or disorder for up to about 12 hours.

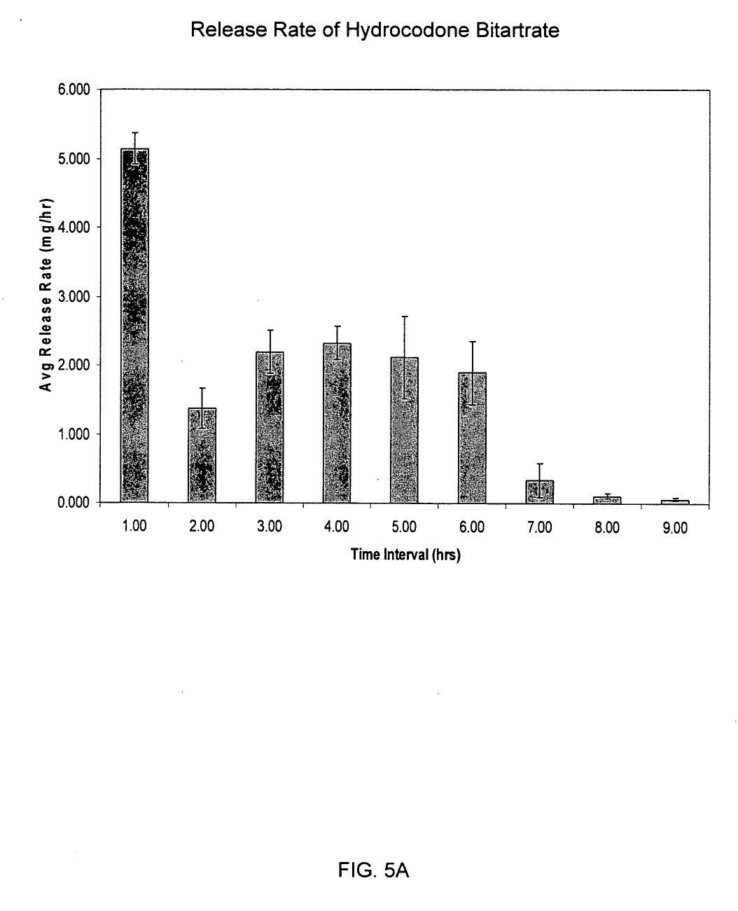

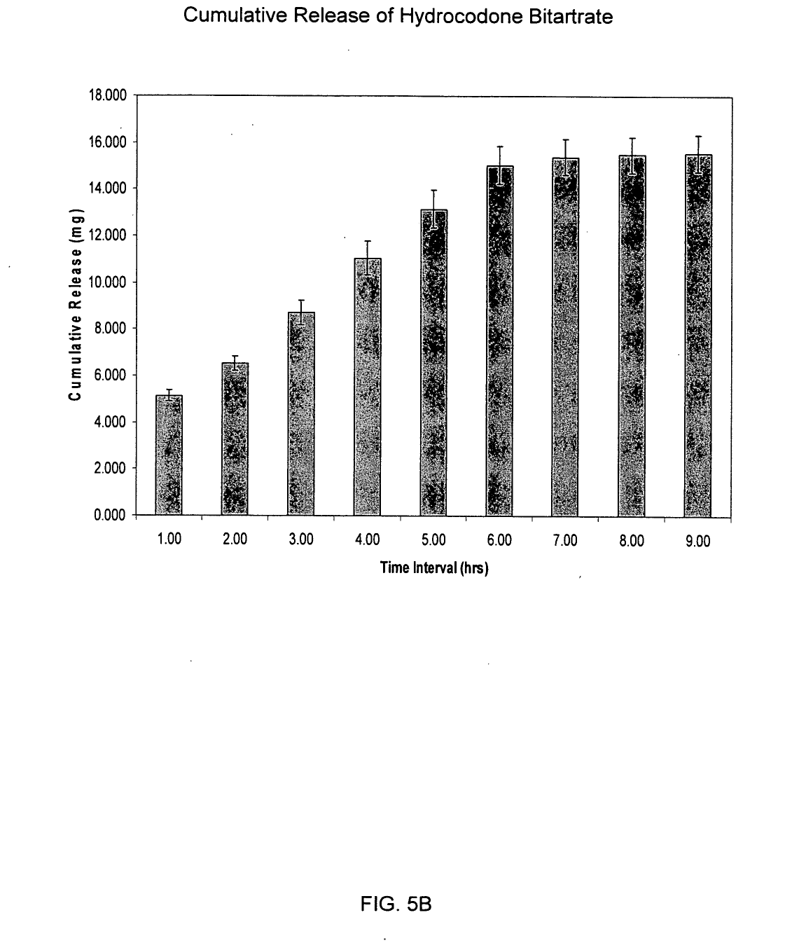

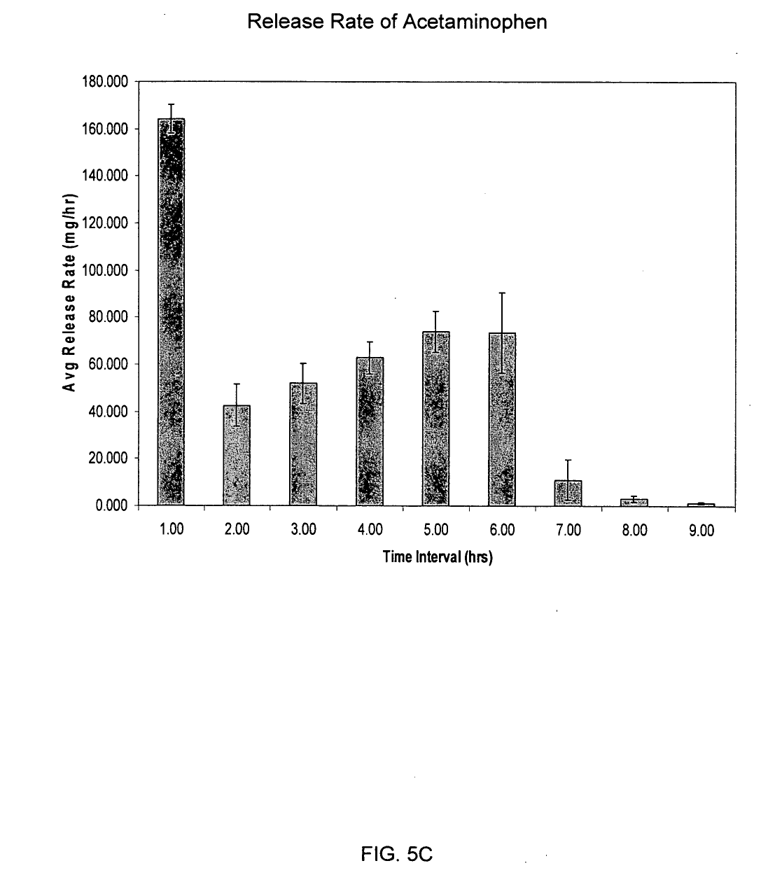

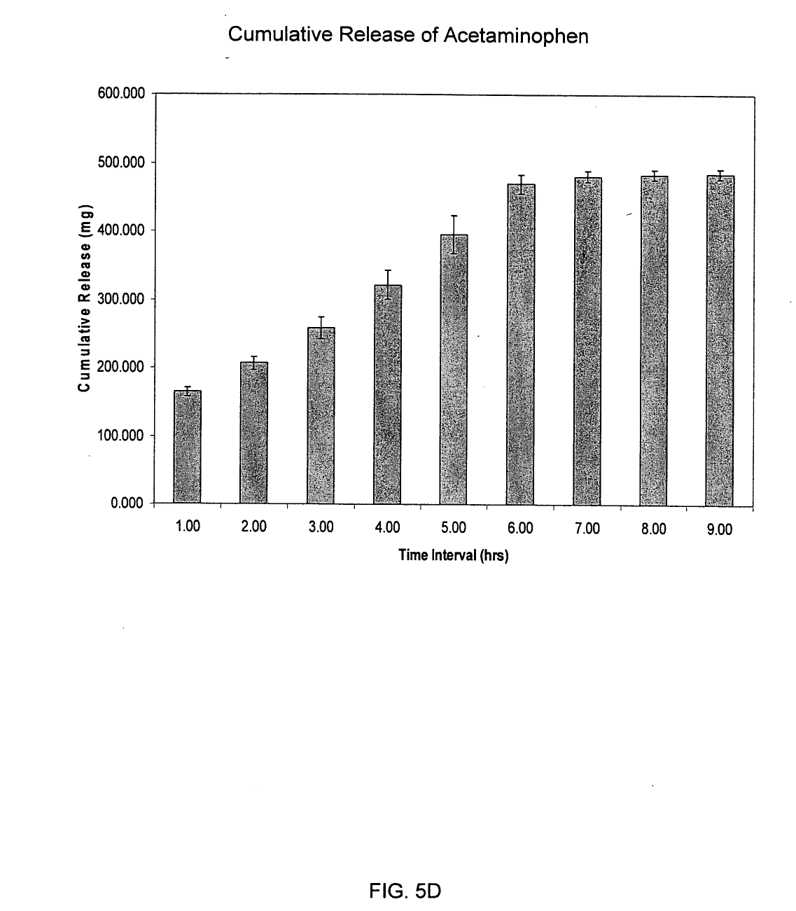

The release profiles provided show a close parallel between the amount of active agent in the drug coating (if any) and the sustained release portion of the dosage form and their release profiles from the dosage form, in that the amount released within one hour closely parallels the amount intended to be released immediately into the environment of use, while the amount released in a sustained release profile parallels the amount intended to be released over a prolonged period of time. For example, FIG. 5A shows the dissolution profile of a preferred embodiment, and shows that hydrocodone bitartrate is released at a rate of approximately 5 mg/hr during the first hour of dissolution testing, which closely parallels the amount incorporated into the immediate release drug coating and intended to be released within the first hour of administration. FIG. 5C shows that acetaminophen is released at a rate of approximately 163 mg/hr during the first hour of dissolution testing, which closely parallels the amount incorporated into the immediate release drug coating and intended to be released within the first hour of administration. FIGS. 5B and D show that essentially complete release of the active agent occurred over the period of dissolution testing.

The formulations also show proportional release of the active agents relative to one another, when more than one active agent is present. For example, as shown in Tables 3 and 4 in Example 4 below, the cumulative acetaminophen release from the 8 hour formulation is 42%, 57% and 89% at 2, 4 and 7 hours post-dissolution testing, respectively. The cumulative hydrocodone bitartrate release from the same formulation is 42%, 61% and 95% at the same time points. Therefore, this formulation exhibits a proportional release of acetaminophen and hydrocodone which are within 0%, 4% and 6% of each other. However, for some purposes, i.e., to achieve a particular desired in vitro release profile, or a particular plasma profile, a nonproportional release profile is contemplated.

Controlled release dosage forms exhibiting a stepwise increasing rate of release without the presence of surfactant are described in co-pending, commonly assigned patent application docket number ALZ5054, U.S. Ser. No. 60/497,162, filed Aug. 22, 2003. These dosage forms are characterized in part by two drug layer compositions that release consecutively to produce a stepwise or ascending rate of release from the dosage form. An “ascending” rate of release is defined as a first rate of release for a first period of time followed by a second rate of release for a second period of time, where the first rate of release is less than the second rate of release and each rate of release is substantially uniform over its period of time of delivery.

In contrast, it has been surprisingly discovered that oral osmotic dosage forms exhibiting an ascending drug release rate for an extended time period can be achieved using a single drug layer at a constant drug concentration, and a single osmotic push composition. No additional components such as an interior wall comprising pore formers or second drug layers are required to increase the drug release rate as the drug composition is delivered to the patient. It has also been surprisingly discovered that formulations prepared using a similar technology to that generally described in U.S. Pat. No. 6,368,626 provide an ascending release profile when adapted to deliver drug over a shorter period of time, that is when the dosage forms provide delivery of active agent in less than about 12 hours. This discovery is an advancement on the earlier development of high drug loading dosage forms that provide a uniform release rate of the active agent over a prolonged period of time.

The dosage forms are adapted to release active agent at an ascending release rate over a prolonged period of time, preferably 4 hours or more. Measurements of release rate are typically made in vitro, in acidified water to provide a simulation of conditions in gastric fluid, and are made over finite, incremental time periods to provide an approximation of instantaneous release rate. Information of such in vitro release rates with respect to a particular dosage form may be used to assist in selection of dosage form that will provide desired in vivo results. Such results may be determined by present methods, such as blood plasma assays and clinical observation, utilized by practitioners for prescribing available immediate release dosage forms.

It has been found that dosage forms having ascending release rate profiles can provide to a patient a substantially constant blood plasma concentration and a sustained therapeutic effect of active agent, after administration of the dosage form, over a prolonged period of time. The sustained release dosage forms can demonstrate less variability in drug plasma concentrations when administered over a 12 to 24-hour period than do immediate release formulations, which characteristically create significant peaks in drug concentration shortly or soon after administration to the subject. The ascending release rates can provide to a patient a zero order, ascending or descending plasma profile, depending on the rate of metabolism or excretion of the active agent, or depending on the patient's own medical condition (renal and hepatic sufficiency).

Sustained release dosage forms are provided comprising a pharmaceutically active agent and pharmaceutically acceptable salts thereof and adapted to release as an erodible solid over a prolonged period of time, wherein the dosage form provides an ascending rate of release of the pharmaceutically active agent for at least about 4 hours. In preferred embodiments, the sustained release dosage form provides an ascending rate of release of the pharmaceutically active agent for from about 5 to about 8 to 10 hours, or in some instances, for longer periods of time. The sustained release dosage forms are useful for delivering active agents even when the pharmaceutically active agent is required to be administered to a patient in a high dose, or where the active agent has a low solubility and/or poor dissolution rate.

Preferably, the rate of release exhibited by the dosage form at its maximum rate of release is at least 20% greater than its minimum rate of release, typically the rate of release observed during the first hour or two after administration or initiation of dissolution testing. Typically the maximum rate of release occurs when about 70% of the active agent is being released. In other embodiments, the maximum rate of release exhibited by the dosage form is at least 40% greater than the minimum release rate exhibited by the dosage form. In additional embodiments, the maximum rate of release exhibited by the dosage form is at least 60% greater than the minimum release rate exhibited by the dosage form.

In certain embodiments, the erodible solid further comprises a binding agent, and a disintegrant, and it can include a surfactant and an osmagent. Preferred binding agents include polyoxyalkylenes, hydroxyalkylcelluloses, hydroxyalkylalkylcelluloses, and polyvinylpyrrolidones, and the like. Preferred disintegrants include croscarmellose sodium, crospovidone, sodium alginate, sodium starch glycolate, and the like.

Preferably, the sustained release dosage form provides an ascending rate of release of the pharmaceutically active agent until about 70% of the active agent has been released, and after the ascending rate of release, there is a rapid decrease in release rate. Preferably, the dosage form releases at least 90% of the active agent within about 12 hours.

In particular embodiments, the erodible solid further comprises a surfactant, which can be either a nonionic or ionic surfactant. Nonionic surfactants preferably include poloxamers, polyoxyethylene esters, sugar ester surfactants, sorbitan fatty acid esters, glycerol fatty acid esters, polyoxyethylene ethers of high molecular weight aliphatic alcohols, polyoxyethylene 40 sorbitol lanolin derivatives, polyoxyethylene 75 sorbitol lanolin derivatives, polyoxyethylene 20 sorbitol lanolin derivatives, polyoxyethylene 50 sorbitol lanolin derivatives, polyoxyethylene 6 sorbitol beeswax derivatives, polyoxyethylene 20 sorbitol beeswax derivatives, polyoxyethylene derivatives of fatty acid esters of sorbitan, and mixtures thereof. Preferred nonionic surfactants include poloxamers, a fatty acid esters of polyoxyethylene, and sugar ester surfactants.

The sustained release dosage forms can provide an ascending release rate of any drug loading, such as a drug loading in the erodible solid of about 20 to about 95% by weight. In particular embodiments, the sustained release dosage forms are adapted to deliver high doses of active agent and to provide a high loading of the active agent. In certain embodiments, the pharmaceutically active agent is present in the erodible solid at a percent composition of at least 60 weight percent, and generally is present in the erodible solid in the range of from about 60 percent to about 95 percent by weight. In particular embodiments, the active agent is present in the erodible solid at a percent composition of from about 70 percent to about 90 percent by weight, or even at a drug loading of from about 75 percent to about 85 percent by weight. In certain embodiments, the erodible solid comprises from about 5 to about 15 percent by weight of a binding agent and a disintegrant. In additional embodiments, the erodible solid comprises from about 1 to about 15 percent by weight of a surfactant and can also contain an osmagent, typically less than 10-15 percent by weight.

In certain embodiments, the sustained release dosage form further comprises at least one additional pharmaceutically active agent in the erodible solid. The pharmaceutically active agents can be poorly soluble, can have similar or different solubilities, and are released from the dosage form at rates that can be proportional to each other. The pharmaceutically active agents typically have a solubility of less than about 50 mg/ml at 25° C., and may have a solubility of less than about 10 mg/ml at 25° C.

In certain additional embodiments, the sustained release dosage form can further comprise an immediate release drug coating comprising an effective dose of at least one pharmaceutically active agent, and where additional active agents are present in the sustained release dosage form, the immediate release drug coating can also comprise the additional active agents. The immediate release drug coating acts to provide an immediate dose of active agents to a patient, and the sustained release dosage form provides a sustained release of active agent over the entire dosing interval, thereby providing a therapeutically effective dose of the active agents to a patient in need thereof.

In additional embodiments, the sustained release dosage form comprises: (1) a semipermeable wall defining a cavity and including an exit orifice formed or formable therein; (2) a drug layer comprising a therapeutically effective amount of a pharmaceutically active agent and pharmaceutically acceptable salts thereof contained within the cavity and located adjacent to the exit orifice; (3) a push displacement layer contained within the cavity and located distal from the exit orifice; (4) a flow-promoting layer in between the inner surface of the semipermeable wall and at least the external surface of the drug layer that is opposite the wall; and provides an ascending rate of release of the pharmaceutically active agent for at least about 4 hours. The drug layer is exposed to the environment of use as an erodible composition. More preferably, the dosage form provides an ascending rate of release of the pharmaceutically active agent for from about 5 to about 8 hours, or for 10 hours or more. In preferred embodiments, after the ascending rate of release, the dosage forms exhibit a rapid decrease in release rate. Preferably, the dosage form releases at least 90% of the active agent within about 12 hours.

Preferably, the dosage form provides an ascending rate of release of the pharmaceutically active agent until about 70 percent of the active agent has been released. Typically the minimum release rate is exhibited by the dosage form when less than about 10-20% of the active agent has been released. In particular embodiments, the maximum rate of release exhibited by the dosage form is at least 20% greater than the minimum release rate. In additional embodiments, the maximum rate of release exhibited by the dosage form is at least 40% greater than the minimum release rate. In yet other embodiments, the maximum rate of release exhibited by the dosage form is at least 60% greater than the minimum release rate exhibited by the dosage form.

The sustained release dosage forms are particularly useful for delivering active agents even when the pharmaceutically active agent is required to be administered to a patient in a high dose, or where the active agent has a low solubility and/or poor dissolution rate.

In particular embodiments, the drug layer further comprises a binding agent, a disintegrant or mixtures thereof, and in certain other embodiments, the drug layer further comprises a surfactant and/or an osmagent. The surfactant can be a nonionic or ionic surfactant. Nonionic surfactants preferably include poloxamers, polyoxyethylene esters, sugar ester surfactants, sorbitan fatty acid esters, glycerol fatty acid esters, polyoxyethylene ethers of high molecular weight aliphatic alcohols, polyoxyethylene 40 sorbitol lanolin derivatives, polyoxyethylene 75 sorbitol lanolin derivatives, polyoxyethylene 20 sorbitol lanolin derivatives, polyoxyethylene 50 sorbitol lanolin derivatives, polyoxyethylene 6 sorbitol beeswax derivatives, polyoxyethylene 20 sorbitol beeswax derivatives, polyoxyethylene derivatives of fatty acid esters of sorbitan, and mixtures thereof. Preferred nonionic surfactants include poloxamers, a fatty acid esters of polyoxyethylene, and sugar ester surfactants.

In particular embodiments, the sustained release dosage forms are adapted to deliver high doses of active agent and to provide a high loading of the active agent. In certain embodiments, the pharmaceutically active agent is present in the drug layer at a percent composition of at least 60 weight percent, and generally is present in the drug layer in the range of from about 60 percent to about 95 percent by weight. In particular embodiments, the active agent is present in the drug layer at a percent composition of from about 70 percent to about 90 percent by weight, or even at a drug loading of from about 75 percent to about 85 percent by weight.

In certain embodiments, the sustained release dosage form further comprises at least one additional pharmaceutically active agent in the drug layer. The pharmaceutically active agents can have similar or different solubilities, and are released from the dosage form at rates that are proportional to the respective weights of each active agent in the dosage form. If non-proportional release rates are desired, the drug layer can be formed in multiple layers to vary the concentration of each active agent independently in each layer. Hence, an ascending release rate can result in an increasing release rate of one active agent and a decreasing release rate of an additional active agent even though the overall release rate is ascending.

In certain additional embodiments, the sustained release dosage form can further comprise an immediate release drug coating comprising an effective dose of at least one pharmaceutically active agent, and where additional active agents are present in the sustained release dosage form, the immediate release drug coating can also comprise the additional active agents. The immediate release drug coating acts to provide an immediate dose of active agents to a patient, and the sustained release dosage form provides a sustained release of active agent over the entire dosing interval, thereby providing a therapeutically effective dose of the active agents to a patient in need thereof.

The pharmaceutically active agent can be any solubility. Generally when the active agent is poorly soluble, the active agent has a solubility of less than about 50 mg/ml at 25° C., and may have a solubility of less than about 10 mg/ml at 25° C. The pharmaceutically active agent can be any pharmaceutically active agent, and in preferred embodiments is selected from a nonopioid analgesic agent, an antibiotic, an antiepileptic, or combinations thereof. In particular embodiments, at least one additional pharmaceutically active agent is included in the dosage form and can be selected from an opioid analgesic agent, a gastric protective agent, a 5-HT agonist, or other active agent.

The sustained release dosage forms can be used in methods for providing a sustained release of an increasing dose of a pharmaceutically active agent to a patient in need thereof. The sustained release dosage form is orally administered to a patient in need of treatment, and comprises a pharmaceutically active agent and pharmaceutically acceptable salts thereof adapted to release as an erodible solid over a prolonged period of time, and provides an ascending rate of release of the pharmaceutically active agent for at least about 4 hours.

In particular embodiments, methods for providing a sustained release of a therapeutically effective dose of a pharmaceutically active agent are provided, where the active agent is characterized by administration to a patient in a high dosage, low solubility and/or poor dissolution rate.

In additional embodiments, methods for providing a therapeutically effective dose of a pharmaceutically active agent to a patient in need thereof are provided, comprising orally administering a composition comprising a therapeutically effective amount of a pharmaceutically active agent present in a drug layer contained within a cavity defined by an at least partially semipermeable wall and having an exit means located adjacent thereto, a push displacement layer located within the cavity distal from the exit means providing a sustained release of the composition from the cavity when placed in an aqueous environment of use, and a flow-promoting layer located in between the inner surface of the semipermeable wall and at least the external surface of the drug layer that is opposite the wall, wherein the dosage form provides an ascending rate of release of the pharmaceutically active agent for at least about 4 hours. The method can further comprise utilizing a drug coating on the sustained release dosage form comprising a therapeutically effective amount of an immediate release therapeutic composition located on the outside surface of the at least partially semipermeable wall. The therapeutic composition preferably provides an ascending rate of release of the pharmaceutically active agent for from about 5 hours to about 8 hours or longer. In preferred embodiments, the drug layer comprises from about 60 to about 95% of the pharmaceutically active agent by weight, and more preferably from about 75 to about 85% of the pharmaceutically active agent by weight. In particular embodiments, the drug layer comprises from about 5 to about 15 percent by weight of a binding agent and a disintegrant, and optionally from about 1 to about 15 percent by weight of a surfactant.

In additional embodiments, methods for providing an effective concentration in the plasma of a patient of a pharmaceutically active agent that is metabolized relatively rapidly are provided, comprising orally administering a therapeutic composition comprising a pharmaceutically active agent and pharmaceutically acceptable salts thereof adapted to release as an erodible solid over a prolonged period of time, wherein the erodible solid comprises the pharmaceutically active agent, and wherein said therapeutic composition provides an ascending rate of release of the pharmaceutically active agent for at least about 4 hours. In preferred embodiments, the dosage form provides an ascending rate of release of the pharmaceutically active agent for from about 4 hours to about 8 hours.

The therapeutic composition can further comprise a drug coating (an “immediate release drug coating”) comprising a therapeutically effective amount of the pharmaceutically active agent sufficient to provide an immediate effect in a patient in need thereof. In particular embodiments, the therapeutic composition provides a substantially zero order plasma profile of the pharmaceutically active agent in the patient. In additional embodiments, the therapeutic composition provides an ascending plasma profile of the pharmaceutically active agent in the patient. In certain other embodiments, the therapeutic composition provides a declining plasma profile of the pharmaceutically active agent in the patient. In a preferred embodiment, the dosage form comprises an immediate release drug coating that provides a therapeutically effective amount of the pharmaceutically active agent in the plasma of the patient and the ascending rate of release provided by the therapeutic composition maintains the concentration of the pharmaceutically active agent in the therapeutic range in the plasma of the patient for a prolonged period of time.

In yet other embodiments, methods are provided for providing an effective dose of a pharmaceutically active agent to which tolerance develops relatively rapidly in a patient, comprising orally administering a therapeutic composition comprising an effective dose of a pharmaceutically active agent to which tolerance develops relatively rapidly contained in a drug layer, an osmotic push composition, an at least partially semipermeable wall, and an exit means in the wall for delivering the therapeutic composition from the dosage form, and a flow-promoting layer located in between the inner surface of the semipermeable wall and at least the external surface of the drug layer that is opposite the wall, wherein said drug layer and push composition are surrounded by the at least partially semipermeable wall, wherein the drug layer is exposed to the environment of use as an erodible composition, and further wherein said dosage form provides an ascending rate of release of the pharmaceutically active agent thereby providing increasing concentrations of the pharmaceutically active agent in the plasma of the patient.

In a preferred embodiment, a method for treating pain in a human patient in need thereof is provided, comprising orally administering a dosage form comprising a therapeutic composition comprising a nonopioid analgesic, an opioid analgesic and pharmaceutically acceptable salts thereof adapted to release as an erodible solid over a prolonged period of time, wherein said therapeutic composition provides an ascending rate of release of the nonopioid analgesic and the opioid analgesic for at least about 4 hours. In a preferred embodiment, the nonopioid analgesic and the opioid analgesic are released at rates that are proportional to each.

The embodiments of the dosage forms and methods of using them are described in greater detail below.

Drug Coating for Immediate Release of Active Agents

Drug coating formulations can optionally be included in the dosage forms described herein, and provide for the immediate release of active agents along with the sustained release of active agents provided by the sustained release component. Any drug coating formulations known in the art can be used in conjunction with the inventive dosage forms described herein, and can include any pharmaceutical agent, or combinations of agents, whether soluble or insoluble, and at any drug loading. Preferred drug coating formulations are described in co-pending commonly owned patent application Ser. No. 60/506,195, filed as Attorney Docket No. ARC 3363 P1 on Sep. 26, 2003, incorporated by reference herein in its entirety.

For certain preferred drug coatings, briefly, the drug coating can be formed from an aqueous coating formulation and includes at least one insoluble drug and a water soluble film-forming agent. Two or more insoluble drugs or one or more insoluble drugs in combination with one or more soluble drugs can be included in the drug coating. In a preferred embodiment, the drug coating includes an insoluble drug and a soluble drug. In a preferred embodiment, the insoluble drug included in the drug coating is a nonopioid analgesic, with acetaminophen being a particularly preferred insoluble drug. In an additional preferred embodiment, the soluble drug included in the drug coating is an opioid analgesic, with hydrocodone, oxycodone, hydromorphone, oxymorphone, codeine and methadone being particularly preferred soluble drugs.

In preferred embodiments, the drug coating includes from about 85 wt % to about 97 wt % insoluble drug, with coatings exhibiting an insoluble drug loading of about 90 wt % to about 93 wt % being particularly preferred. The total amount of soluble drug included in the drug coating preferably ranges from about 0.5 wt % to about 15 wt % soluble drug, and drug coatings including about 1 wt % to about 3 wt % soluble drug being most preferred. The total amount of insoluble drug included in a drug coating that incorporates both soluble and insoluble drugs preferably ranges from about 60 wt % to about 96.5 wt %, with drug coatings including about 75 wt % to about 89.5 wt % insoluble drug being more preferred, and drug coatings including about 89 wt % to about 90 wt % insoluble drug being most preferred. The total amount of drugs included in the drug coating ranges from about 85 wt % to about 97 wt %, and in preferred embodiments, the total amount of drug included in a drug coating ranges from about 90 wt % to about 93 wt %.

The film-forming agent included in the drug coating is water soluble and accounts for about 3 wt % to about 15 wt % of the drug coating, with drug coatings having about 7 wt % to about 10 wt % film-forming agent being preferred. The film-forming agent included in a drug coating is water soluble and preferably works to solubilize insoluble drug included in the drug coating. In addition, the film-forming agent included in a drug coating may be chosen such that the film-forming agent forms a solid solution with one or more insoluble drugs included in the drug coating. It is believed that drug loading and film forming characteristics of a drug coating are enhanced by selecting a film-forming agent that forms a solid solution with at least one of the one or more insoluble drugs included in the drug coating. A drug dissolved at the molecular level within the film-forming agent (a solid solution) is also expected to be more readily bioavailable because, as the drug coating breaks down or dissolves, the drug is released into the gastrointestinal tract and presented to the gastrointestinal mucosal tissue as discrete molecules.

In a preferred embodiment, the film-forming agent included in the drug coating is a film-forming polymer or a polymer blend including at least one film-forming polymer. Polymer materials used as the film-forming agent of a drug coating are water soluble. Examples of water soluble polymer materials that may be used as the film-forming polymer of a drug coating include, but are not limited to, hydroxypropylmethyl cellulose (“HPMC”), low molecular weight HPMC, hydroxypropyl cellulose (“HPC”) (e.g., Klucel®), hydroxyethyl cellulose (“HEC”) (e.g., Natrasol®), copovidone (e.g., Kollidon® VA 64), and PVA-PEG graft copolymer (e.g., Kollicoat® IR), and combinations thereof. A polymer blend or mixture may be used as the film forming agent in order to achieve a drug coating having characteristics that may not be achievable using a single film-forming polymer in combination with the drug or drugs to be included in the drug coating. For example, blends of HPMC and copovidone provide a film-forming agent that allows the formation of drug coatings that not only exhibit desirable drug loading characteristics, but also provide coatings that are aesthetically pleasing and exhibit desirable physical properties.

The drug coating can also include a viscosity enhancer. Because the drug coating is an aqueous coating that includes an insoluble drug, the drug coating is typically coated from an aqueous suspension formulation. In order to provide a drug coating with substantially uniform drug distribution from a suspension formulation, however, the suspension formulation should provide a substantially uniform dispersion of the insoluble drug included in the coating. Depending on the relative amounts and nature of the film-forming agent and the drugs included in a drug coating, a viscosity enhancer can be included in a drug coating to facilitate the creation of a coating formulation that exhibits sufficient viscosity to provide a substantially uniform drug dispersion and facilitates the production of a drug coating having a substantially uniform distribution of insoluble drug. A viscosity enhancer included in a drug coating is preferably water-soluble and can be a film-forming agent. Examples of viscosity enhancers that may be used in a drug coating include, but are not limited to, HPC (e.g., Klucel®), HEC (e.g., Natrasol®), Polyox® water soluble resin products, and combinations thereof.

The precise amount of viscosity enhancing material included in the drug coating will vary, depending on the amounts and type of film-forming polymer and drug materials to be used in the drug coating. However, where included in a drug coating, a viscosity enhancer will typically account for 5 wt %, or less, of the drug coating. Preferably, a drug coating includes 2 wt %, or less, viscosity enhancer, and in particularly preferred embodiments, the drug coating includes 1 wt %, or less, viscosity enhancer.

The drug coating can also include a disintegrating agent that increases the rate at which the drug coating disintegrates after administration. Because the drug coating typically includes a large amount of insoluble drug, the drug coating may not break down or disintegrate as rapidly as desired after administration. A disintegrating agent included in a coating is a water swellable material that works to structurally compromise the coating as the disintegrating agent absorbs water and swells. Disintegrating agents that may be used in the drug coating include, but are not limited to modified starches, modified cellulose, and cross-linked polyvinylpyrrolidone materials. Specific examples of disintegrating agents that may be used in the drug coating and are commercially available include Ac-Di-Sol®, Avicel®, and PVP XL-10. Where included in the drug coating, a disintegrating agent typically accounts for up to about 6 wt % of the coating, with coatings incorporating from about 0.5 wt % to about 3 wt % being preferred and coatings incorporating from about 1 wt % to about 3 wt % being particularly preferred.

The drug coating can also include a surfactant to increase the rate at which the drug coating dissolves or erodes after administration. The surfactant serves as a “wetting” agent that allows aqueous liquids to more easily spread across or penetrate the drug coating. Surfactants suitable for use in a drug coating are preferably solid at 25° C. Examples of surfactants that may be used in the drug coating include, but are not limited to, surface active polymers, such as Poloxamer and Pluronic® surfactants. Where a surfactant is included in a drug coating, the surfactant will typically account for up to about 6 wt % of the drug coating, with drug coatings including about 0.5 wt % to about 3 wt % surfactant being preferred, and drug coatings including about 1 wt % to about 3 wt % surfactant being particularly preferred.

In one embodiment of the drug coating, the film-forming agent includes a polymer blend formed of copovidone and HPMC. Where such a polymer blend is used as the film-forming agent of the drug coating, the amounts of copovidone and HPMC can vary, as desired, to achieve a drug coating having desired physical and drug-loading characteristics. However, where the film-agent included in a drug coating is formed of a blend of copovidone and HPMC, the copovidone and HPMC are preferably included at a wt/wt ratio about 0.6:1 to about 0.7:1 copovidone to HPMC, with a wt/wt ratio of 1:1.5 being most preferred. Blends of HPMC and copovidone provide drug coatings that are aesthetically pleasing and are believed to be sufficiently robust to withstand further processing and an extended shelf life. Moreover, it is believed that copovidone can work to solubilize insoluble drug included in a drug coating, providing a drug coating that includes a solid solution of insoluble drug.

In a preferred embodiment, the drug coating includes a blend of HPMC and copovidone as the film-forming agent and a nonopioid analgesic as an insoluble drug, preferably acetaminophen.

In yet another embodiment, the drug coating includes a blend of HPMC and copovidone as the film-forming agent, an insoluble nonopioid analgesic, and a soluble opioid analgesic. In a specific example of such an embodiment, the drug coating includes an opioid analgesic, such as hydrocodone and pharmaceutically acceptable salts thereof. A dosage form that includes the combination of acetaminophen or ibuprofen and an opioid analgesic provides a combination of analgesic, anti-inflammatory, anti-pyretic, and antitussive actions.

In even further embodiments, the drug coating includes a blend of HPMC and copovidone as the film-forming agent, an insoluble nonopioid analgesic, a soluble opioid analgesic, and a viscosity enhancing agent or a disintegrating agent. In a specific example of such an embodiment, the drug coating includes between about 1 wt % and about 2 wt % of a viscosity enhancing agent, such as HPC. In another example of such an embodiment, the drug coating includes between about 0.5 wt % and about 3 wt % disintegrating agent, and in yet another example of such an embodiment, the drug coating includes between about 0.5 wt % and about 3 wt % of a surfactant.

The drug coating is not only capable of achieving high drug loading, but where the drug coating includes two or more different drugs, it has been found that the drug coating releases the different drugs in amounts that are directly proportional to the amounts of the drugs included in the drug coating. The proportional release is observed even where drugs exhibiting drastically different solubility characteristics, such as acetaminophen and hydrocodone, are included in the drug coating. In addition a drug coating according to the present invention releases substantially all of the drug included therein. Such performance characteristics facilitate reliable and predictable drug delivery performance, and allow formulation of drug coatings that deliver two or more drugs at a wide range of different ratios.

In another aspect, a coating formulation can be used to provide a drug coating. The coating suspension includes the materials used to form a drug coating which is dissolved or suspended, depending on the material, within one or more solvents or solutions. The one or more solvents included in a coating suspension are not organic solvents, and are preferably aqueous solvents. Aqueous solvents that may be used in a coating suspension include, but are not limited to, purified water, pH adjusted water, acidified water, or aqueous buffer solutions. In a preferred embodiment, the aqueous solvent included in a coating suspension is purified water USP. The coating formulation is preferably an aqueous formulation and avoids the potential problems and disadvantages that can result from the use of organic solvents in formulating coating compositions.

As the drug coating includes at least one insoluble drug, the coating formulation is typically prepared as an aqueous suspension using any suitable process, and in preferred embodiments the coating formulation is formulated to facilitate production of drug coatings through a known coating process, such as, for example, pan coating, fluid bed coating, or any other standard coating processes suitable for providing a drug coating. Though the precise amount of solvent used in a coating suspension may vary depending on, for example, the materials to be included in the finished drug coating, the desired coating performance of the coating suspension and the desired physical characteristics of the finished drug coating, a coating suspension typically includes up to about 30 wt % solids content, with the remainder of the coating suspension consisting of the desired solvent. A preferred embodiment of a coating suspension includes about 80 wt % of a desired aqueous solvent and about 20 wt % solids content. The coating suspension is formulated to exhibit a viscosity that is low enough to facilitate spray coating of drug coating, yet is high enough to maintain a substantially uniform dispersion of the insoluble drug included in the coating suspension during a coating process.

In preparing a coating formulation, the drug loaded into the coating formulation can be provided in micronized form. By reducing the particle size of the drug loaded into a coating formulation, a more cosmetically smooth drug coating may be achieved. In addition, by reducing the particle size of the drug material loaded into a coating formulation, the dissolution rate of the drug when released from the drug coating prepared by the coating formulation may be improved, particularly where the drug is an insoluble drug. In one embodiment of the coating formulation, the coating formulation includes a micronized drug material exhibiting an average particle size of less than 100 microns. In another embodiment, the coating formulation includes a micronized drug material exhibiting an average particle size of less than 50 microns, and in yet another embodiment, the coating formulation includes a micronized drug material exhibiting an average particle size of less than 10 microns. Micronization of the drug material can be readily achieved through processes well known in the art, such as, for example, known bead milling, jet milling or microprecipitation processes, and particle size can be measured using any conventional particle size measuring technique, such as sedimentation field flow fractionation, photon correlation spectroscopy or disk centrifugation.

The solids dissolved or suspended in a coating formulation are loaded into the coating formulation in the same relative amounts as are used in a drug coating. For example, the drug included in a coating formulation accounts for about 85 wt % to about 97 wt % of the solids loaded into the coating formulation. In preferred embodiments, the drug included in a coating formulation accounts for about 90 wt % to about 93 wt % of the solids loaded into the coating formulation. The film-forming agent included in a coating formulation accounts for about 3 wt % to about 15 wt % of the solids loaded into the coating formulation, and in preferred embodiments, the film-forming agent included in a coating formulation accounts for about 7 wt % to about 10 wt % of the solids loaded into the coating formulation. Where included, a viscosity enhancer will typically account for 5 wt %, or less, of the solids included in a coating formulation. Coating formulations wherein the viscosity enhancer accounts for 2 wt %, or less, of the solids are preferred, and in particularly preferred embodiments, a viscosity enhancer included in a coating formulation accounts for 1 wt %, or less, of the solids included in the coating formulation. If the coating to be formed by the coating formulation is to include a disintegrating agent, the disintegrating agent typically accounts for up to about 6 wt % of the solids included in the coating formulation. In preferred embodiments, a disintegrating agent will account for about 0.5 wt % to about 3 wt % of the solids included in the coating formulation, and in particularly preferred embodiments of a coating formulation including a disintegrating agent, the disintegrating agent accounts for about 1 wt % to about 3 wt % of the solids included in the coating formulation. Where a surfactant is included in a drug coating according to the present invention, the surfactant will typically account for up to about 6 wt % of the solids included in the coating formulation. Preferably, if a surfactant is included in a coating formulation, the surfactant will account for about 0.5 wt % to about 3 wt % of the solids included in the coating formulation, and in particularly preferred embodiments of a coating formulation that includes a surfactant, the surfactant accounts for about 1 wt % to about 3 wt % of the solids included in the coating formulation. Preparation of osmotic dosage forms containing active agents

The OROS® technology provides tunable sustained release dosage forms that can provide sustained release of one or more active agents, with or without the use of a drug coating providing immediate release of drug. Various types of osmotic dispensers include elementary osmotic pumps, such as those described in U.S. Pat. No. 3,845,770, mini-osmotic pumps such as those described in U.S. Pat. Nos. 3,995,631, 4,034,756 and 4,111,202, and multi-chamber osmotic systems referred to as push-pull, push-melt and push-stick osmotic pumps, such as those described in U.S. Pat. Nos. 4,320,759, 4,327,725, 4,449,983, 4,765,989 and 4,940,465, 6,368,626 to Bhatt, all of which are incorporated herein by reference. Specific adaptations of OROS® that can be used preferably include the OROS® Push-Stick™ System. A significant advantage to osmotic systems is that operation is substantially pH-independent and thus continues at the osmotically determined rate throughout an extended time period even as the dosage form transits the gastrointestinal tract and encounters differing microenvironments having significantly different pH values. Sustained release can be provided for times as short as a few hours or for as long as the dosage form resides in the gastrointestinal tract.

Osmotic dosage forms utilize osmotic pressure to generate a driving force for imbibing fluid into a compartment formed, at least in part, by a semi-permeable wall that permits diffusion of water but not drug or osmagents, if present. In these osmotic dosage forms, the active agent reservoir(s) is typically formed with an active agent compartment, containing a pharmaceutical agent in the form of a solid, liquid or suspension, as the case may be, and an expandable “push” compartment of a hydrophilic polymer that will imbibe fluid from the stomach, swell and force the active agent out of the dosage form and into the environment of use.

A review of such osmotic dosage forms is found in Santus and Baker (1995), “Osmotic drug delivery: a review of the patent literature,” Journal of Controlled Release 35: 1-21, incorporated in its entirety by reference herein. In particular, the following U.S. Patents, owned by the assignee of the present application, ALZA Corporation, and directed to osmotic dosage forms, are each incorporated in their entirety herein: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; 5,156,850; 5,912,268; 6,375,978; 6,368,626; 6,342,249; 6,333,050; 6,287,295; 6,283,953; 6,270,787; 6,245,357; and 6,132,420.

The core of the dosage form typically comprises a drug layer comprising a dry composition or substantially dry composition formed by compression of the binding agent and the analgesic agents as one layer and the expandable or push layer as the second layer. By “dry composition” or “substantially dry composition” is meant that the composition forming the drug layer of the dosage form is expelled from the dosage form in a plug-like state, the composition being sufficiently dry or so highly viscous that it does not readily flow as a liquid stream from the dosage form under the pressure exerted by the push layer. The drug layer itself has very little osmotic activity relative to the push layer, as the drug, binding agent and disintegrant are not well hydrated, and the drug layer does not flow out of the dosage form as a slurry or suspension. The drug layer is exposed to the environment of use as an erodible composition, in contrast to alternative osmotic dosage forms in which the drug layer is exposed to the environment of use as a slurry or suspension. The drug layer is an erodible composition because it includes very little if any osmagent due to the high drug loading provided as well as the poor solubility of the drug to be delivered.

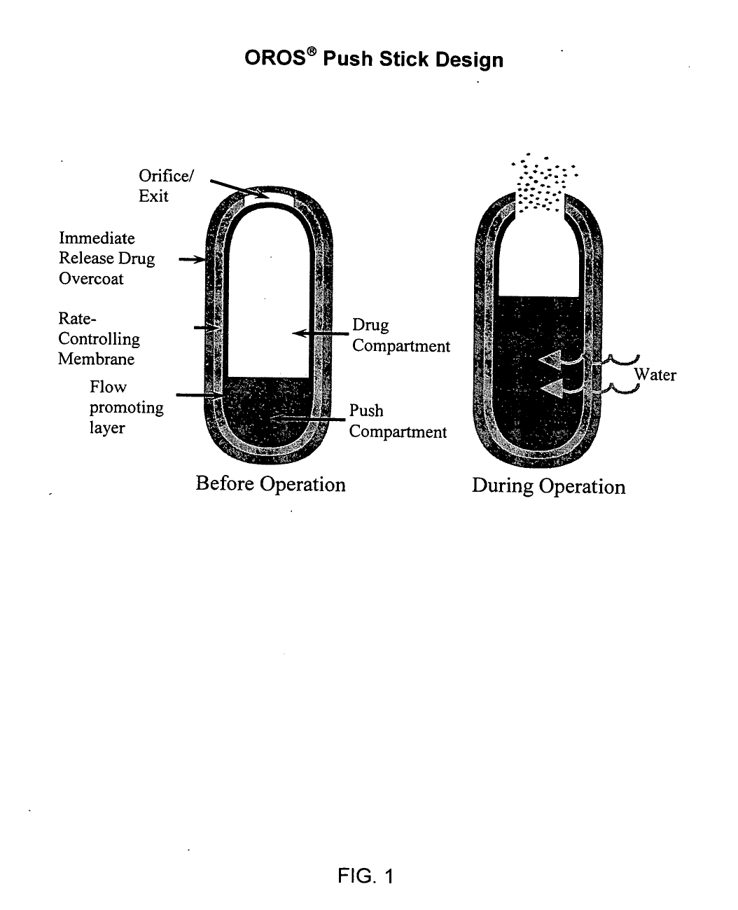

Compression techniques are known in the art and exemplified in Example 1. The expandable layer pushes the drug layer from the exit orifice as the push layer imbibes fluid from the environment of use, and the exposed drug layer will be eroded to release the drug into the environment of use. This may be seen with reference to FIG. 1. Upon release from the dosage form, the drug layer imbibes water causing the disintegrant to swell and soluble agents to dissolve, allowing the erodible solid to disperse and the analgesic agents to dissolve in the fluid at the environment of use. This “push-stick” formulation is a preferred dosage form and is described in greater detail below.

A particular embodiment of the osmotic dosage form comprises: a semipermeable wall defining a cavity and including an exit orifice formed or formable therein, a drug layer comprising at least one pharmaceutically active agent contained within the cavity and located adjacent to the exit orifice, a push displacement layer contained within the cavity and located distal from the exit orifice, and a flow-promoting layer between the inner surface of the semipermeable wall and at least the external surface of the drug layer that is opposite the wall. The dosage form provides an in vitro rate of release of the active agents for up to about 12 hours after being contacted with water in the environment of use.

Composition of the Osmotic Dosage Forms

A preferred embodiment of a dosage form of this invention having the “push-stick” configuration is illustrated in FIG. 1 prior to its administration to a subject, during operation and after delivery of the active agent. The dosage form comprises a wall defining a cavity and an exit orifice. Within the cavity and remote from the exit orifice is a push displacement layer, and a drug layer is located within cavity adjacent the exit orifice. A flow-promoting layer extends at least between the drug layer and the inner surface of the wall, and can extend between the inner surface of the wall and the push displacement layer.

The dosage form can be at any drug loading, and preferably is at a loading of active agent of at least about 20% by weight. In particular embodiments, the dosage form is at high drug loading, i.e., 60% or greater, but more generally 70% or greater, active agent in the drug layer based on the overall weight of the drug layer, and is exposed to the environment of use as an erodible composition. The drug layer comprises a composition formed of at least one active agent in combination with a disintegrant, a binding agent, and optionally a surfactant, and an osmagent, or mixtures thereof. The active agent can be an insoluble drug such as a nonopioid analgesic.

The binding agent is generally a hydrophilic polymer that contributes to the release rate of active agent and controlled delivery pattern, such as a hydroxyalkylcellulose, a hydroxypropylalkylcellulose, a poly(alkylene) oxide, or a polyvinylpyrrolidone, or mixtures thereof. Representative examples of these hydrophilic polymers are poly(alkylene oxides) of 100,000 to 750,000 number-average molecular weight, including without limitation poly(ethylene oxide), poly(methylene oxide), poly(butylene oxide) and poly(hexylene oxide); poly(carboxymethylcelluloses) of 40,000 to 400,000 number-average molecular weight, represented by poly(alkali carboxymethylcellulose), such as poly(sodium carboxymethylcellulose), poly(potassium carboxymethylcellulose) and poly(lithium carboxymethylcellulose); hydroxyalkylcelluloses of 9,200 to 125,000 number-average molecular weight such as hydroxypropylcellulose, hydroxypropylalkylcelluloses such as hydroxypropylalkylcellulose of 9,200 to 125,000 number-average molecular weight, including without limitation, hydroxypropylethylcellulose, hydroxypropyl methylcellulose, hydroxypropylbutylcellulose and hydroxypropylpentylcellulose; and poly(vinylpyrrolidones) of 7,000 to 75,000 number-average molecular weight. Preferred among those polymers are the poly(ethylene oxide) of 100,000-300,000 number average molecular weight and hydroxyalkylcelluloses. Carriers that erode in the gastric environment, i.e., bioerodible carriers, are especially preferred.

Surfactants and disintegrants may be utilized in the carrier as well. Disintegrants generally include starches, clays, celluloses, algins and gums and crosslinked starches, celluloses and polymers. Representative disintegrants include corn starch, potato starch, croscarmellose, crospovidone, sodium starch glycolate, Veegum HV, methylcellulose, agar, bentonite, carboxymethylcellulose, low substituted carboxymethylcellulose, alginic acid, guar gum and the like. A preferred disintegrant is croscarmellose sodium.

Exemplary surfactants are those having an HLB value of between about 10-25, such as polyethylene glycol 400 monostearate, polyoxyethylene-4-sorbitan monolaurate, polyoxyethylene-20-sorbitan monooleate, polyoxyethylene-20-sorbitan monopalmitate, polyoxyethylene-20-monolaurate, polyoxyethylene-40-stearate, sodium oleate and the like. Surfactants that are useful generally include ionic surfactants, including anionic, cationic, and zwitterionic surfactants, and nonionic surfactants. Nonionic surfactants are preferred in certain embodiments and include, for example, fatty acid esters of polyoxyethylene such as polyoxyethylene steroidal esters and polyoxyl stearates, including but not limited to polyoxyl 40 stearate, polyoxyl 50 stearate, polyoxyl 100 stearate, polyoxyl 12 distearate, polyoxyl 32 distearate, and polyoxyl 150 distearate, and other Myrj™ series of surfactants, or mixtures thereof. Yet another class of surfactant that is useful in the drug layer are the triblock co-polymers of ethylene oxide/propylene oxide/ethylene oxide, also known as poloxamers, having the general formula HO(C₂H₄O)ₐ(—C₃H₆O)_{b}(C₂H₄O)ₐH, available under the tradenames Pluronic and Poloxamer. In this class of surfactants, the hydrophilic ethylene oxide ends of the surfactant molecule and the hydrophobic midblock of propylene oxide of the surfactant molecule serve to dissolve and suspend the drug. These surfactants are solid at room temperature. Other useful surfactants include sugar ester surfactants, sorbitan fatty acid esters such as sorbitan monolaurate, sorbitan monopalmitate, sorbitan monostearate, sorbitan tristearate, and other Span™ series surfactants, glycerol fatty acid esters such as glycerol monostearate, polyoxyethylene derivatives such as polyoxyethylene ethers of high molecular weight aliphatic alcohols (e.g., Brij 30, 35, 58, 78 and 99), polyoxyethylene stearate (self emulsifying), polyoxyethylene 40 sorbitol lanolin derivative, polyoxyethylene 75 sorbitol lanolin derivative, polyoxyethylene 6 sorbitol beeswax derivative, polyoxyethylene 20 sorbitol beeswax derivative, polyoxyethylene 20 sorbitol lanolin derivative, polyoxyethylene 50 sorbitol lanolin derivative, polyoxyethylene 23 lauryl ether, polyoxyethylene 2 cetyl ether with butylated hydroxyanisole, polyoxyethylene 10 cetyl ether, polyoxyethylene 20 cetyl ether, polyoxyethylene 2 stearyl ether, polyoxyethylene 10 stearyl ether, polyoxyethylene 20 stearyl ether, polyoxyethylene 21 stearyl ether, polyoxyethylene 20 oleyl ether, polyoxyethylene derivatives of fatty acid esters of sorbitan such as polyoxyethylene 4 sorbitan monostearate, polyoxyethylene 20 sorbitan tristearate, and other Tween™ series of surfactants, phospholipids and phospholipid fatty acid derivatives such as lecithins, fatty amine oxides, fatty acid alkanolamides, propylene glycol monoesters and monoglycerides, such as hydrogenated palm oil monoglyceride, hydrogenated soybean oil monoglyceride, hydrogenated palm stearine monoglyceride, hydrogenated vegetable monoglyceride, hydrogenated cottonseed oil monoglyceride, refined palm oil monoglyceride, partially hydrogenated soybean oil monoglyceride, cotton seed oil monoglyceride sunflower oil monoglyceride, sunflower oil monoglyceride, canola oil monoglyceride, succinylated monoglycerides, acetylated monoglyceride, acetylated hydrogenated vegetable oil monoglyceride, acetylated hydrogenated coconut oil monoglyceride, acetylated hydrogenated soybean oil monoglyceride, glycerol monostearate, monoglycerides with hydrogenated soybean oil, monoglycerides with hydrogenated palm oil, succinylated monoglycerides and monoglycerides, monoglycerides and rapeseed oil, monoglycerides and cottonseed oils, monoglycerides with propylene glycol monoester sodium stearoyl lactylate silicon dioxide, diglycerides, triglycerides, Triton-X series of surfactants produced from octylphenol polymerized with ethylene oxide, where the number “100” in the trade name is indirectly related to the number of ethylene oxide units in the structure, (e.g., Triton X-100™ has an average of N=9.5 ethylene oxide units per molecule, with an average molecular weight of 625) and having lower and higher mole adducts present in lesser amounts in commercial products, as well as compounds having a similar structure to Triton X-100™, including Igepal CA-630™ and Nonidet P-40M (NP-40™, N-lauroylsarcosine, Sigma Chemical Co., St. Louis, Mo.), and the like. Any of the above surfactants can also include optional added preservatives such as butylated hydroxyanisole and citric acid. In addition, any hydrocarbon chains in the surfactant molecules can be saturated or unsaturated, hydrogenated or unhydrogenated.

An especially preferred family of surfactants are the poloxamer surfactants, which are a:b:a triblock co-polymers of ethylene oxide:propylene oxide:ethylene oxide. The “a” and “b” represent the average number of monomer units for each block of the polymer chain. These surfactants are commercially available from BASF Corporation of Mount Olive, N.J., in a variety of different molecular weights and with different values of “a” and “b” blocks. For example, Lutrol® F127 has a molecular weight range of 9,840 to 14,600 and where “a” is approximately 101 and “b” is approximately 56, Lutrol® F87 represents a molecular weight of 6,840 to 8,830 where “a” is 64 and “b” is 37, Lutrol® F108 represents an average molecular weight of 12,700 to 17,400 where “a” is 141 and “b” is 44, and Lutrol® F68 represents an average molecular weight of 7,680 to 9,510 where “a” has a value of about 80 and “b” has a value of about 27.

Other surfactants are the sugar ester surfactants, which are sugar esters of fatty acids. Such sugar ester surfactants include sugar fatty acid monoesters, sugar fatty acid diesters, triesters, tetraesters, or mixtures thereof, although mono- and di-esters are most preferred. Preferably, the sugar fatty acid monoester comprises a fatty acid 5 having from 6 to 24 carbon atoms, which may be linear or branched, or saturated or unsaturated C₆ to C₂₄ fatty acids. The C₆ to C₂₄ fatty acids include C₆, C₇, C₉, C₉, C₁₀, C₁₁, C₁₂, C₁₃, C₁₄, C₁₅, C₁₆, C₁₇, C₁₈, C₁₉, C₂₀, C₂₁, C₂₂, C₂₃, and C₂₄ in any subrange or combination. These esters are preferably chosen from stearates, behenates, cocoates, arachidonates, palmitates, myristates, laurates, carprates, oleates, laurates and their mixtures.

Preferably, the sugar fatty acid monoester comprises at least one saccharide unit, such as sucrose, maltose, glucose, fructose, mannose, galactose, arabinose, xylose, lactose, sorbitol, trehalose or methylglucose. Disaccharide esters such as sucrose esters are most preferable, and include sucrose cocoate, sucrose monooctanoate, sucrose monodecanoate, sucrose mono- or dilaurate, sucrose monomyristate, sucrose mono- or dipalmitate, sucrose mono- and distearate, sucrose mono-, di- or trioleate, sucrose mono- or dilinoleate, sucrose polyesters, such as sucrose pentaoleate, hexaoleate, heptaoleate or octooleate, and mixed esters, such as sucrose palmitate/stearate.

Particularly preferred examples of these sugar ester surfactants include those sold by the company Croda Inc of Parsippany, N.J. under the names Crodesta F10, F50, F160, and F110 denoting various mono-, di- and mono/di ester mixtures comprising sucrose stearates, manufactured using a method that controls the degree of esterification, such as described in U.S. Pat. No. 3,480,616. These preferred sugar ester surfactants provide the added benefit of tableting ease and nonsmearing granulation.

Use may also be made of those sold by the company Mitsubishi under the name Ryoto Sugar esters, for example under the reference B370 corresponding to sucrose behenate formed of 20% monoester and 80% di-, tri- and polyester. Use may also be made of the sucrose mono- and dipalmitate/stearate sold by the company Goldschmidt under the name “Tegosoft PSE”. Use may also be made of a mixture of these various products. The sugar ester can also be present in admixture with another compound not derived from sugar; and a preferred example includes the mixture of sorbitan stearate and of sucrose cocoate sold under the name “Arlatone 2121” by the company ICI. Other sugar esters include, for example, glucose trioleate, galactose di-, tri-, tetra- or pentaoleate, arabinose di-, tri- or tetralinoleate or xylose di-, tri- or tetralinoleate, or mixtures thereof. Other sugar esters of fatty acids include esters of methylglucose include the distearate of methylglucose and of polyglycerol-3 sold by the company Goldschmidt under the name of Tegocare 450. Glucose or maltose monoesters can also be included, such as methyl O-hexadecanoyl-6-D-glucoside and O-hexadecanoyl-6-D-maltose. Certain other sugar ester surfactants include oxyethylenated esters of fatty acid and of sugar include oxyethylenated derivatives such as PEG-20 methylglucose sesquistearate, sold under the name “Glucamate SSE20”, by the company Amerchol.

A resource of surfactants including solid surfactants and their properties is available in McCutcheon's Detergents and Emulsifiers, International Edition 1979 and McCutcheon's Detergents and Emulsifiers, North American Edition 1979. Other sources of information on properties of solid surfactants include BASF Technical Bulletin Pluronic & Tetronic Surfactants 1999 and General Characteristics of Surfactants from ICI Americas Bulletin 0-1 10/80 5M, and Eastman Food Emulsifiers Bulletin ZM-1K October 1993.

One of the characteristics of surfactants tabulated in these references is the HLB value, or hydrophilic lipophilic balance value. This value represents the relative hydrophilicity and relative hydrophobicity of a surfactant molecule. Generally, the higher the HLB value, the greater the hydrophilicity of the surfactant while the lower the HLB value, the greater the hydrophobicity. For the Lutrol® molecules, for example, the ethylene oxide fraction represents the hydrophilic moiety and the propylene oxide fraction represents the hydrophobic fraction. The HLB values of Lutrol® F127, F87, F108, and F68 are respectively 22.0, 24.0, 27.0, and 29.0. The preferred sugar ester surfactants provide HLB values in the range of about 3 to about 15. The most preferred sugar ester surfactant, Crodesta F160 is characterized by having a HLB value of 14.5.

Ionic surfactants include cholic acids and derivatives of cholic acid such as deoxycholic acid, ursodeoxycholic acid, taurocholic acid, taurodeoxycholic acid, taurochenodeoxycholic acid, and salts thereof, and anionic surfactants, the most common example of which is sodium dodecyl (or lauryl) sulfate. Zwitterionic or amphoteric surfactants generally include a carboxylate or phosphate group as the anion and an amino or quaternary ammonium moiety as the cation. These include, for example, various polypeptides, proteins, alkyl betaines, and natural phospholipids such as lecithins and cephalins, alkyl-beta-aminopropionates and 2-alkyl-imidazoline quaternary ammonium salts, as well as the CHAPS series of surfactants (e.g., 3-[3-Cholamidopropyl) dimethylammoniol]-1-propanesulfonate hydrate available from Aldrich), and the like.

Surfactants typically have poor cohesive properties and therefore do not compress as hard, durable tablets. Furthermore, surfactants are in the physical form of liquid, pastes, or waxy solids at standard temperatures and conditions and are inappropriate for tableted oral pharmaceutical dosage forms. The aforementioned surfactants have been found to function by enhancing the solubility and potential bioavailability of low solubility drugs delivered in high doses.

Surfactant can be included as one surfactant or as a blend of surfactants. The surfactants are selected such that they have values that promote the dissolution and solubility of the drug. A high HLB surfactant can be blended with a surfactant of low HLB to achieve a net HLB value that is between them, if a particular drug requires the intermediate HLB value. The surfactant is selected depending upon the drug being delivered; such that the appropriate HLB grade is utilized.

The pharmaceutically active agent can be provided in the drug layer in amounts of from about 1 microgram to about 1000 mg per dosage form, and more typically from about 10 to about 600 mg, depending upon the required dosing level that must be maintained over the delivery period, i.e., the time between consecutive administrations of the dosage forms. In an exemplary embodiment, the pharmaceutically active agent is acetaminophen (e.g., 500 mg). Generally, loading of active agent in the dosage forms will provide doses to a subject ranging up to about 3000 mg per day, more usually up to about 1000 to 2000 mg per day, depending on the level of medication required by the patient. Occasionally very high doses of up to about 10,000 mg per day are required.

An additional pharmaceutically active agent can be provided in the drug layer in amounts of from 1 microgram to 500 mg per dosage form, and more typically from about 10 mg to about 100 mg, depending upon the required dosing level that must be maintained over the delivery period, i.e., the time between consecutive administrations of the dosage forms. In an exemplary preferred embodiment, the additional active agent is an opioid analgesic (e.g., hydrocodone or hydromorphone) and is included in a smaller amount (e.g., 15 mg). Generally, loading of an additional pharmaceutically active agent in the dosage forms will provide doses of the active agents to a subject ranging up to about 2000 mg per day, more typically between about 10 to 60 or 600 mg per day, depending on the level of medication required by the patient.

The push layer is an expandable layer having a push-displacement composition in direct or indirect contacting layered arrangement with the drug layer. The push layer generally comprises a polymer that imbibes an aqueous or biological fluid and swells to push the drug composition through the exit means of the device. Representatives of fluid-imbibing displacement polymers comprise members selected from poly(alkylene oxide) of 1 million to 15 million number-average molecular weight, as represented by poly(ethylene oxide) and poly(alkali carboxymethylcellulose) of 500,000 to 3,500,000 number-average molecular weight, wherein the alkali is sodium, potassium or lithium. Examples of additional polymers for the formulation of the push-displacement composition comprise osmopolymers comprising polymers that form hydrogels, such as Carbopol® acidic carboxypolymer, a polymer of acrylic cross-linked with a polyallyl sucrose, also known as carboxypolymethylene, and carboxyvinyl polymer having a molecular weight of 250,000 to 4,000,000; Cyanamer® polyacrylamides; cross-linked water swellable indenemaleic anhydride polymers; Good-rites polyacrylic acid having a molecular weight of 80,000 to 200,000; Aqua-Keeps® acrylate polymer polysaccharides composed of condensed glucose units, such as diester cross-linked polygluran; and the like. Representative polymers that form hydrogels are known to the prior art in U.S. Pat. No. 3,865,108, issued to Hartop; U.S. Pat. No. 4,002,173, issued to Manning; U.S. Pat. No. 4,207,893, issued to Michaels; and in Handbook of Common Polymers, Scott and Roff, Chemical Rubber Co., Cleveland, Ohio.

The osmagent, also known as osmotic solute and osmotically effective agent, which exhibits an osmotic pressure gradient across the outer wall and subcoat, comprises a member selected from the group consisting of sodium chloride, potassium chloride, lithium chloride, magnesium sulfate, magnesium chloride, potassium sulfate, sodium sulfate, lithium sulfate, potassium acid phosphate, mannitol, urea, inositol, magnesium succinate, tartaric acid raffinose, sucrose, glucose, lactose, sorbitol, inorganic salts, organic salts and carbohydrates.

A flow promoting layer (also called the subcoat for brevity) is in contacting relationship with the inner surface of the semipermeable wall and at least the external surface of the drug layer that is opposite wall; although the flow-promoting layer may, and preferably will, extend to, surround and contact the external surface of the push displacement layer. The wall typically will surround at least that portion of the external surface of the drug layer that is opposite the internal surface of the wall. The flow-promoting layer may be formed as a coating applied over the compressed core comprising the drug layer and the push layer. The outer semipermeable wall surrounds and encases the inner flow-promoting layer. The flow-promoting layer is preferably formed as a subcoat of at least the surface of the drug layer, and optionally the entire external surface of the compacted drug layer and the push displacement layer. When the semipermeable wall is formed as a coat of the composite formed from the drug layer, the push layer and the flow-promoting layer, contact of the semipermeable wall with the flow-promoting layer is assured.

The flow-promoting layer facilitates release of drug from the dosage forms of the invention by reducing the frictional forces between the semipermeable wall 2 and the outer surface of the drug layer, thus allowing for more complete delivery of drug from the device. Particularly in the case of active compounds having a high cost, such an improvement presents substantial economic advantages since it is not necessary to load the drug layer with an excess of drug to insure that the minimal amount of drug required will be delivered.

The flow-promoting layer typically may be 0.01 to 5 mm thick, more typically 0.5 to 5 mm thick, and it comprises a member selected from hydrogels, gelatin, low molecular weight polyethylene oxides (e.g., less than 100,000 MW), hydroxyalkylcelluloses (e.g., hydroxyethylcellulose), hydroxypropylcelluloses, hydroxyisopropylcelluoses, hydroxybutylcelluloses and hydroxyphenylcelluloses, and hydroxyalkyl alkylcelluloses (e.g., hydroxypropyl methylcellulose), and mixtures thereof. The hydroxyalkylcelluloses comprise polymers having a 9,500 to 1,250,000 number-average molecular weight. For example, hydroxypropyl celluloses having number average molecular weights of between 80,000 to 850,000 are useful. The flow promoting layer may be prepared from conventional solutions or suspensions of the aforementioned materials in aqueous solvents or inert organic solvents. Preferred materials for the subcoat or flow promoting layer include hydroxypropyl cellulose, hydroxyethyl cellulose, hydroxypropyl methyl cellulose, povidone [poly(vinylpyrrolidone)], polyethylene glycol, and mixtures thereof. More preferred are mixtures of hydroxypropyl cellulose and povidone, prepared in organic solvents, particularly organic polar solvents such as lower alkanols having 1-8 carbon atoms, preferably ethanol, mixtures of hydroxyethyl cellulose and hydroxypropyl methyl cellulose prepared in aqueous solution, and mixtures of hydroxyethyl cellulose and polyethylene glycol prepared in aqueous solution. Most preferably, the flow-promoting layer consists of a mixture of hydroxypropyl cellulose and povidone prepared in ethanol. Conveniently, the weight of the flow-promoting layer applied to the bilayer core may be correlated with the thickness of the flow-promoting layer and residual drug remaining in a dosage form in a release rate assay such as described herein. During manufacturing operations, the thickness of the flow-promoting layer may be controlled by controlling the weight of the subcoat taken up in the coating operation. When the flow-promoting layer is formed as a subcoat, i.e., by coating onto the tableted bilayer composite drug layer and push layer, the subcoat can fill in surface irregularities formed on the bilayer core by the tableting process. The resulting smooth external surface facilitates slippage between the coated bilayer composite and the semipermeable wall during dispensing of the drug, resulting in a lower amount of residual drug composition remaining in the device at the end of the dosing period. When the flow-promoting layer is fabricated of a gel-forming material, contact with water in the environment of use facilitates formation of a gel or gel-like inner coat having a viscosity that may promote and enhance slippage between the semipermeable wall and the drug layer.

The wall is a semipermeable composition, permeable to the passage of an external fluid, such as water and biological fluids, and substantially impermeable to the passage of active agent, osmagent, osmopolymer and the like. The selectively semipermeable compositions used for forming the wall are essentially nonerodible and are insoluble in biological fluids during the life of the dosage form. The wall need not be semipermeable in its entirety, but at least a portion of the wall is semipermeable to allow fluid to contact or communicate with the push displacement layer such that the push layer can imbibe fluid and expand during use. The wall preferably comprises a polymer such as a cellulose acylate, cellulose diacylate, cellulose triacylate, including without limitation, cellulose acetate, cellulose diacetate, cellulose triacetate, or mixtures thereof The wall forming material may also be selected from ethylene vinyl acetate copolymers, polyethylene, copolymers of ethylene, polyolefins including ethylene oxide copolymers such as Engage® (DuPont Dow Elastomers), polyamides, cellulosic materials, polyurethanes, polyether blocked amides copolymers such as PEBAX® (Elf Atochem North America, Inc.), cellulose acetate butyrate, and polyvinyl acetate. Typically, the wall comprises 60 weight percent (wt %) to 100 wt % of the cellulosic wall-forming polymer, or the wall can comprise 0.01 wt % to 10 wt % of ethylene oxide-propylene oxide block copolymers, known as poloxamers, or 1 wt % to 35 wt % of a cellulose ether selected from the group consisting of hydroxypropylcellulose and hydroxypropylalkylcellulose and 5 wt % to 15 wt % of polyethylene glycol. The total weight percent of all components comprising the wall is equal to 100 wt %.

Representative polymers for forming the wall comprise semipermeable homopolymers, semipermeable copolymers, and the like. Such materials comprise cellulose esters, cellulose ethers and cellulose ester-ethers. The cellulosic polymers have a degree of substitution (DS) of their anhydroglucose unit of from greater than 0 up to 3, inclusive. Degree of substitution (DS) means the average number of hydroxyl groups originally present on the anhydroglucose unit that are replaced by a substituting group or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkysulfamate, semipermeable polymer forming groups, and the like, wherein the organic moieties contain from one to twelve carbon atoms, and preferably from one to eight carbon atoms.

The semipermeable compositions typically include a cellulose acylate, cellulose diacylate, cellulose triacylate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers include cellulose acetate having a DS of 1.8 to 2.3 and an acetyl content of 32 to 39.9%; cellulose diacetate having a DS of 1 to 2 and an acetyl content of 21 to 35%; cellulose triacetate having a DS of 2 to 3 and an acetyl content of 34 to 44.8%; and the like. More specific cellulosic polymers include cellulose propionate having a DS of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a DS of 1.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a DS of 2.6 to 3, such as cellulose trivalerate, cellulose trilamate, cellulose tripalmitate, cellulose trioctanoate and cellulose tripropionate; cellulose diesters having a DS of 2.2 to 2.6, such as cellulose disuccinate, cellulose dipalmitate, cellulose dioctanoate, cellulose dicaprylate, and the like; and mixed cellulose esters, such as cellulose acetate valerate, cellulose acetate succinate, cellulose propionate succinate, cellulose acetate octanoate, cellulose valerate palmitate, cellulose acetate heptanoate, and the like. Semipermeable polymers are known in U.S. Pat. No. 4,077,407, and they can be synthesized by procedures described in Encyclopedia of Polymer Science and Technology, Vol. 3, pp. 325-354, Interscience Publishers Inc., New York, N.Y. (1964).

Additional semipermeable polymers for forming the outer wall comprise cellulose acetaldehyde dimethyl acetate; cellulose acetate ethylcarbamate; cellulose acetate methyl carbamate; cellulose dimethylaminoacetate; semipermeable polyamide; semipermeable polyurethanes; semipermeable sulfonated polystyrenes; cross-linked selectively semipermeable polymers formed by the coprecipitation of an anion and a cation, as disclosed in U.S. Pat. Nos. 3,173,876; 3;276,586; 3,541,005; 3,541,006 and 3,546,142; semipermeable polymers, as disclosed by Loeb, et al. in U.S. Pat. No. 3,133,132; semipermeable polystyrene derivatives; semipermeable poly(sodium styrenesulfonate); semipermeable poly(vinylbenzyltrimethylammonium chloride); and semipermeable polymers exhibiting a fluid permeability of 10⁻⁵ to 10⁻² (cc. mil/cm hr. atm), expressed as per atmosphere of hydrostatic or osmotic pressure differences across a semipermeable wall. The polymers are known to the art in U.S. Pat. Nos. 3,845,770; 3,916,899 and 4,160,020; and in Handbook of Common Polymers, Scott and Roff, Eds., CRC Press, Cleveland, Ohio (1971).

The wall may also comprise a flux-regulating agent. The flux regulating agent is a compound added to assist in regulating the fluid permeability or flux through the wall. The flux-regulating agent can be a flux-enhancing agent or a flux-decreasing agent. The agent can be preselected to increase or decrease the liquid flux. Agents that produce a marked increase in permeability to fluid such as water are often essentially hydrophilic, while those that produce a marked decrease to fluids such as water are essentially hydrophobic. The amount of regulator in the wall when incorporated therein generally is from about 0.01% to 20% by weight or more. The flux regulator agents may include polyhydric alcohols, polyalkylene glycols, polyalkylenediols, polyesters of alkylene glycols, and the like. Typical flux enhancers include polyethylene glycol 300, 400, 600, 1500, 4000, 6000 and the like; low molecular weight glycols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glycol dipropionate, glycerol acetate esters, and the like. Presently preferred flux enhancers include the group of difunctional block-copolymer polyoxyalkylene derivatives of propylene glycol known as poloxamers (BASF). Representative flux-decreasing agents include phthalates substituted with an alkyl or alkoxy or with both an alkyl and alkoxy group such as diethyl phthalate, dimethoxyethyl phthalate, dimethyl phthalate, and [di(2-ethylhexyl)phthalate], aryl phthalates such as triphenyl phthalate, and butyl benzyl phthalate; insoluble salts such as calcium sulfate, barium sulfate, calcium phosphate, and the like; insoluble oxides such as titanium oxide; polymers in powder, granule and like form such as polystyrene, polymethylmethacrylate, polycarbonate, and polysulfone; esters such as citric acid esters esterified with long chain alkyl groups; inert and substantially water impermeable fillers; resins compatible with cellulose based wall forming materials, and the like.

Other materials that may be included in the semipermeable wall material for imparting flexibility and elongation properties to the wall, for making the wall less brittle to nonbrittle and to render tear strength. Suitable materials include phthalate plasticizers such as dibenzyl phthalate, dihexyl phthalate, butyl octyl phthalate, straight chain phthalates of six to eleven carbons, di-isononyl phthalate, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a wall when incorporated therein is about 0.01% to 20% weight, or higher.

Manufacture of Dosage Forms

In brief, the dosage forms are manufactured using the following basic steps, which are discussed in greater detail below. The core can in principle include multiple drug layers and multiple push displacement layers, although the ascending rate of release can be obtained using only a single drug layer and single push displacement layer. Optionally, the ratio of the drug layer and the push layer can be adjusted to provide for a greater or lesser rate of release of the drug layer from the core. Thus, the addition of a greater amount of push displacement layer into the dosage form can provide an ascending release rate for even longer release periods, of greater than about 8-10 hours.

The core is formed first and coated with the flow-promoting layer; the coated core can then be dried, though this is optional; and the semipermeable wall is then applied. An orifice is then provided by a suitable procedure (e.g., laser drilling), although alternative procedures can be used which provide an orifice which is formed at a later time (a formable orifice). Finally, the finished dosage forms are dried and are ready for use or for coating with an immediate release drug coating.

The drug layer is formed as a mixture containing the nonopioid analgesic, the opioid analgesic and the binding agent and other ingredients. The drug layer can be formed from particles by comminution that produces the size of the drug and the size of the accompanying polymer used in the fabrication of the drug layer, typically as a core containing the compound, according to the mode and the manner of the invention. The means for producing particles include granulation, spray drying, sieving, lyophilization, crushing, grinding, jet milling, micronizing and chopping to produce the intended micron particle size. The process can be performed by size reduction equipment, such as a micropulverizer mill, a fluid energy grinding mill, a grinding mill, a roller mill, a hammer mill, an attrition mill, a chaser mill, a ball mill, a vibrating ball mill, an impact pulverizer mill, a centrifugal pulverizer, a coarse crusher and a fine crusher. The size of the particle can be ascertained by screening, including a grizzly screen, a flat screen, a vibrating screen, a revolving screen, a shaking screen, an oscillating screen and a reciprocating screen. The processes and equipment for preparing the drug and binding agent are disclosed in Pharmaceutical Sciences, Remington, 17th Ed., pp. 1585-1594 (1985); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Hixon, pp. 94-103 (1990).

Exemplary solvents suitable for manufacturing the respective walls, layers, coatings and subcoatings utilized in the dosage forms of the invention comprise aqueous and inert organic solvents that do not adversely harm the materials utilized to fabricate the dosage forms. The solvents broadly include members selected from the group consisting of aqueous solvents, alcohols, ketones, esters, ethers, aliphatic hydrocarbons, halogenated solvents, cycloaliphatics, aromatics, heterocyclic solvents and mixtures thereof. Typical solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride nitroethane, nitropropane tetrachloroethane, ethyl ether, isopropyl ether, cyclohexane, cyclooctane, benzene, toluene, naphtha, 1,4-dioxane, tetrahydrofuran, diglyme, water, aqueous solvents containing inorganic salts such as sodium chloride, calcium chloride, and the like, and mixtures thereof such as acetone and water, acetone and methanol, acetone and ethyl alcohol, methylene dichloride and methanol, and ethylene dichloride and methanol.

Pan coating may be conveniently used to provide the completed dosage form, except for the exit orifice. In the pan coating system, the subcoat of the wall-forming compositions can be deposited by successive spraying of the respective composition on the bilayered core comprising the drug layer and the push layer accompanied by tumbling in a rotating pan. A pan coater can be used because of its availability at commercial scale. Other techniques can be used for coating the drug core. The coated dosage form can be dried in a forced-air oven, or in a temperature and humidity controlled oven to free the dosage form of solvent. Drying conditions will be conventionally chosen on the basis of available equipment, ambient conditions, solvents, coatings, coating thickness, and the like.

Other coating techniques can also be employed. For example, the semipermeable wall and the subcoat of the dosage form can be formed in one technique using the air-suspension procedure. This procedure consists of suspending and tumbling the bilayer core in a current of air, an inner subcoat composition and an outer semipermeable wall forming composition, until, in either operation, the subcoat and the outer wall coat is applied to the bilayer core. The air-suspension procedure is well suited for independently forming the wall of the dosage form. The air-suspension procedure is described in U.S. Pat. No. 2,799,241; in J. Am. Pharm. Assoc., Vol. 48, pp. 451-459 (1959); and, ibid., Vol. 49, pp. 82-84 (1960). The dosage form also can be coated with a Wurster® air-suspension coater using, for example, methylene dichloride methanol as a cosolvent. An Aeromatic® air-suspension coater can be used employing a cosolvent.

The dosage form of the invention may be manufactured by standard techniques. For example, the dosage form may be manufactured by the wet granulation technique. In the wet granulation technique, the drug and the ingredients comprising the first layer or drug composition are generally blended using an organic solvent, such as denatured anhydrous ethanol, as the granulation fluid. The ingredients forming the first layer or drug composition are individually passed through a preselected screen and then thoroughly blended in a mixer. Next, other ingredients comprising the first layer can be dissolved in a portion of the granulation fluid, such as the solvent described above. Then, the latter prepared wet blend is slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven. The dried granules are then sized. Next, magnesium stearate is added to the drug granulation, then put into milling jars and mixed on a jar mill for 10 minutes. The composition is pressed into a layer, for example, in a Manesty® press. The speed of the press is set at 20 rpm and the maximum load set at 2 tons. The first layer is pressed against the composition forming the second layer and the bilayer tablets are fed to the Kilian® Dry Coater press and surrounded with the drug-free coat, followed by the exterior wall solvent coating.

In another manufacture, the active agents (e.g., a nonopioid analgesic and opioid analgesic) and other ingredients comprising the first layer facing the exit means are blended and pressed into a solid layer. The layer possesses dimensions that correspond to the internal dimensions of the area the layer is to occupy in the dosage form, and it also possesses dimensions corresponding to the second layer for forming a contacting arrangement therewith. The drug and other ingredients can also be blended with a solvent and mixed into a solid or semisolid form by conventional methods, such as ballmilling, calendering, stirring or rollmilling, and then pressed into a preselected shape. Next, the expandable layer, e.g., a layer of osmopolymer composition, is placed in contact with the layer of drug in a like manner. The layering of the drug formulation and the osmopolymer layer can be fabricated by conventional two-layer press techniques. The two contacted layers are first coated with the flow-promoting subcoat and then an outer semipermeable wall. The air-suspension and air-tumbling procedures comprise in suspending and tumbling the pressed, contacting first and second layers in a current of air containing the delayed-forming composition until the first and second layers are surrounded by the wall composition.

Another manufacturing process that can be used for providing the compartment-forming composition comprises blending the powdered ingredients in a fluid bed granulator. After the powdered ingredients are dry blended in the granulator, a granulating fluid, for example, poly(vinylpyrrolidone) in water, is sprayed onto the powders. The coated powders are then dried in the granulator. This process granulates all the ingredients present therein while adding the granulating fluid. After the granules are dried, a lubricant, such as stearic acid or magnesium stearate, is mixed into the granulation using a tote or V-blender. The granules are then pressed in the manner described above.

The flow-promoting layer is then applied to the pressed cores. The semipermeable wall is coated onto the outer surface of the pressed core and/or flow promoting layer. The semi-permeable wall material is dissolved in an appropriate solvent such as acetone or methylene chloride and is then applied to the pressed shape by molding, air spraying, dipping or brushing a solvent-based solution of the wall material onto the shape, as described in U.S. Pat. Nos. 4,892,778 and 4,285,987. Other methods for applying the semi-permeable wall include an air suspension procedure, where the pressed shape is suspended and tumbled in a current of air and wall forming material as described in U.S. Pat. No. 2,799,241, and a pan coating technique.

After application of the semi-permeable wall to the pressed shape, a drying step is generally required and, then, suitable exit means for the active agent must be formed through the semi-permeable membrane. Depending on the properties of the active agent and other ingredients within the cavity and the desired release rate for the dosage form, one or more orifices for active agent delivery are formed through the semi-permeable membrane by mechanical drilling, laser drilling, or the like.

The exit orifice can be provided during the manufacture of the dosage form or during drug delivery by the dosage form in a fluid environment of use. The expression “exit orifice” as used for the purpose of this invention includes a passageway; an aperture; an orifice; or a bore. The orifice may range in size from a single large orifice encompassing substantially an entire surface of the dosage form to one or more small orifices selectively located on the surface of the semi-permeable membrane. The exit orifice can have any shape, such as round, triangular, square, elliptical and the like for the release of a drug from the dosage form. The dosage form can be constructed with one or more exits in spared apart relation or one or more surfaces of the dosage form.

The exit orifice may be from 10% to 100% of the inner diameter of the compartment formed by the wall, preferably from 30% to 100%, and most preferably from 50% to 100%. In preferred embodiments, the drug layer is released from the dosage form as an erodible solid through a relatively large orifice of a size of at least 100 mils to 100% of the inner diameter of the compartment formed by the wall, typically from about 125 mils (thousandths of an inch) to about 185 mils, or from about 3.175 to about 4.7 mm. The use of a smaller orifice may be employed if desired to provide a further delay in release of the drug layer.

The exit orifice can be performed by drilling, including mechanical and laser drilling, through the outer coat, the inner coat, or both. Exits and equipment for forming exits are disclosed in, for example, U.S. Pat. Nos. 3,845,770 and 3,916,899 to Theeuwes and Higuchi; in U.S. Pat. No. 4,063,064 to Saunders, et al.; and in U.S. Pat. No. 4,088,864 to Theeuwes, et al.

The exit can also be an orifice that is formed from a substance or polymer that erodes, dissolves or is leached from the outer coat or wall or inner coat to form an exit orifice, as disclosed, for example, in U.S. Pat. Nos. 4,200,098 and 4,285,987. Representative materials suitable for forming an orifice, or a multiplicity of orifices comprise leachable compounds, such as a fluid removable pore-former such as inorganic and organic salts, inorganic or organic oxides, carbohydrates, polymers, such as leachable poly(glycolic) acid or poly(lactic) acid polymers, gelatinous filaments, poly(vinyl alcohol), leachable polysaccharides, sugars such as sorbitol, which can be leached from the wall. For example, an exit, or a plurality of exits, can be formed by leaching sorbitol, lactose, fructose, glucose, mannose, galactose, talose, sodium chloride, potassium chloride, sodium citrate and mannitol from the wall.

In addition, in some embodiments, the osmotic dosage form can be in the form of an extruded tube open at one or both ends, as described in commonly owned U.S. Pat. No. 6,491,683 to Dong, et al. In the extruded tube embodiment, it is not necessary to provide an additional exit means.

Active Agents

A wide variety of active agents may be used in the dosage forms. The dosage forms described herein are particularly useful for providing an ascending rate of release of active agents, which can be particularly desirable when the active agents are metabolized or neutralized quickly, or where tolerance develops. The dosage forms are also useful for providing sustained release of difficult to formulate or poorly soluble active agents, especially when large doses of these agents are required to be delivered over a prolonged period of time, or at an ascending rate over a prolonged period of time. The dosage forms are also useful for providing sustained release and prolonged delivery of combinations of active agents, and can provide for the proportional delivery of different active agents even when there is a great disparity in solubility between the active agents.

The active agents that can be delivered by the controlled release dosage form comprise inorganic and organic active agents. The active agents include active agents that act on peripheral nerve, adrenergic receptors, cholinergic receptors, the central nervous system, skeletal muscles, the cardiovascular system, smooth muscles, the blood circulatory system, synaptic sites, neuroeffector junctional sites, the endocrine system, hormone systems, the immunological system, organ systems, body passageways, reproductive systems, the skeletal system, autocoid systems, alimentary and excretory systems inhibitors of autocoids and histamine systems, without limitation. The active agents that can be delivered for acting on these recipients include anticonvulsants, analgesics, anti-diabetic agents, anti-parkinson agents, anti-inflammatory agents, anesthetics, antimicrobial agents, antimalarials, antiparasitic agents, antihypertensive agents, angiotensin converting enzyme inhibitors, antihistamines, antipyretics, alpha-adrenergic receptor agonists, alpha-adrenergic receptor blockers, biocides, bactericides, bronchial dilators, beta-adrenergic stimulators, beta-adrenergic blocking drugs, contraceptives, cardiovascular drugs, calcium channel blockers, depressants, diagnostic agents, diuretics, electrolytes, hypnotics, hormonal agents, steroids, antihyperglycemics, muscle contractants, muscle relaxants, ophthalmics, psychic energizers, parasympathomimetics, sedatives, selective androgen receptor modulators, selective estrogen receptor inhibitors, sympathomimetics, tranquilizers, urinary tract drugs, vaginal drugs, and vitamins. Active agents can be included in the sustained release dosage form in free base form, or as a salts, acids, amides, esters, polymorphs, solvates, hydrates, dehydrates, co-crystals, anhydrous, or amorphous forms thereof.

Suitable active agents may be selected from, for example, proteins, enzymes, enzyme inhibitors, hormones, polynucleotides, nucleoproteins, polysaccharides, glycoproteins, lipoproteins, polypeptides, steroids, hypnotics and sedatives, psychic energizers, tranquilizers, anticonvulsants, antidepressants, muscle relaxants, antiparkinson agents, analgesics, anti-inflammatories, antihystamines, local anesthetics, muscle contractants, antimicrobials, antimalarials, antivirals, antibiotics, antiobesity agents, hormonal agents including contraceptives, sympathomimetics, polypeptides and proteins capable of eliciting physiological effects, diuretics, lipid regulating agents, antiandrogenic agents, antiparasitics, neoplastics, antineoplastics, antihyperglycemics, hypoglycemics, nutritional agents and supplements, growth supplements, fats, ophthalmics, antienteritis agents, electrolytes and diagnostic agents.

Examples of particular active agents useful in this invention are not particularly limiting. Without attempting to name every agent that can be used, the active agents can include prochlorperazine edisylate, ferrous sulfate, albuterol, aminocaproic acid, mecamylamine hydrochloride, procainamide hydrochloride, amphetamine sulfate, methamphetamine hydrochloride, benzphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, triethylperazine maleate, anisindione, diphenadione erythrityl tetranitrate, digoxin, isoflurophate, acetazolamide, nifedipine, methazolamide, bendroflumethiazide, chlorpropamide, glipizide, glyburide, gliclazide, tobutamide, chlorproamide, tolazamide, acetohexamide, metformin, troglitazone, orlistat, bupropion, nefazodone, tolazamide, chlormadinone acetate, phenaglycodol, allopurinol, aluminum aspirin, methotrexate, acetyl sulfisoxazole, hydrocortisone, hydrocorticosterone acetate, cortisone acetate, dexamethasone and its derivatives such as betamethasone, triamcinolone, methyltestosterone, 17-.beta.-estradiol, ethinyl estradiol, ethinyl estradiol 3-methyl ether, prednisolone, 17-.beta.-hydroxyprogesterone acetate, 19-norprogesterone, norgestrel, norethindrone, norethisterone, norethiederone, progesterone, norgesterone, norethynodrel, terfandine, fexofenadine, aspirin, acetaminophen, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, selegiline, chlorpromazine, methyldopa, dihydroxyphenylalanine, calcium gluconate, ketoprofen, ibuprofen, cephalexin, erythromycin, haloperidol, zomepirac, ferrous lactate, vincamine, phenoxybenzamine, diltiazem, milrinone, captropril, mandol, quanbenz, hydrochlorothiazide, ranitidine, flurbiprofen, fenbufen, fluprofen, tolmetin, alclofenac, mefenamic, flufenamic, difuninal, nimodipine, nitrendipine, nisoldipine, nicardipine, felodipine, lidoflazine, tiapamil, gallopamil, amlodipine, mioflazine, lisinopril, enalapril, captopril, ramipril, enalaprilat, famotidine, nizatidine, sucralfate, etintidine, tetratolol, minoxidil, chlordiazepoxide, diazepam, amitriptyline, and imipramine, and pharmaceutical salts of these active agents. Further examples are proteins and peptides which include, but are not limited to, insulin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, bovine somatotropin, porcine somatropin, oxytocin, vasopressin, desmopressin, prolactin, somatostatin, lypressin, pancreozymin, luteinizing hormone, LHRH, interferons, interleukins, growth hormones such as human growth hormone, bovine growth hormone and porcine growth hormone, fertility inhibitors such as the prostaglandins, fertility promoters, growth factors, and human pancreas hormone releasing factor.

Active agents in the field of antidepressants may be selected from the group consisting of tertiary amine tricyclics such as, for example, amitriptyline, clomipramine, doxepin, imipramine, (+)-trimipramine; secondary amine tricyclics such as, for example, amozapine, desipramine, maprotiline, nortiriptyline, protryptilyline; serotonin re-uptake inhibitors such as, for example, fluoxetine, fluvoxamine, paroxetine, sertraline, venlafazine; and atypical antidepressants such as brupropion, nefazodone, trazodone, phenelzine, tranylcypromime, selegiline, and pharmaceutically acceptable salts thereof. The dosage form typically may include a carrier, e.g., hydrophilic polymer, in a composition with the active agent.

Factors to consider in preparing a particular dosage form are the half life of the drug in the plasma of a patient, the relative bioavailability and absorption of a particular drug in the upper and lower GI tract, whether tolerance develops to a given dose of a drug, whether drug incompatibilities, synergism or interactions occur, the dose required to maintain a particular plasma profile, and the like.

For example, nonsteroidal anti-inflammatory agents or nonopioid analgesics can be delivered using the sustained release dosage forms over a prolonged period of time, enabling a less frequent dosing regimen, such as twice a day dosing, or once a day dosing for active agents having a long half life in plasma. Additional active agents can be included with the nonsteroidal anti-inflammatory agent, for example, for gastric protection. Gastric protective agents include histamine H₂-receptor antagonists (e.g., cimetidine, ranitidine, famotidine, or nizatidine), cytoprotective agents (e.g., misoprostol, rebamipide, ecabet, or carbenoxolone), or proton pump inhibitors (e.g., for example as disclosed in EP-A1-0005129, EP-A1-174 726, EP-A1-166 287, GB 2 163 747 and WO90/06925, WO91/19711, WO91/19712, WO95/01977, WO94/27988, and U.S. Pat. No. 6,610,323 to Lundberg, for example, without limitation alpha-pyridylmethylsulfinyl benzimidazoles such as lansoprazole, omeprazole, rabeprazole, pantoprazole, or esomeprazole).

5-HT-agonists can be included in a dosage form for delivery of NSAIDS for treatment of migraine, for example. 5-HT-agonists include, without limitation, indole derivatives such as triptans, including but not limited to, sumatriptan, eletriptan (described in European Patent Application 379314), Allelix ALX 1323, rizatriptan, frovatriptan, almotriptan, zolmitriptan and naratriptan, such as described in U.S. Pat. No. 4,816,470; ergot alkaloids such as ergotamine (e.g., ergotamine tartrate), dihydroergotamine, bromocriptine, ergonovine and methyl ergonovine (e.g., ergonovine maleate), methysergide, and ergoloid mesylates, including dihydroergocornine, dihydroergocristine, dihydroergocryptine (alpha and beta), and dihydroergotamine mesylate (DHE 45), and as described in U.S. Pat. No. 6,586,458 to Plachetka.

Antibiotics can also be formulated for delivery using the sustained release dosage forms described herein. Any antibiotic that can be administered orally can be included in the controlled release dosage form. Antibiotics include anti-protozoal agents; anti-helminthic agents; agents effective against bacterial species, including gram-positive and gram-negative cocci, gram-positive and gram-negative bacilli, acid-fast bacilli, spirochetes, actinomycetes; species of fungi, such as candida, histoplasma, paracoccidioides, sporothrix, aspergilli, mucormycoses, cryptococci; viruses; as well as miscellaneous organisms such as ureaplasma, mycoplasma, rickettsia, chlamydia, pneumocystis. Exemplary antibiotics include erythromycin, amoxicillin, clarithromycin, tetracycline, or metronidazole. Antibiotics that are poorly soluble, insoluble or poorly dissolving are ideally delivered using the dosage forms described herein. For example, erythromycin is typically required in one or more oral doses of 250 mg (or more) taken four times a day for a total daily dose of 1-2 grams per day. Doses as high as 8 grams per day have been prescribed.

The dosage forms are particularly well suited for the formulation and delivery of poorly soluble compounds such as topiramate, ibuprofen, acetaminophen, gemfibrozil, and the like. The dosage forms can be advantageously used to provide sustained release formulations of nonopioid analgesic agents (particularly acetaminophen) or nonsteroidal anti-inflammatory agents (e.g., ibuprofen, ketoprofen) due to the large doses of these agents needed and the difficulty in formulating and delivering these agents to a patient in need of treatment. In this regard, the combination of opioid analgesics and nonopioid analgesics is a preferred embodiment of dosage forms described herein.

Nonopioid analgesics include the class of compounds known as nonsteroidal anti-inflammatory agents. Examples of nonopioid analgesics include the poorly soluble para-aminophenol derivatives exemplified by acetaminophen, aminobenzoate potassium, aminobenzoate sodium, but can also include salicylic acid derivatives such as aspirin, sulfasalazine, salicylamide, sodium salicylate, and salicylate potassium; aryl propionic acids including benoxaprofen, decibuprofen, flurbiprofen, fenoprofen, ibuprofen, indoprofen, ketoprofen, naproxen, naproxol, oxaprozin; heteroaryl acetic acids such as diclofenac, ketorolac, tolmetin; indole and indene acetic acids including indomethacin, sulindac; selective COX-2 inhibitors such as celecoxib, rofecoxib, valdecoxib, etodolac, ibufenac, nimesulfide, JTE-522, L-745,337, or NS398; alkanones such as nabumetone; oxicams including meloxicam, piroxicam, lornoxicam, cinnoxicam, sudoxicam, tenoxicam; anthranilic acids such as mefenamic acid and meclofenamic acid. Preferred nonopioid analgesic agents include acetaminophen and ibuprofen. The amount of nonopioid analgesic agent in a single dosage form is typically 0.5 mg to 1000 mg, and more typically between about 200 and about 600 mg.

The active agent can also be an opioid analgesic. Representative opioid analgesics include without limitation alfentanil, allylprodine, alphaprodine, anileridne, benzylmorphine bezitramide, buprenorphine, butorphanol, clonitazene, codeine, cyclazocine, desomorphine, dextromoramide, dezocine, diampromide, dihydrocodeine, dihydromorphine, dimenoxadol, diepheptanol, dimethylthiambutene, dioxaphetyl butyrate, dipipanone, eptazone, ethoheptazine, ethylmethylthiambutene, ethylmorphine, propylmorphine, etonitazene, fentanyl, heroin, hydrocodone, hydromorphone, hydroenitabas, hydrocypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphone, phenazocine, phenoperidine, piminodine, pirtramide, propheptazine, promedol, properidine, propiram, propoxyphene, sufentanil, tramadol, and tilidine. The dose of opioid drug 14 is 0.1 μg to 700 mg.

Methods of Use

The dosage forms described above can be used in a variety of methods. For example, the dosage forms can be used in methods for providing an effective concentration of an active agent (e.g., an opioid analgesic and nonopioid analgesic) in the plasma of a human patient for the treatment of a disorder or condition. The dosage forms can also be used in methods for providing sustained release of an active agent and delivery to the gastrointestinal tract of a human patient. In particular embodiments, the dosage forms can be used in methods for treating pain in a human patient, for example, in providing an effective amount of an analgesic composition for treating pain, and so forth.

The dosage forms are particularly useful for providing sustained release of poorly soluble or insoluble pharmaceutically active agents, particularly when the active agents are used in combination with additional active agents. The dosage forms provide release of the active agents at ascending release rates, and the release rates can be proportional to each other, providing a unique ability to tailor the plasma concentration in the patient to either parallel plasma concentrations or differing plasma concentrations, such as would occur if one agent is metabolized at a slower rate than the other active agent. The active agents can be chosen so that their rates of inactivation or excretion are similar, thus providing a parallel plasma profile, or so that their rates of inactivation or excretion are different, thus providing a plasma profile that diverges.

In addition, in the event that tolerance or desensitization to a particular active agent occurs, an ascending release rate provides a means of overcoming the difficulty in maintaining effective therapeutic levels of the active agent. Thus, for any decrease in efficacy due to the development of tolerance or to slow dissociation rates from inhibitory receptors, the increasing plasma concentrations provide a means of compensating for any reduced efficacy of the active agent, even under circumstances where target receptors in the patient have become less sensitive to the active agent.

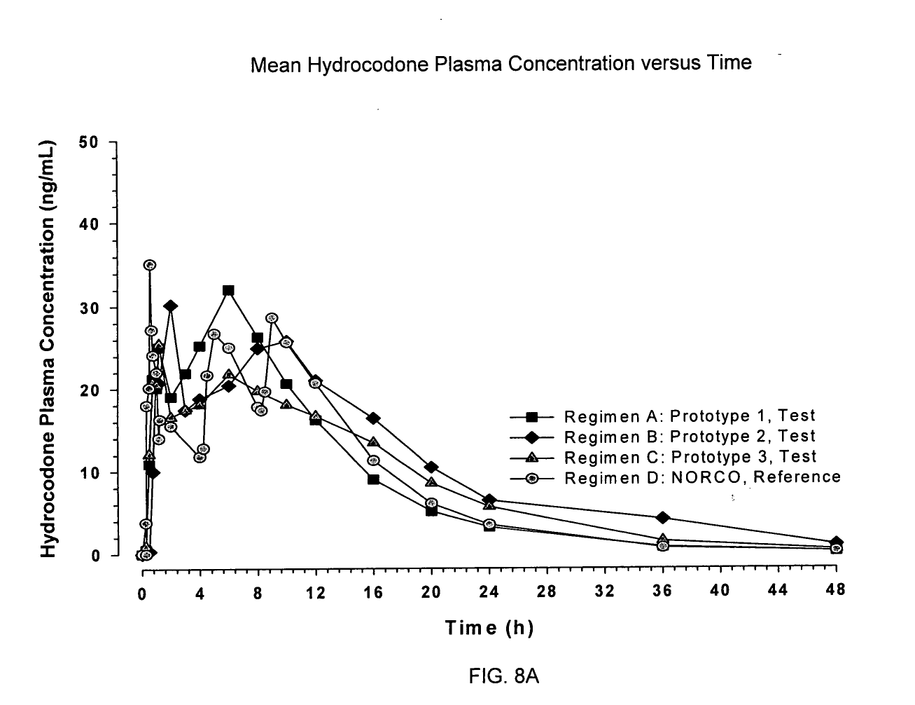

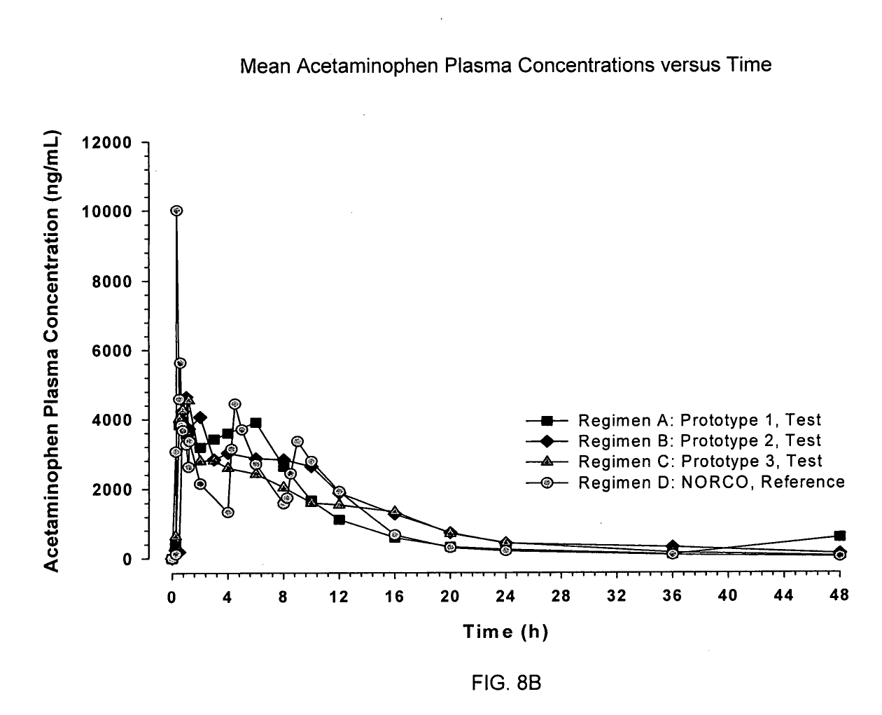

As shown in FIGS. 8A and B, and discussed in greater detail below, three different ascending release rates for hydrocodone produced varying ascending plasma profiles in human patients, while the same ascending release rates for acetaminophen produced either an ascending, zero order or descending plasma profile in human patients. Thus the plasma profile of the active agent appears to be exquisitely sensitive to both the release rate and the rate of metabolic inactivation of the active agent.

As described in detail in Example 3, a clinical trial was performed to determine the bioavailability of the sustained release dosage forms described herein, as well as their bioequivalence to an immediate release dosage form dosed every four hours ((NORCO®). The pharmacokinetic parameters produced in human patients are presented in detail in ALZ5130, filed on even date herewith, the disclosure of which is hereby incorporated by reference in its entirety.

In this clinical study, bioavailability of several representative dosage forms and their bioequivalence with an immediate release dosage form (NORCO®, 1 tablet every 4 hours for 3 doses) was demonstrated. Dosage forms having a variety of release rates, producing T₉₀'s of approximately 6, 8 and 10 hours, were tested. FIGS. 8A and B illustrate the comparison between the mean in vivo plasma profiles of hydrocodone and acetaminophen observed after administration of representative dosage forms having T₉₀'s of approximately 6, 8 and 10 hours, and after administration of the immediate release dosage form comprising acetaminophen and hydrocodone bitartrate every four hours. As these figures illustrate, volunteers receiving two tablets of each of the three dosage forms prepared according the procedure of Example 1 exhibited a rapid rise in plasma concentrations of hydrocodone and acetaminophen after oral administration at time zero. The dosage forms produced a rapid rise in plasma levels of hydrocodone and acetaminophen, followed by a sustained release of hydrocodone and acetaminophen sufficient to provide therapeutically effective levels in the plasma of the patients for an extended period of time, suitable for twice daily dosing.

All three of the dosage forms in Regimens A, B and C produced an ascending plasma profile of hydrocodone (see FIG. 8A), while only Regimen A produced an ascending plasma profile of acetaminophen. Regimens B and C, with their slower rate of release of drug, provided acetaminophen at a rate that produced a zero order or even descending plasma profile of acetaminophen, due to the rapid metabolism of this drug. Thus depending on the pharmacokinetic properties of the drug and the individual patient's metabolism, an ascending rate of release of drug in vitro can manifest in vivo as an ascending, zero order or descending plasma profile.

It is to be understood that while the invention has been described in conjunction with the preferred specific embodiments thereof, that the description above as well as the examples that follow are intended to illustrate and not limit the scope of the invention. The practice of the present invention will employ, unless otherwise indicated, conventional techniques of organic chemistry, polymer chemistry, pharmaceutical formulations, and the like, which are within the skill of the art. Other aspects, advantages and modifications within the scope of the invention will be apparent to those skilled in the art to which the invention pertains. Such techniques are explained fully in the literature.

All patents, patent applications, and publications mentioned herein, both supra and infra, are hereby incorporated by reference.

In the following examples, efforts have been made to ensure accuracy with respect to numbers used (e.g., amounts, temperature, etc.) but some experimental error and deviation should be accounted for. Unless indicated otherwise, temperature is in degrees ° C. and pressure is at or near atmospheric. All solvents were purchased as HPLC grade.

Abbreviations:

APAP: acetaminophen
AUC: area under the plasma concentration-time curve
HBH: hydrocodone bitartrate
HC: hydrocodone
HEC: hydroxyethylcellulose
HM: hydromorphone
HPMC: hydroxypropylmethylcellulose
HPC: hydroxypropylcellulose
PEO: poly(ethylene oxide)
PVP: polyvinylpyrrolidone

### EXAMPLE 1

A dosage form containing 500 mg acetaminophen and 15 mg hydrocodone was prepared using procedures as follows:

Preparation of the Drug Layer Granulation

A twenty five kilogram lot of the drug layer was granulated using the medium fluid bed granulator (mFBG). A 5% manufacturing excess of hydrocodone bitartrate (HBH) was added to maintain target drug amounts in the compressed cores as established during the experimental scale up work. The binder solution was prepared by dissolving the povidone in purified water making a 7.5 wt % solution.

The specified amounts of APAP, polyethylene oxide 200 K (polyox N-80), croscarmellose sodium (Ac-di-sol), and poloxamer 188 were charged into the FBG bowl. The bed was fluidized and the binder solution was sprayed immediate thereafter. After 1000 g of the binder solution had been metered into the bowl, the granulation process was stopped the preweighed HBH was then charged into the bowl by placing it in a hole in the granulation and covering it up. The technique was employed to minimize the amount of drug that was lost through the filter bags. After a predetermined amount of binder solution had been sprayed, the spray was turned off and the granulation was dried until target moisture content was achieved. The granulation was then milled using a Fluid Air Mill fitted with a 10-mesh screen and using 2250-rpm milling rate.

Milled BHT was then added to replace the BHT lost from the polyethylene oxide and poloxamer in the granulation during processing. BHT is required in the polyethylene oxide and poloxamer to maintain viscosity. The raw material was hand sieved through a 40-mesh screen. The appropriate amount of BHT was dispersed into the top of the granulation in the blender using the Gemco blender, the mixture was blended fro 10 minutes, followed by the blending of the stearic acid and magnesium stearate in the granulation, using the same blender for 1 minute. The stearic acid and magnesium stearate were sized through a 40-mesh screen before being blended to the material in the blender. They were added to facilitate the ejection of the cores from the dies during core compression.

Preparation of the Osmotic Push Layer Granulation

Agglomerates of sodium chloride (NaCl) and ferric oxide were milled through the Quadro Comil fitted with a 21-mesh screen. The specified amounts of polyethylene oxide, milled NaCl, and milled ferric oxide were layered into the tote. Approximately half of the polyethylene oxide was on the bottom and the rest of the materials were in the middle. The remaining polyethylene oxide was on top. This sandwiching effect prevents the NaCl from re-agglomerating. Povidone was dissolved in purified water to make a binder solution with 13% solids. The appropriate amount of binder solution was prepared to make the granulation.

The dry ingredients in the tote were charged into the FBG bowl. The bed was fluidized, and the binder solution was sprayed as soon as the desired inlet air temperature was achieved. The fluidization airflow was increased by 500 m³/h for approximately every 3 minutes of spraying until the maximum airflow of 4000³/h was reached. After a predetermined amount of binder solution had been sprayed (48.077 kg), the spray was turned off and the granulation was dried to the target moisture content. The granulation was then milled into a 1530 L tote using a Fluid Air Mill fitted with a 7-mesh screen.

Milled BHT was added to prevent degradation of the polyethylene oxide and poloxamer granulation. The raw material was hand sieved through a 40-mesh screen. The appropriate amount of BHT was then dispersed into the top of the granulation in the tote. Using a tote tumbler, the mixture was blended for 10 minutes at 8 rpm, followed by the blending of the stearic acid in the granulation using a tote tumbler for 1 minute at 8 rpm. The stearic acid was sized through a 40-mesh screen before being blended to the material in the tote. It was added to facilitate the ejection of the tablets from the dies during compression.

Bilayer Core Compression

The drug layer granulation and the osmotic push granulation were compressed into bilayer cores using standard compression procedures. The Korsch press was used to manufacture the bilayer longitudinally compressed tablets (LCT). The press was set up with ¼ inch LCT punches and dies with round, deep concave punches and dies. The granulations were scooped into the hoppers leading to the appropriate location or station in the press. The appropriate amount of the drug layer granulation was added to the dies and was lightly tamped on the first compression station of the press. The push granulation was then added and the tablets were compressed to the final tablet thickness under the main compression roll on the second station of the press.

The initial adjustment of the tableting parameters (drug layer) is performed to produce cores with a uniform target drug layer weight of 413 mg containing typically 330 mg of APAP and 10 mg hydrocodone in each tablet. The second layer adjustment (osmotic push layer) of the tableting parameters is performed which bonds the drug layer to the osmotic layer to produce cores with a uniform final core weight, thickness, hardness, and friability. The foregoing parameters can be adjusted by varying the fill space and/or the force setting.

To control the tablet weight, the press has an automatic fill controller, based on compression force, which adjusts the fill quantity of granulation by changing the fill depth in the dies. The compression force and press speed were adjusted as necessary to manufacture tablets with satisfactory properties. The drug layer target weight was 413 mg and the push layer target weight was 138 mg. The pre-compression force was 60 N, adjusted as necessary to obtain quality cores, and the final compression was 6000 N, also adjusted as necessary. The press speed was 13 rpm and there were 14 stations.

Preparation of the Subcoat Solution and Subcoated System

The compressed cores were coated to a target subcoat weight of 17 mg/core. The subcoating solution contained 6 wt % solids and was prepared in a stainless steel mixing vessel. The solids (95% hydroxyethyl cellulose NF and 5% polyethylene glycol 3350) were dissolved in 100% water. The appropriate amount of water was first transferred into the mixing vessel. While mixing the water, the appropriate amount of polyethylene glycol was charged into the mixing vessel followed by the hydroxyethylcellulose. The materials were mixed together in the vessel until all the solids were dissolved.

A Vector Hi-Coater was used for the coating procedure. The coater was started, and after the target exhaust temperature was attained, the bilayer cores (nominally 9 kg per lot) were placed into the coater. The coating solution was sprayed immediately thereafter onto the rotating tablet bed. At regular intervals throughout the coating process, the weight gain was determined. When the desired wet weight gain was achieved (17 mg per core), the coating process was stopped.

Preparation of the Rate Controlling Membrane and Membrane Coated System

The membrane coating solution contained cellulose acetate 398-10 and poloxamer 188 in varying proportions to obtain a desired water permeation rate into the bilayer cores, and was coated onto the cores to a desired weight gain as described in A, B and C below. Weight gain may be correlated with T₉₀ for membranes of varying thickness in the release rate assay. When a sufficient amount of solution has been applied, conveniently determined by attainment of the desired membrane weight gain for a desired T₉₀s the membrane coating process was stopped.

The coating solution contained 5 wt % solids and was prepared in a 20 gallon closed jacketed stainless steel mixing vessel. The solids (75% cellulose acetate 398-10 and 15% poloxamer 188 described in A and B below, for dosage forms having T₉₀s of 6 or 8 hours, or 80% cellulose acetate 398-10 and 20% poloxamer 188, for dosage forms having T₉₀s of 10 hours, described in C below, both containing trace amounts of BHT, 0.0003%) were dissolved in a solvent that consisted of 99.5% acetone and 0.5% water (w/w) and the appropriate amount of acetone and water were transferred into the mixing vessel. While mixing, the vessel was heated to 25° C. to 28° C. and then the hot water supply was turned off. The appropriate amount of poloxamer 188, cellulose acetate 398-10 and BHT were charged into the mixing vessel containing the preheated acetone/water solution. The materials were mixed together in the vessel until all the solids were dissolved.

The subcoated bilayer cores (approximately 9 kg per lot) were placed into a Vector Hi-Coater. The coater was started and after the target exhaust temperature was attained, the coating solution was sprayed onto the rotating tablet bed. At regular intervals throughout the coating process, the weight gain was determined. When the desired wet weight gain was achieved, the coating process was stopped.

To obtain coated cores having a particular T₉₀ value, the appropriate coating solution was uniformly applied to the rotating tablet bed until the desired membrane weight gain was obtained, as described in A, B and C below. At regular intervals throughout the coating process, the weight gain was determined and sample membrane coated units were tested in the release rate assay as described in Example 4 to determine a T₉₀ for the coated units.

The membrane was coated onto the bilayer cores to a weight gain of 40 mg and yielded a dosage form having a T₉₀ of about 6 hours in the release rate assay (i.e., approximately 90% of the drug is released from the dosage form in 6 hours).

The membrane was coated onto the bilayer cores to a weight gain of 59 mg, yielding a dosage form having a T₉₀ of about 8 hours, as determined in the release rate assay.

The membrane was coated onto the bilayer cores to a weight gain of 60 mg and yielded a dosage form having a T₉₀ of about 10 hours in the release rate assay.

Drilling of Membrane Coated Systems

One exit port was drilled into the drug layer end of the membrane coated system.

During the drilling process, samples were checked at regular intervals for orifice size, location, and number of exit ports.

Drying of Drilled Coated Systems

Prior to drying, twinned and broken systems were removed from the batch as necessary. The tablets were manually passed through perforated trays to sort out and remove twinned systems. One exit port was drilled into the coated cores using the LCT laser. The exit port diameter was targeted at 4.5 mm, which was drilled on the drug layer dome of the membrane-coated cores. During the drilling process, three tablets were removed for orifice size measurement periodically. Acceptable Quality Limit (AQL) inspection was performed as well.

Drilled coated systems prepared as above were placed on perforated oven trays and placed on a rack in a relative humidity oven at 45° C. and 45% relative humidity and dried for 72 hours to remove residual solvent. Humidity drying was followed by at least 4 hours of drying at 45° C. and ambient relative humidity.

Application of the Drug Coating

A drug coating was provided over the drilled dosage forms described above. The coating included 6.6 wt % film-forming agent formed of a blend of HPMC 2910 (supplied by Dow) and copovidone (Kollidon® VA 64, supplied by BASF). The HPMC accounted for 3.95 wt % of the drug coating and the Kollidon® VA 64 accounted for 2.65 wt % of the drug coating. The drug coating also included HPC (Klucel® MF) as a viscosity enhancer. The HPC accounted for 1.0 wt % of the drug coating. APAP and HBH were included in the drug coating, with the two drugs accounting for 92.4 wt % of the drug coating. APAP accounted for 90 wt % of the drug coating, HBH accounted for 2.4 wt % of the drug coating.

In order to form the drug coating, an aqueous coating formulation was created using purified water USP as the solvent. The coating formulation included a solids content of 20 wt % and a solvent content of 80 wt %. The solids loaded into the coating formulation were those that formed the finished drug coating, and the solids were loaded in the coating formulation in the same relative proportions as contained in the finished drug coating. Two stainless steel vessels were used initially for mixing two separate polymer solutions, and then the polymer solutions were combined before adding HBH and APAP. Copovidone was dissolved in the first vessel, containing 24 kg of water (⅔ of the total water) followed by the addition of HPMC E-5. This vessel was equipped with two mixers, one of which was set up on the top and the other was located on the side at the bottom of the vessel. The Klucel MF (HPC) was dissolved in the second vessel containing 1200 grams of water (⅓ of the required water). Both polymer solutions were mixed until the solutions were clear. Next, the HPC/water solution was transferred into the vessel, which contained copovidone/HPMC/water. Then, HBH was added and mixed until dissolved completely. Finally, APAP (and optionally Ac-di-sol) was added to the polymer/HBH/water solution. The mixture was stirred continuously until a homogenous suspension was obtained. The suspension was mixed during spraying.

After forming the coating formulation, the drug coating was formed over the drilled dosage forms using a 24-inch High-Coater (CA# 66711-1-1) equipped with two Marsterflex peristattic pump heads. All of the three lots were coated to the same target weight gain of 195 mg/core (average coating weight of 199.7 mg).

Color and Clear Overcoats

Optional color or clear coats solutions were prepared in a covered stainless steel vessel. For the color coat, 88 parts of purified water was mixed with 12 parts of Opadry II until the solution was homogeneous. For the clear coat 90 parts of purified water was mixed with 10 parts of Opadry Clear until the solution was homogeneous. The dried cores prepared as above were placed into a rotating, perforated pan coating unit. The coater was started and after the coating temperature was attained (35-45° C.), the color coat solution was uniformly applied to the rotating tablet bed. When a sufficient amount of solution was applied, as conveniently determined when the desired color overcoat weight gain was achieved, the color coat process was stopped. Next, the clear coat solution was uniformly applied to the rotating tablet bed. When a sufficient amount of solution was applied, or the desired clear coat weight gain was achieved, the clear coat process was stopped. A flow agent (e.g., Carnubo wax) can be optionally applied to the tablet bed after clear coat application.

The components which make up the dosage forms described above are set forth as weight percent composition in Table 1 below.

TABLE 1
Formulations for Hydrocodone Bitartrate/Acetaminophen Tablets
Push Displacement Layer: 138 mg

| | |
|---|---|
| Polyethylene Oxide, NF, 303, 7000K, TG, LEO | 64.30 |
| Sodium Chloride, USP, Ph Eur, (Powder) | 30.00 |
| Povidone, USP, Ph Eur, (K29-32) | 5.00 |
| Ferric Oxide, NF, (Red) | 0.40 |
| Stearic Acid, NF, Powder | 0.25 |
| BHT, FCC, Ph Eur, (Milled) | 0.05 |

Drug Layer: 413 mg

| | |
|---|---|
| Polyethylene Oxide, NF, N-80, 200K, TG, LEO | 2.55 |
| Hydrocodone Bitartrate, USP | 2.42 |
| Acetaminophen, USP (fine powder) | 80.00 |
| Poloxamer F188 (Pluronic F68), NF, Ph Eur | 8.00 |
| Croscarmellose Sodium, NF | 3.00 |
| Povidone, USP, Ph Eur, (K29-32) | 3.00 |
| Stearic Acid, NF, Powder | 0.75 |
| Magnesium Stearate, NF, Ph Eur | 0.25 |
| BHT, FCC, Ph Eur, (Milled) | 0.03 |

Subcoating: 17 mg

| | |
|---|---|
| Hydroxyethyl Cellulose, NF | 95.0 |
| Polyethylene Glycol 3350, NF, LEO | 5.0 |

Membrane Coating*: 40 mg, 59 mg, 60 mg (for a
T₉₀ of 6 hrs, 8 hrs, and 10 hrs, respectively)

| | |
|---|---|
| Cellulose Acetate, NF, (398-10) | 75.0 (80.0) |
| Poloxamer F188 (Pluronic F68), NF, Ph Eur | 25.0 (20.0) |
| BHT, FCC, Ph Eur, (Milled) | Trace (0.0003) |

Drug Coating: 195 mg

| | |
|---|---|
| Hydrocodone Bitartrate, USP | 2.40 |
| Acetaminophen, USP (fine powder) | 90.00 |
| HPMC 2910, USP, Ph Eur, 5 cps | 3.96 |
| Copovidone, Ph Eur, JPE | 2.64 |
| Hydroxypropyl Cellulose, NF, MF | 1.00 |

Color Overcoat: 30 mg

| | |
|---|---|
| OPADRY, White (YS-2-7063) | 100.00 |

75/25 CA398-10/Pluronic F68 used for the 6 h and 8 hr systems
*80/20 CA398-10* 80/20 CA398-10/Pluronic F68 used for the 10 h system

Dosage forms manufactured as described above were tested in release rate assays as described in Example 2, and were tested in humans in a clinical trial described in Example 3 below.

### EXAMPLE 2

The release rate of drug from the dosage forms described above was determined in the following standardized assay. The method involves releasing systems into 900 ml acidified water (pH 3). Aliquots of sample release rate solutions were injected onto a chromatographic system to quantify the amount of drug released during specified test intervals. Drugs were resolved on a C₁₈ column and detected by UV absorption (254 nm for acetaminophen). Quantitation was performed by linear regression analysis of peak areas from a standard curve containing at least five standard points.

Samples were prepared with the use of a USP Type 7 Interval Release Apparatus. Each dosage form to be tested was weighed, then glued to a plastic rod having a sharpened end, and each rod was attached to a release rate dipper arm. Each release rate dipper arm was affixed to an up/down reciprocating shaker (USP Type 7 Interval Release Apparatus), operating at an amplitude of about 3 cm and 2 to 4 seconds per cycle. The rod ends with the attached systems were continually immersed in 50 ml calibrated test tubes containing 50 ml of acidified H₂O (acidified to pH 3.00.±.0.05 with phosphoric acid), equilibrated in a constant temperature water bath controlled at 37° C. ±0.5° C. At the end of each time interval of 90 minutes, the dosage forms were transferred to the next row of test tubes containing fresh acidified water. The process was repeated for the desired number of intervals until release was complete. Then the solution tubes containing released drug were removed and allowed to cool to room temperature. After cooling, each tube was filled to the 50 ml mark with acidified water, each of the solutions was mixed thoroughly, and then transferred to sample vials for analysis by high pressure liquid chromatography (HPLC). Standard solutions of drug were prepared in concentration increments encompassing the range of 5 micrograms to about 400 micrograms and analyzed by HPLC. A standard concentration curve was constructed using linear regression analysis. Samples of drug obtained from the release test were analyzed by HPLC and concentrations of drug were determined by linear regression analysis. The amount of drug released in each release interval was calculated.

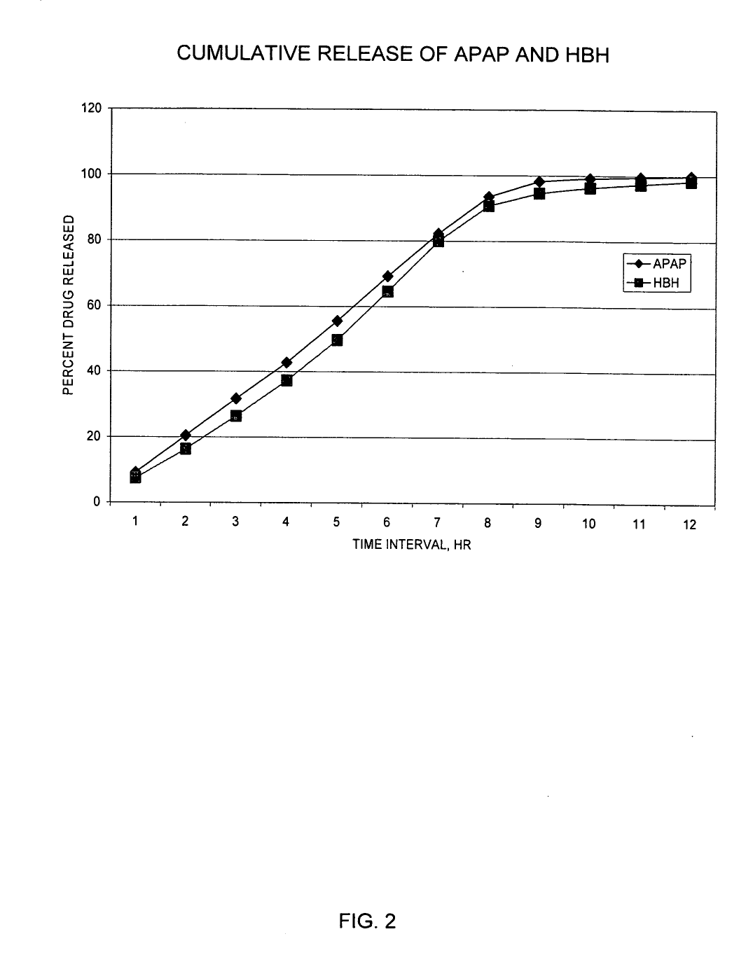

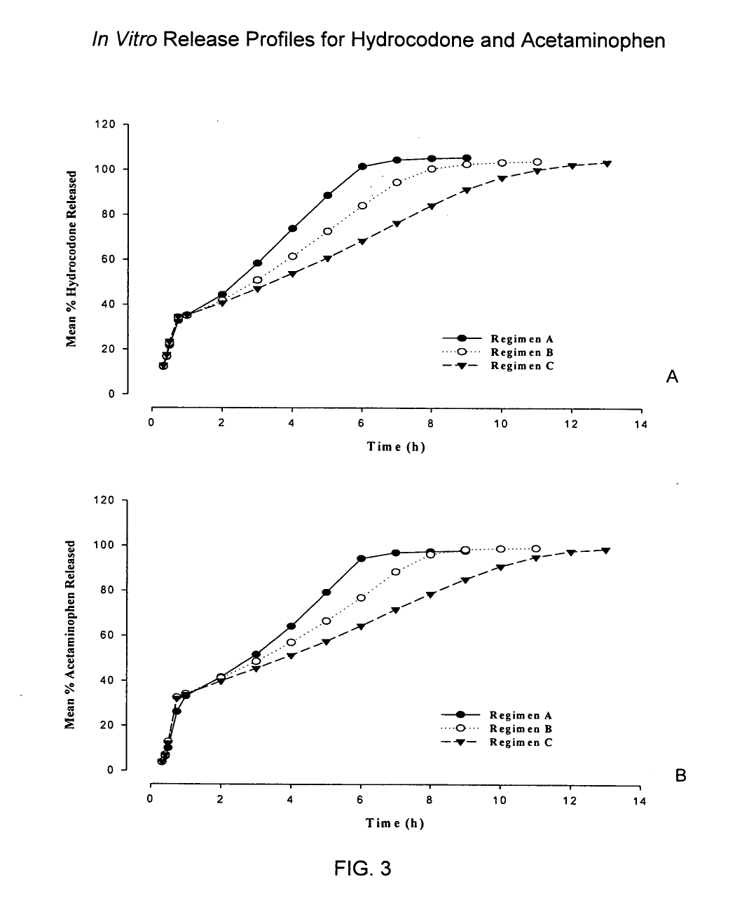

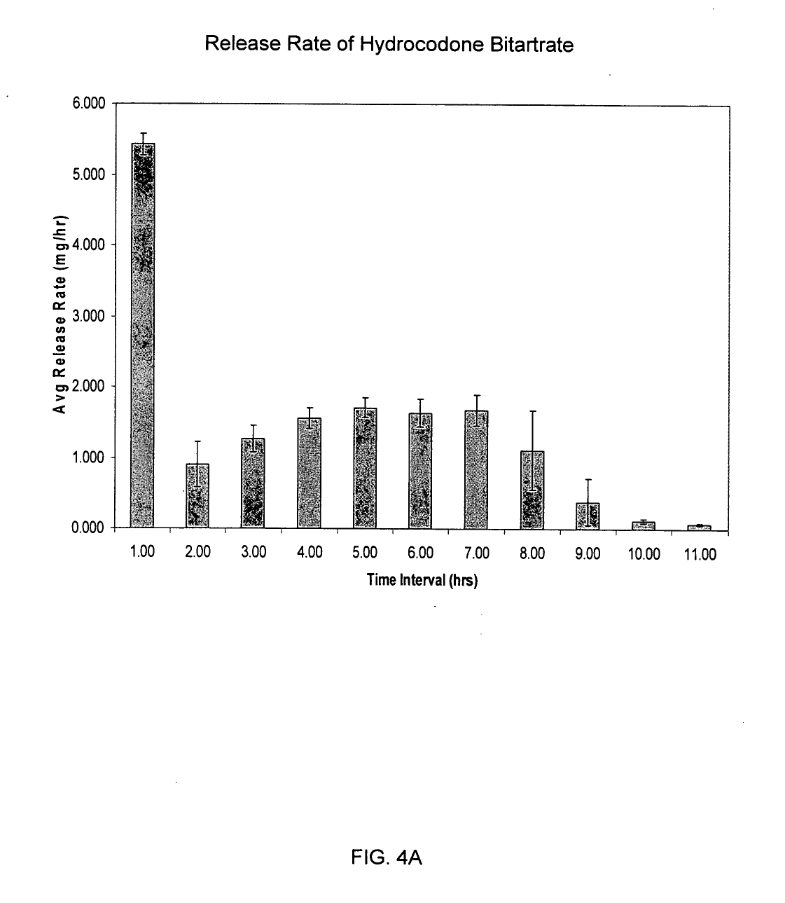

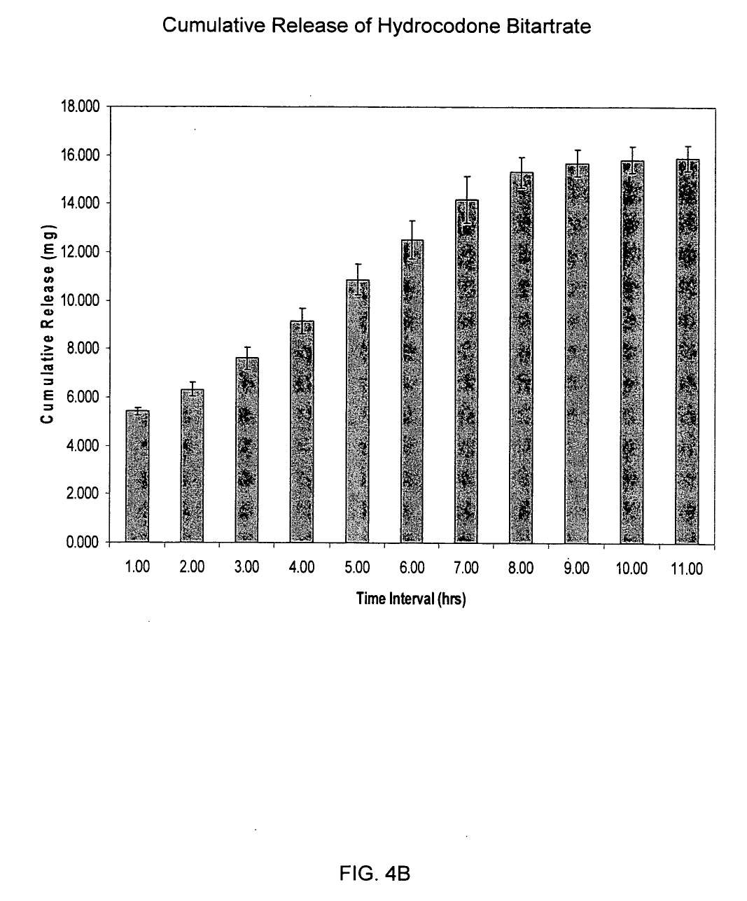

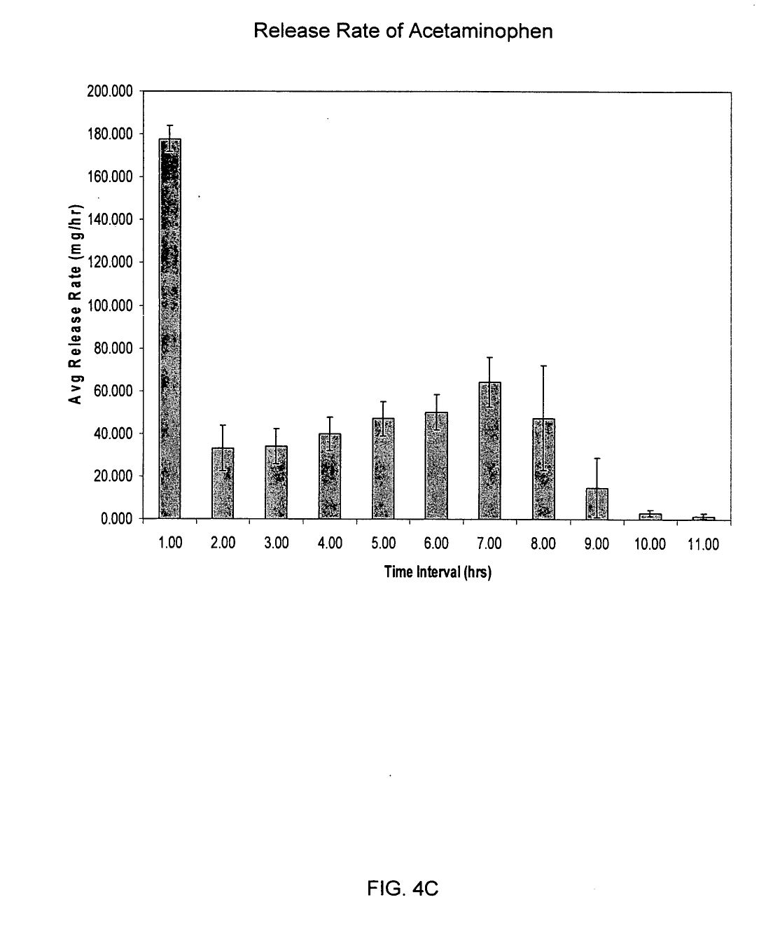

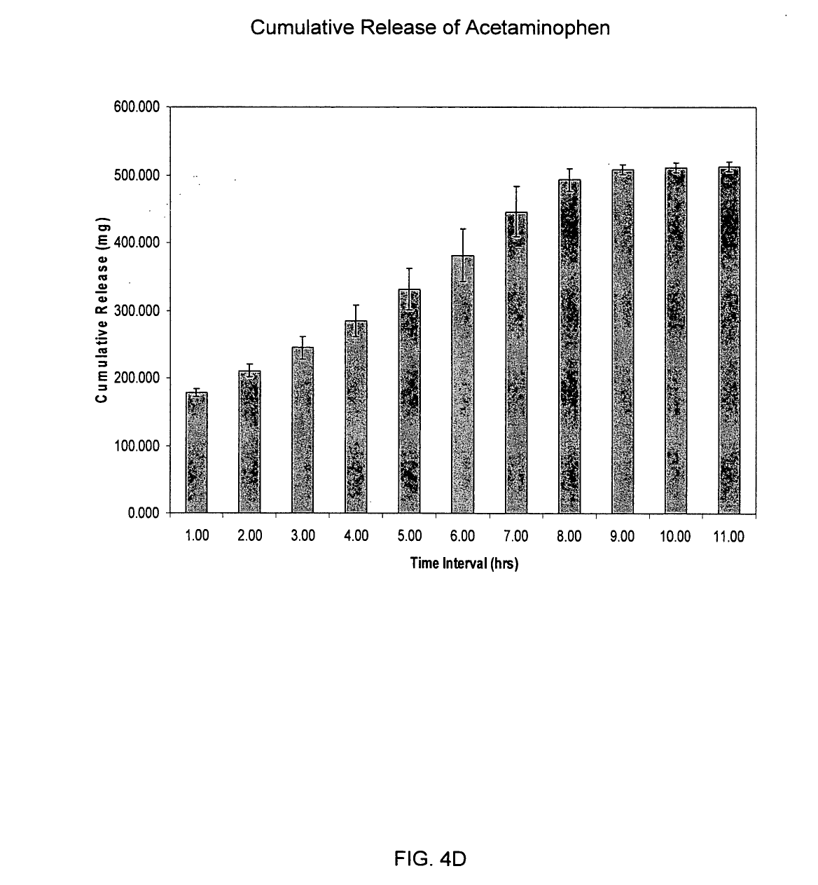

The release rate assay results for various dosage forms of the invention are illustrated in FIGS. 2-7, and in Table 2 below. Dosage forms having a membrane coating weight of 59 mg of 75/25 CA398-10/Pluronic F68 were shown to exhibit a T₉₀ of about 8 hours, as shown in FIGS. 2A and 2B, and the cumulative release rate graphs illustrated in FIG. 3 and FIGS. 5A-D. As can be seen from FIGS. 2 and 3, dosage forms release acetaminophen and hydrocodone at an ascending rate of release, whereby the percent drug released as a function of time does not exhibit a constant rate of release, but instead increases slightly with time until about 80% to 90% of the drug is released. The increase in the rate of release of acetaminophen and hydrocodone is due to the increased osmotic activity of the push displacement layer as the drug layer is expelled, and was observed in the absence as well as the presence of the drug coating. As shown in FIGS. 2A and 2B and FIG. 5A, dosage forms having a drug coating also exhibit an ascending rate of release, and exhibit an initial release of about ⅓ of the total dose from the drug coating. An initial peak hydrocodone release rate was observed occurring within one hour, and a second peak release rate was observed occurring within about 5 to 7 hours after introduction of the dosage form into the aqueous environment of the release assay. FIG. 5C also demonstrates the initial release of acetaminophen from the drug coating, followed by a slightly ascending rate of release until about 7 hours. The cumulative drug released is shown in FIG. 5B and 5D, for hydrocodone and acetaminophen, respectively, and demonstrates the initial drug release, followed by a slightly ascending rate of release.

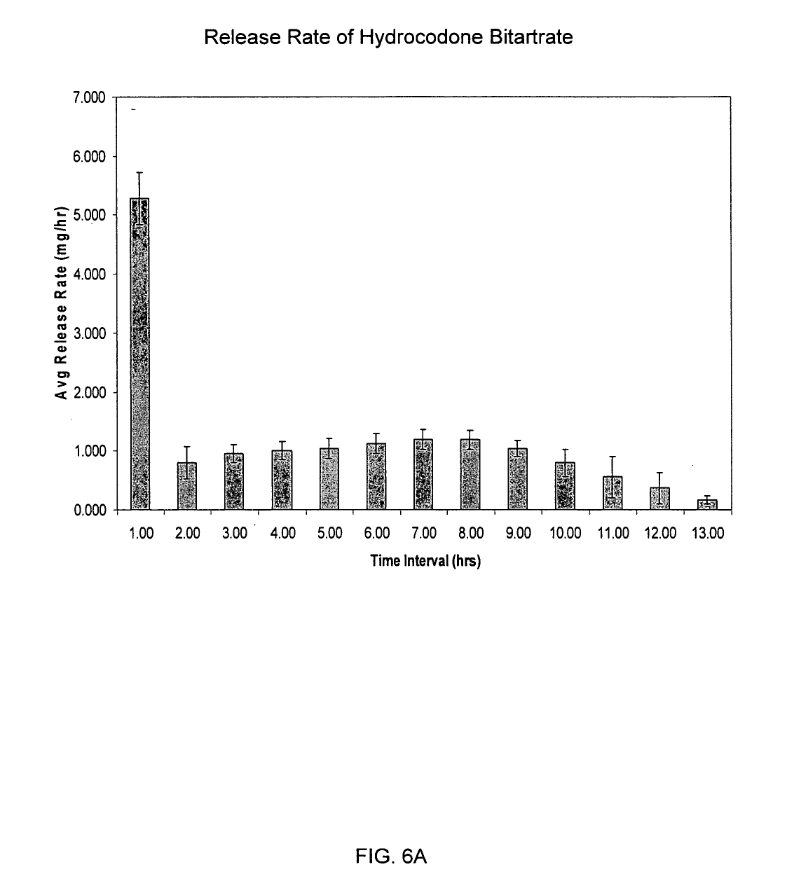

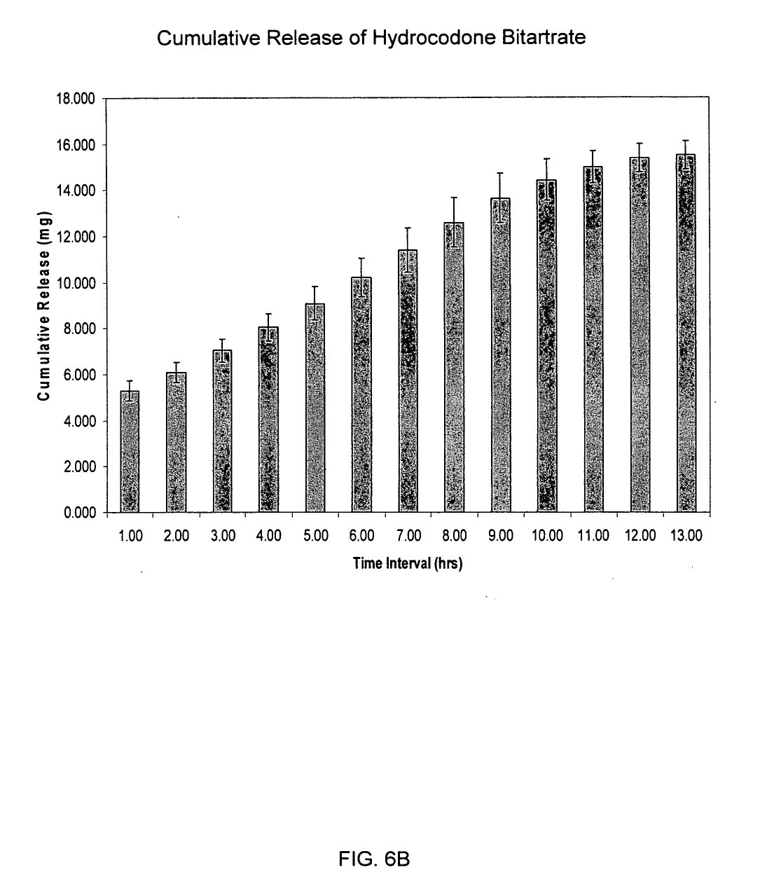

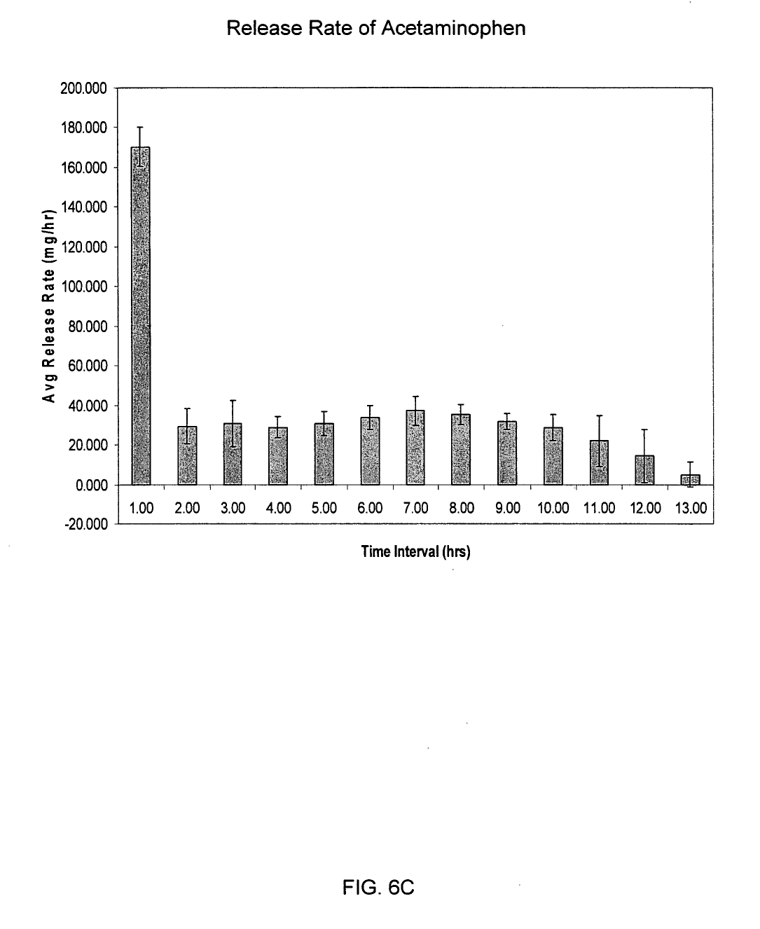

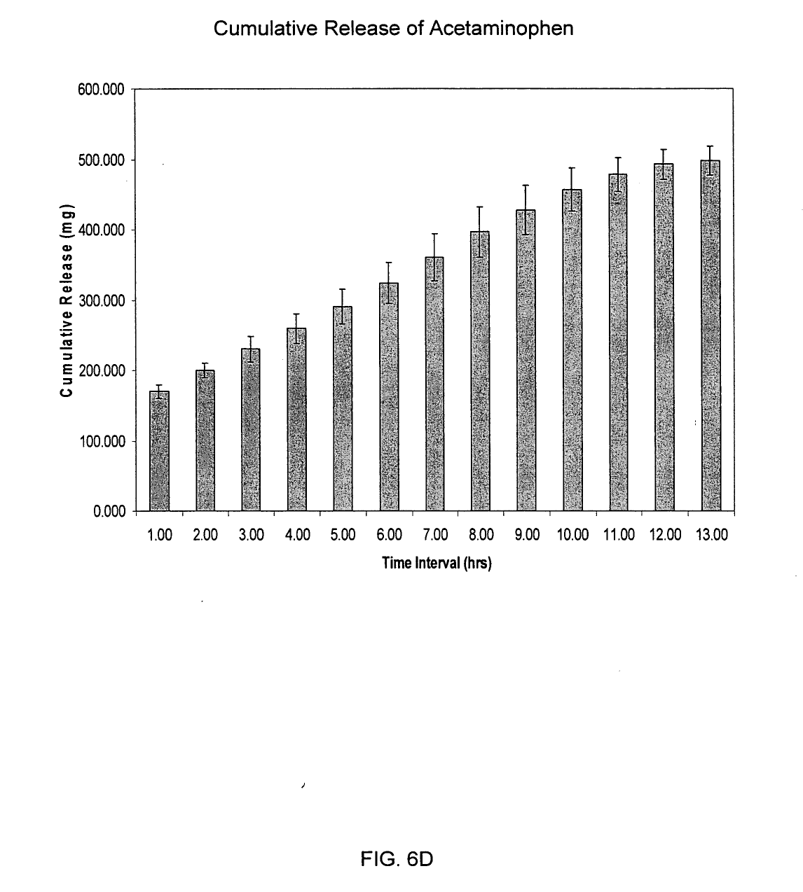

Dosage forms having a membrane coating weight of 40 mg of 75/25 CA398-10/Pluronic F68 were shown to exhibit a T₉₀ of about 6 hours, as shown in FIGS. 2A and 2B and FIGS. 6A-D. As shown in FIG. 6A, dosage forms having a drug coating exhibit an initial release of about ⅓ of the total dose of hydrocodone from the drug coating, followed by an ascending rate of release of hydrocodone to a second peak release rate occurring within about 4 to 6 hours. FIG. 6C demonstrates the initial release of acetaminophen from the drug coating, followed by a slightly ascending rate of release for about 5-6 hours. The cumulative drug released is shown in FIGS. 6B and 6D, for hydrocodone and acetaminophen, respectively, and demonstrates the initial drug release, followed by a slightly ascending rate of release.

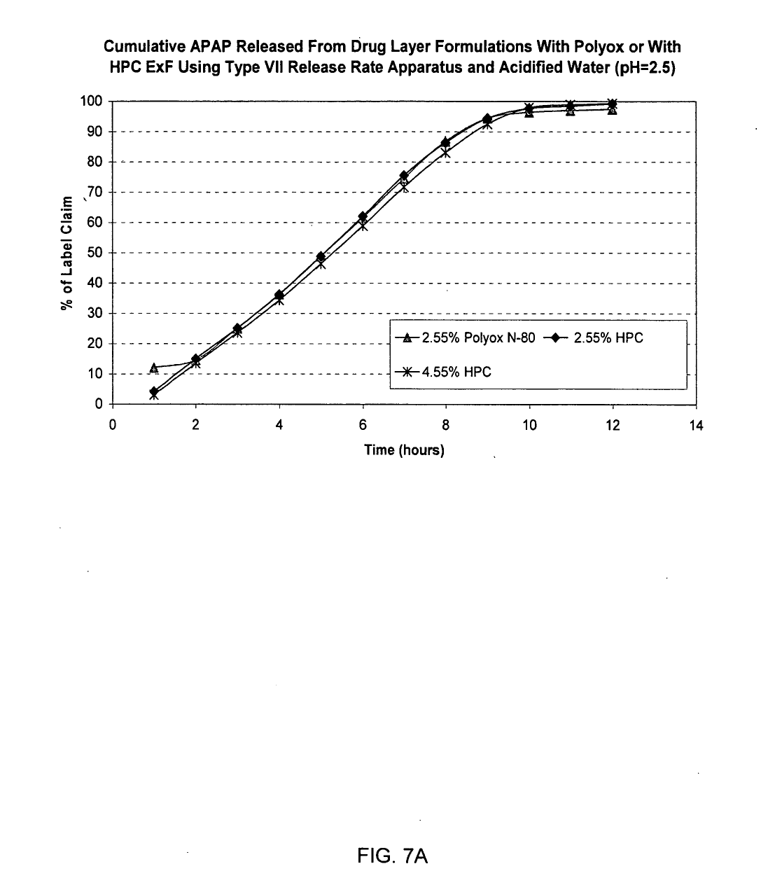

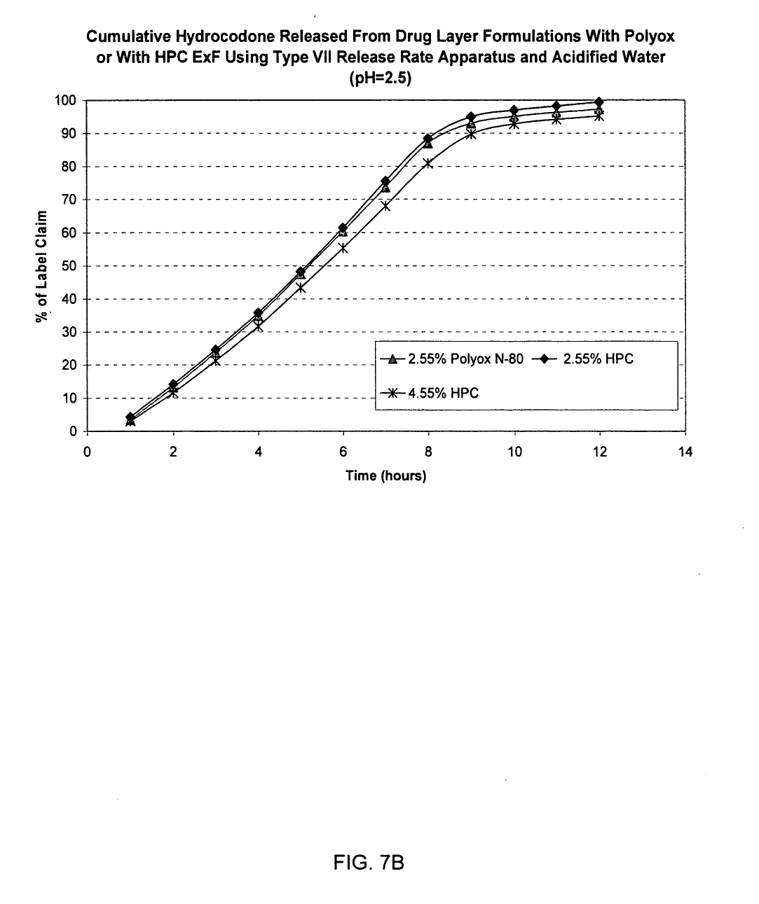

Dosage forms having a membrane coating weight of 60 mg of 80/20 CA398-10/Pluronic F68 were shown to exhibit a T₉₀ of about 10 hours, as shown in FIGS. 2A and 2B and FIGS. 7A-D. These dosage forms demonstrate a flatter release profile than the preceding systems characterized by having T₉₀ values of 6 and 8 hours. As shown in FIG. 7A, dosage forms having a drug coating exhibit an initial release of about ⅓ of the total dose of hydrocodone from the drug coating, followed by a slightly ascending rate of release of hydrocodone to a second peak release rate occurring within about 7 to 8 hours. FIG. 7C demonstrates the initial release of acetaminophen from the drug coating, followed by a slightly ascending rate of release for about 5-6 hours. The cumulative drug released is shown in FIGS. 7B and 7D, for hydrocodone and acetaminophen, respectively, and demonstrates the initial drug release, followed by a slightly ascending rate of release.

The results of the release rate assays performed on samples A, B and C from Example 1 are set forth in Table 2 below. Cumulative release is presented in Tables 3 and 4.

TABLE 2
Average release rate of acetaminophen and
hydrocodone bitartrate (mg/hr) vs. time

| | HBH | APAP | HBH | APAP | HBH | APAP |
|---|---|---|---|---|---|---|
| | T90 | T90 | T90 | T90 | T90 | T90 |
| Time | of 6 | of 6 | of 8 | of 8 | of 10 | of 10 |
| (hrs) | hours | hours | hours | hours | hours | hours |
| 1 | 5.144 | 164.378 | 5.428 | 177.803 | 5.286 | 170.256 |
| 2 | 1.373 | 42.583 | 0.899 | 32.983 | 0.801 | 29.392 |
| 3 | 2.193 | 51.915 | 1.275 | 34.049 | 0.953 | 30.723 |
| 4 | 2.327 | 62.988 | 1.564 | 39.841 | 1.008 | 28.873 |
| 5 | 2.115 | 74.093 | 1.715 | 47.010 | 1.033 | 30.787 |
| 6 | 1.891 | 73.522 | 1.641 | 49.893 | 1.120 | 33.903 |
| 7 | 0.338 | 11.099 | 1.678 | 64.175 | 1.199 | 37.039 |
| 8 | 0.109 | 2.930 | 1.115 | 47.327 | 1.190 | 35.382 |
| 9 | 0.064 | 1.374 | 0.383 | 14.841 | 1.042 | 31.560 |
| 10 | | | 0.118 | 2.782 | 0.792 | 28.561 |
| 11 | | | 0.073 | 1.589 | 0.558 | 21.930 |
| 12 | | | | | 0.370 | 14.434 |
| 13 | | | | | 0.168 | 5.084 |

As these data show, the dosage forms exhibit an ascending release rate over time. Due to the presence of the drug coating, the initial release rate from the sustained release dosage form cannot be determined at the 1 hour time point. However, the dosage forms show an increase in release rate from the 2 hour time point to a maximum occurring at about the T70 time interval, exhibiting increases of about 69% and 74% in release rate for hydrocodone bitartrate and acetaminophen, respectively, occurring between hours 2 and 5 for the dosage form having a T 90 of 6 hours; increases of about 86% and 96% in release rate for hydrocodone bitartrate and acetaminophen, respectively, occurring between hours 2 and 7 for the dosage form having a T 90 of 8 hours; and increases of about 48% and 20% in release rate for hydrocodone bitartrate and acetaminophen, respectively, occurring between hours 2 and 5 for the dosage form having a T 90 of 10 hours. The enhancement in release rate is most pronounced for dosage forms having T90s of less than 10 hours.

TABLE 3
Release pattern for acetaminophen (% released)

| | | | |
|---|---|---|---|
| | 6 hour | 8 hour | 10 hour |
| Time interval | formulation | formulation | formulation |

| | | | | |
|---|---|---|---|---|
| 0-20 | min | 4 | 4 | 4 |
| 0-25 | min | 6 | 7 | 7 |
| 0-30 | min | 10 | 13 | 12 |
| 0-45 | min | 26 | 34 | 32 |
| 0-1 | hour | 33 | 36 | 34 |
| 0-2 | hours | 42 | 42 | 40 |
| 0-3 | hours | 52 | 49 | 46 |
| 0-4 | hours | 64 | 57 | 51 |
| 0-5 | hours | 79 | 66 | 58 |
| 0-6 | hours | 94 | 76 | 64 |
| 0-7 | hours | 97 | 89 | 72 |
| 0-8 | hours | 98 | 99 | 79 |
| 0-9 | hours | 98 | 102 | 85 |
| 0-10 | hours | | 102 | 91 |
| 0-11 | hours | | 102 | 95 |
| 0-12 | hours | | | 98 |
| 0-13 | hours | | | 99 |

| | | | |
|---|---|---|---|
| residual | 0 | 1 | 1 |

TABLE 4
Release pattern for hydrocodone (% released)

| | | | |
|---|---|---|---|
| | 6 hour | 8 hour | 10 hour |
| Time interval | formulation | formulation | formulation |

| | | | | |
|---|---|---|---|---|
| 0-20 | min | 12 | 13 | 13 |
| 0-25 | min | 17 | 18 | 18 |
| 0-30 | min | 22 | 24 | 24 |
| 0-45 | min | 33 | 35 | 35 |
| 0-1 | hour | 35 | 36 | 35 |
| 0-2 | hours | 44 | 42 | 41 |
| 0-3 | hours | 58 | 51 | 47 |
| 0-4 | hours | 74 | 61 | 54 |
| 0-5 | hours | 89 | 73 | 61 |
| 0-6 | hours | 101 | 83 | 68 |
| 0-7 | hours | 104 | 95 | 76 |
| 0-8 | hours | 105 | 102 | 84 |
| 0-9 | hours | 105 | 105 | 91 |
| 0-10 | hours | | 105 | 97 |
| 0-11 | hours | | 106 | 100 |
| 0-12 | hours | | | 102 |
| 0-13 | hours | | | 103 |

| | | | |
|---|---|---|---|
| residual | 0 | 1 | 3 |

### EXAMPLE 3

The in vivo efficacy and safety of the dosage forms prepared in Example 1 were tested as follows:

Twenty-four healthy volunteers, twelve male and twelve female, were enrolled in a Phase I clinical trial of open label randomized four period crossover study design. An equal number of male subjects and female subjects were paired together in one of four groups. Subjects within each gender category were randomly assigned to the four sequences of regimens described below to avoid sequence bias and confounding of sequence and gender.

Four treatment options were tested in sequence, with a single treatment regimen administered on Study Day 1. A wash out period of at least 6 days was included to separate the dosing days. Each treatment group received each of the four treatments during the course of the study, as shown in Table 5 below with one exception. That exception was not included in the analysis of pharmacokinetic parameters. For the each of the four periods, subjects were given one of the four treatment options by oral administration, as follows:

a controlled release HBH/APAP product prepared by the method described in Example 1 (two tablets totaling 30 mg HBH and 1000 mg APAP), having a target T₉₀ value of approximately 6 hours (Regimen A);

a controlled release HBH/APAP product prepared by the method described in Example 1 (two tablets totaling 30 mg HBH and 1000 mg APAP), having a target T₉₀ value of approximately 8 hours (Regimen B);

a controlled release HBH/APAP product prepared by the method described in Example 1 (two tablets totaling 30 mg HBH and 1000 mg APAP), having a target T₉₀ value of approximately 10 hours (Regimen C); or

the reference drug NORCO®, an immediate release formulation of HBH and APAP containing 10 mg HBH and 325 mg APAP, administered every four hours for a total of three administrations over a 12 hour period (Regimen D).

TABLE 5
Regimen Sequence

| Sequence Group | Number of Subjects | Period 1 | Period 2 | Period 3 | Period 4 |
|---|---|---|---|---|---|
| I | M = 3, F = 3 | Regimen A | Regimen B | Regimen C | Regimen D |
| II | M = 3, F = 3 | Regimen B | Regimen D | Regimen A | Regimen C |
| III | M = 3, F = 3 | Regimen C | Regimen A | Regimen D | Regimen B |
| IV | M = 3, F = 3 | Regimen D | Regimen C | Regimen B | Regimen A |

The controlled release product of Regimens A-C and the first dose of Regimen D were administered on Study Day 1 under stringent fasting conditions. Blood samples were collected from each subject receiving treatment Regimens A-C for pharmacokinetic sampling at approximate times after administration as follows: 0, 0.25 hr, 0.5 hr, 0.75 hr, 1 hr, 2 hr, 3 hr, 4 hr, 6 hr, 8 hr, 10 hr, 12 hr, 16hr, 20 hr, 24 hr, 36 hr, 48 hr. For subjects receiving treatment Regimen D, blood samples were collected at approximate times after administration of the first dose as follows: 0, 0.25 hr, 0.5 hr, 0.75 hr, 1 hr, 2 hr, 4 hr, 4.25 hr, 4.5 hr, 5 hr, 6 hr, 8 hr, 8.25 hr, 8.5 hr, 9 hr, 10 hr, 12 hr, 16hr, 20 hr, 24 hr, 36 hr, 48 hr.

Blood samples were processed to separate plasma for further analysis, and plasma concentrations of hydrocodone and acetaminophen were determined using a validated HPLC/MS/MS method with quantitation between 0.092 and 92 ng/mL for hydrocodone and 5 and 10,000 ng/mL for acetaminophen.

Values for the pharmacokinetic parameters of hydrocodone and acetaminophen were estimated using noncompartmental methods. Plasma concentrations were adjusted for potency in the determination of pharmacokinetic parameters.

The maximum observed plasma concentration (Cₘₐₓ) and the time to Cₘₐₓ (peak time, Tₘₐₓ) were determined directly from the plasma concentration-time data. The value of the terminal phase elimination rate constant (β) was obtained from the slope of the least squares linear regression of the logarithms of the plasma concentration versus time data from the terminal log-linear phase of the profile. The terminal log-linear phase was identified using WinNonlin-Professional™, Version 4.0.1 (Pharsight Corporation, Mountain View, Calif.) and visual inspection. A minimum of three concentration-time data points was used to determine β. The terminal phase elimination half-life (t_{1/2}) was calculated as ln(2)/β.

The area under the plasma concentration-time curve (AUC) from time 0 to the time of the last measurable concentration (AUCₜ) was calculated by the linear trapezoidal rule. The AUC was extrapolated to infinite time by dividing the last measurable plasma concentration (Cₜ) by β. Denoting the extrapolated portion of the AUC by AUCₑₓₜ, the AUC from time 0 to infinite time (AUC_{∞}) was calculated as follows:
AUC_{∞}=AUCₜ+AUCₑₓₜ

The percentage of the contribution of the extrapolated AUC (AUCₑₓₜ) to the overall AUC_{∞} was calculated by dividing the AUCₑₓₜ by the AUC_{∞} and multiplying this quotient by 100. The apparent oral clearance value (CL/F, where F is the bioavailability) was calculated by dividing the administered dose by the AUC_{∞}.

Plasma concentrations of hydrocodone and acetaminophen along with their pharmacokinetic parameter values were tabulated for each subject and each regimen, and summary statistics were computed for each sampling time and each parameter.

The bioavailability of each CR regimen relative to that of the IR regimen was assessed by a two one-sided tests procedure via 90% confidence intervals obtained from the analyses of the natural logarithms of AUC. These confidence intervals were obtained by exponentiating the endpoints of confidence intervals for the difference of mean logarithms

The above analysis was performed on pharmacokinetic parameters adjusted for potency

Results

The plasma concentrations of hydrocodone and acetaminophen are shown in FIGS. 8A and 8B. As these figures illustrate, volunteers receiving two tablets of each of the three dosage forms prepared according the procedure of Example 1 exhibited a rapid rise in plasma concentrations of hydrocodone and acetaminophen after oral administration at time zero. The plasma concentrations of hydrocodone and acetaminophen reach an initial peak due to the release of hydrocodone and acetaminophen from the drug coating. Subsequent to the initial release of hydrocodone and acetaminophen, the sustained release of the dosage forms provides for continued release of hydrocodone and acetaminophen to the patient.

The test Regimens A (6 hour release prototype), B (8 hour release prototype) and C (10 hour release prototype) were equivalent to the reference Regimen D (NORCO®) with respect to AUC for both hydrocodone and acetaminophen because the 90% confidence intervals for evaluating bioequivalence were contained within the 0.80 to 1.25 range.

Test Regimen A was equivalent to the reference Regimen D with respect to hydrocodone Cₘₐₓ because the 90% confidence interval for evaluating bioequivalence was contained within the 0.80 to 1.25 range. Compared to Regimen D, hydrocodone Cₘₐₓ central values for Regimens B and C were 16% and 25% lower. Compared to Regimen D, acetaminophen Cₘₐₓ central values for Regimens A, B and C were 9% to 13% lower.

### EXAMPLE 4

Formulations were prepared to investigate the in vitro/in vivo correlation provided by certain formulations. The formulations were prepared as described in Example 1, using compositions as set forth in Table 6, with the exception that the drug coating and clear coats were omitted from these formulations. The semipermeable membrane composition was 75% cellulose acetate/25% poloxamer 188. Formulation #1 also contained 0.75% stearic acid and 0.25% magnesium stearate as a lubricant, while formulation #2 contained 1.0% stearic acid as a lubricant. In vitro release measurements were made as described above in Example 2.

TABLE 6
Composition of Formulations (wt %)

| | | | | | | |
|---|---|---|---|---|---|---|
| 1 | 85% | 2.58% | 5.39% | 3.0% | none | HPC 3.0% |
| 2 | 80% | 2.58% | 2.42% | 3.0% | Poloxmer 8.0% | PVP 3.0% |

The in vivo performance was tested in dogs by administering three dosage forms at 2 hour intervals for 12 hours. All the systems were retrieved after the 13^{th} hour and were analyzed for residual drug. Transit times and the robustness of the systems in vivo were also determined. The amounts of residual drug were correlated with the transit time.

Results of the in vivo studies demonstrated that dosage forms having no surfactant delivered active agent in vitro, but in vivo delivery was delayed in some cases due to adherence of undissolved drug layer onto the dosage form. It was concluded that for complete delivery of acetaminophen from the dosage forms and achieving a good in vitro/in vivo correlation, the presence of the higher amount of surfactant was desirable, at least with dosage forms containing the high concentrations of acetaminophen that were tested.

### EXAMPLE 5

Additional formulations were prepared to investigate alternative binding agents, disintegrant, polyox N-80, and surfactants to provide controlled release of dosage forms containing a high loading of acetaminophen and a smaller amount of hydrocodone bitartrate. These formulations were prepared according to the general procedures set forth in Example 1, using the following compositions, and the formulations lacked a drug coating or clear coat. The semipermeable membrane composition was 75% cellulose acetate/25% poloxamer 188. All formulations contained an additional 1% lubricant.

TABLE 7
Composition of drug layer in representative formulations (wt %)

| | | | | Croscarmellose | | |
|---|---|---|---|---|---|---|
| | | | | sodium (or | | |
| | | | Polyox | other | | |
| Formulation | APAP | HBH | N-80 | disintegrant) | Surfactant | Binder |
| 1 | 85% | 2.58% | 2.42% | 3.0% | Poloxamer | HPC 3.0% |
| | | | | | 3.0% | |
| 2 | 85% | 2.58% | 2.42% | 3.0% | Myrj, 3.0% | HPC 3.0% |
| 3 | 85% | 2.58% | 2.42% | 3.0% | Poloxamer | PVP 3.0% |
| | | | | | 3.0% | |
| 4 | 85% | 2.58% | 3.42% | 2.0% | Tween 80 | PVP 5.0% |
| | | | | | 1.0% | |
| 5 | 85% | 2.58% | 3.42% | 2.0% | Cremophor | PVP 5.0% |
| | | | | | EL 1.0% | |
| 6 | 85% | 2.58% | 1.42% | 2.0% | Poloxamer | PVP 5.0% |
| | | | | | 3.0% | |
| 7 | 85% | 2.58% | 2.42% | 3.0% | Poloxamer | HPC 3.0% |
| | | | | | 3.0% | |
| 8 | 85% | 2.58% | 1.42% | 3.0% | Tween 80 | HPC 3.0% |
| | | | | | 1.0% and | |
| | | | | | Poloxamer | |
| | | | | | 3.0% | |
| 9 | 85% | 2.58% | 2.42% | 3.0% | Myrj 52S | HPC 3.0% |
| | | | | | 3.0% | |
| 10 | 85% | 2.58% | 5.49% | Sodium starch | none | HPC 3.0% |
| | | | | glycolate 3.0% | | |
| 11 | 85% | 2.58% | 5.49% | Sodium | none | HPC 3.0% |
| | | | | alginate 3.0% | | |
| 12 | 80% | 2.42% | 2.55% | 3.0% | Poloxamer | PVP 3.0% |
| | | | | | 8.0% | |
| 13 | 78.79% | 2.38% | none | 3.0% | Poloxamer | PVP 3.0%, |
| | | | | | 8.0% | HPC 2.55% |
| 14 | 76.85% | 2.32% | none | 3.0% | Poloxamer | PVP 3.0%, |
| | | | | | 8.0% | HPC 4.55% |
| 15 | 80% | 2.42% | 2.55% | 3.0% | Poloxamer | PVP 3.0% |
| | | | | | 8.0% | |

These formulations were prepared and tested in an in vitro release rate assay as described in Example 2. The formulations generally released acetaminophen at a rate of about 20-60 mg/hr, and averaging approximately 40 mg/hr, for 8-9 hours. Formulations prepared using the surfactant Myrj had a comparable release rate and pattern of release to formulations prepared using Poloxamer.

Formulations 4-6 were prepared using micronized acetaminophen, and the release rate appeared to be more variable. The use of Tween 80 and Cremophor EL resulted in comparable release rates to Poloxamer or Myrj. Formulations 7-9 were prepared using non-micronized acetaminophen, and exhibited a more consistent release rate. Formulation #8 exhibited an initial burst release of about 80 mg/hr not seen with the additional formulations.

Formulations 10 and 11 were prepared using the alternative disintegrating agents sodium starch glycolate and sodium alginate. These two formulations were prepared without surfactant and exhibited more pronounced ascending rates of release.

Formulations 12-15 were prepared and tested as described. There was also 0.5% colloidal silicon dioxide in formulations 13 and 14, and there were slight variations in the amounts of the lubricants stearic acid and magnesium stearate in each of these formulations. The semipermeable membrane coating was 64 mg on each of these formulations, using a ratio of 77% cellulose acetate 398-10 and 23% poloxamer 188. The cumulative release rate of acetaminophen and hydrocodone from formulations #12-14 is shown in FIGS. 7A and B.

### EXAMPLE 6

A dosage form containing 350 mg ibuprofen was prepared using the procedures generally described in Example 1. The drug layer composition consisted of the following components: 80.86 wt % ibuprofen (USP, 25 micron), 4.5 wt % povidone, USP, Ph Eur (K29-32), 4.5 wt % HPC, JF, 4.0 wt % croscarmellose sodium, NF, 3.0 wt % sodium lauryl sulfate, NF, 1.74 wt % hydrocodone bitartrate, 1.0 wt % stearic acid, NF, 0.4 wt % magnesium stearate, NF. The push layer contained the following components: 63.67 wt % polyethylene oxide (7000K, NF), 30.0 wt % NaCl, 5 wt % povidone USP, Ph Eur (K29-32), 1 wt % magnesium stearate, NF, Ph Eur, JP, 0.25 wt % ferric oxide, NF, 0.08 wt % BHT, NF. The semipermeable membrane was composed of 75 wt % cellulose acetate, NF (398-10) and 25 wt % poloxamer 188, NF.

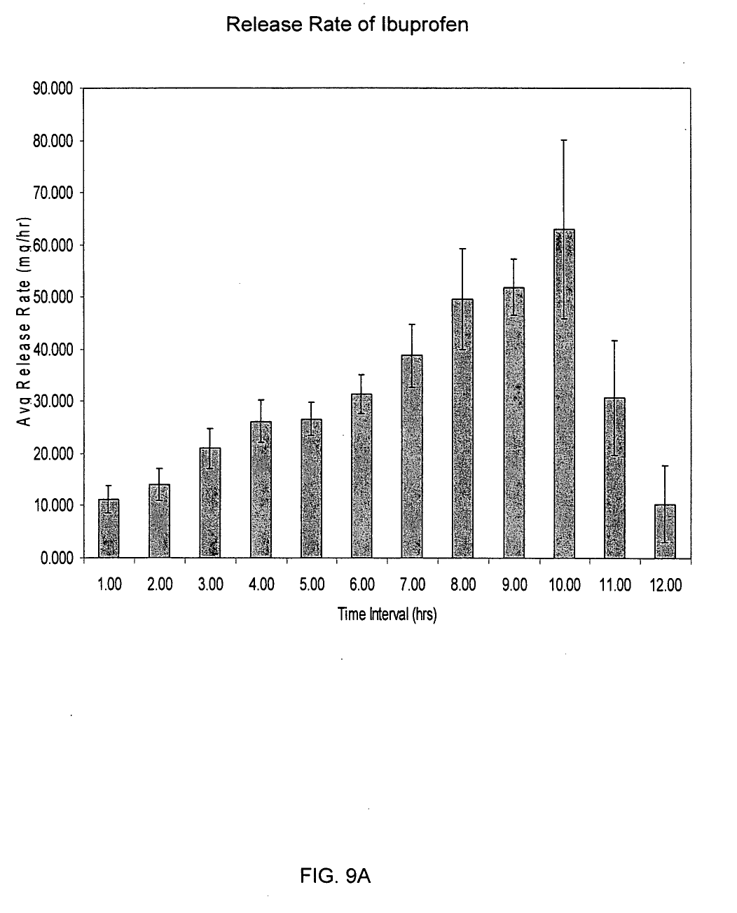

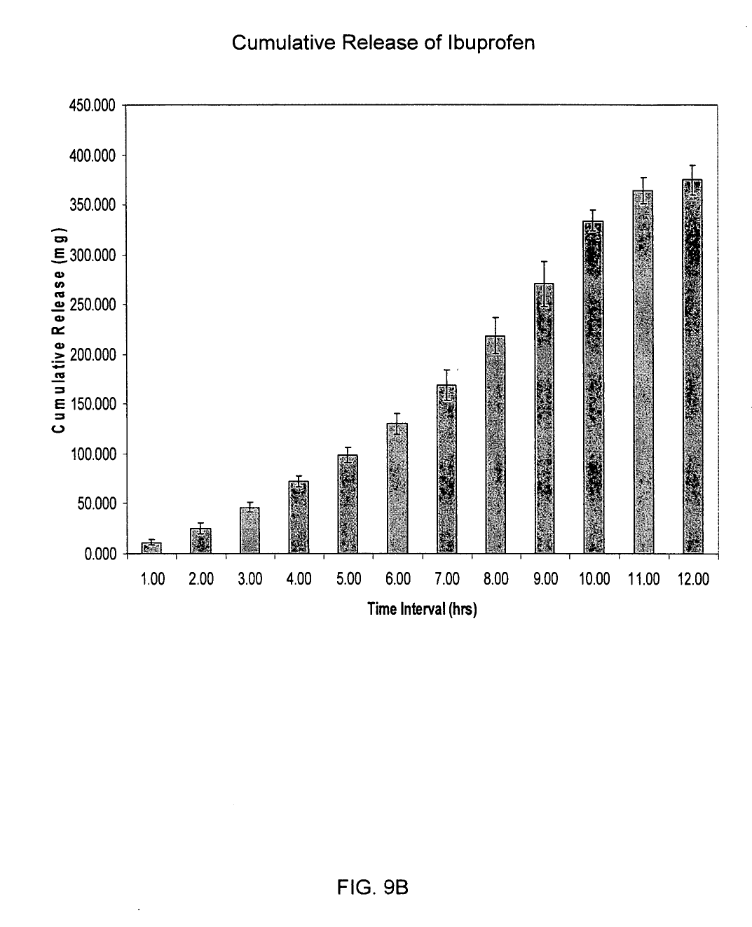

This dosage form produced an initial average rate of release of ibuprofen of 14.5 mg/hr for the first hour, followed by an ascending release rate up to a maximum release rate of about 50 mg/hr at 9 hours, and a sustained release overall for about 9 hours, before rapidly dropping off to baseline levels, with a T₉₀ of about 9 hours. The majority of the dose was delivered at an ascending release rate. The results are shown graphically in FIG. 9, with the release rate data shown in FIG. 9A and the cumulative release in FIG. 9B. These data demonstrate the absence of a burst release and the predominant ascending release delivery profile provided by this formulation containing povidone and no osmagent.

### EXAMPLE 7

A dosage form containing 350 mg ibuprofen was prepared using the procedures generally described in Example 1. The drug layer composition consisted of the following components: 81.85 wt % ibuprofen (USP, 25 micron), 8.0 wt % HPC, NF, 3.0 wt % povidone, USP, Ph Eur (K29-32), 4.0 wt % croscarmellose sodium, NF, 3.0 wt % sodium lauryl sulfate, NF, 1.75 wt % hydrocodone bitartrate, 1.0 wt % stearic acid, NF, 0.4 wt % magnesium stearate, NF. The push layer contained the following components: 63.67 wt % polyethylene oxide (7000K, NF), 30.0 wt % NaCl, 5 wt % povidone USP, Ph Eur (K29-32), 1 wt % magnesium stearate, NF, Ph Eur, JP, 0.25 wt % ferric oxide, NF, 0.08 wt % BHT, NF. The semipermeable membrane was composed of 75 wt % cellulose acetate, NF (398-10) and 25 wt % poloxamer 188, NF.

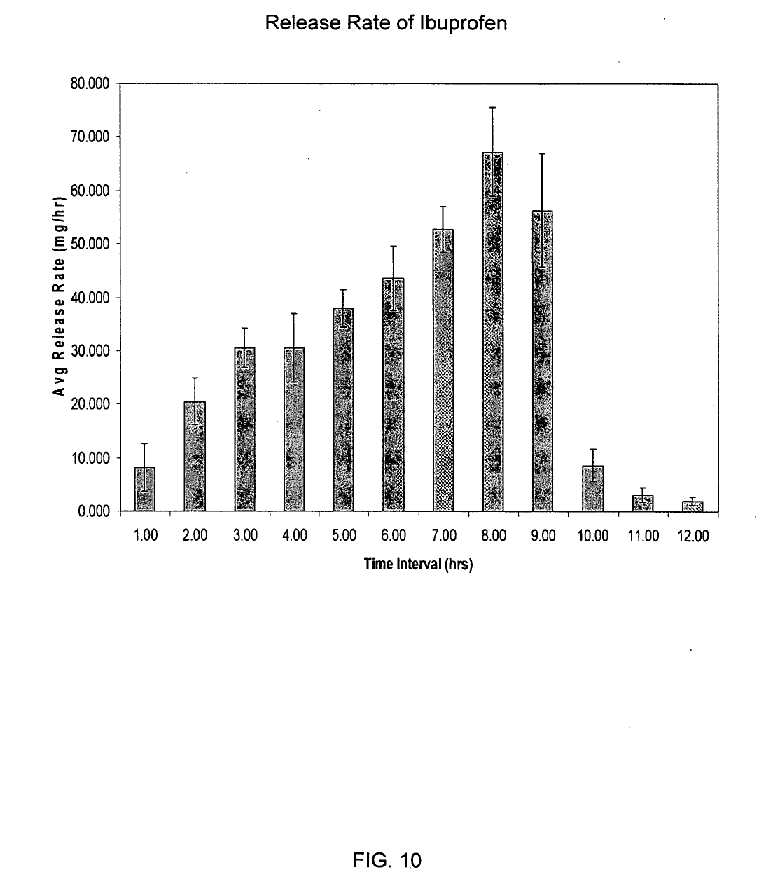

This dosage form produced an initial average rate of release of ibuprofen of 8.2 mg/hr for the first hour, followed by an ascending release rate up to a maximum release rate of about 67 mg/hr at 8 hours, and a sustained release overall for about 9 hours, before rapidly dropping off to baseline levels, with a T₉₀ of about 9 hours. The majority of the dose was delivered at an ascending release rate. The results are shown graphically in FIG. 10. These data demonstrate the absence of a burst release and the predominant ascending release delivery profile provided by this formulation containing a larger proportion of hydroxypropylcellulose and povidone and no osmagent.

The above-described exemplary embodiments are intended to be illustrative in all respects, rather than restrictive, of the present invention. Thus, the present invention is capable of implementation in many variations and modifications that can be derived from the description herein by a person skilled in the art. All such variations and modifications are considered to be within the scope and spirit of the present invention as defined by the following claims.

What is claimed is:

1. A sustained release dosage form comprising a pharmaceutically active agent and pharmaceutically acceptable salts thereof and adapted to release as an erodible solid over a prolonged period of time, wherein the dosage form provides an ascending rate of release of the pharmaceutically active agent for at least about 4 hours.

2. The sustained release dosage form of claim 1, wherein the dosage form provides an ascending rate of release of the pharmaceutically active agent for from about 5 to about 8 hours.

3. The sustained release dosage form of claim 1, wherein the dosage form provides an ascending rate of release of the pharmaceutically active agent until about 70% of the active agent has been released.

4. The sustained release dosage form of claim 1, wherein after the ascending rate of release, there is a rapid decrease in release rate.

5. The sustained release dosage form of claim 1, wherein the dosage form releases at least 90% of the active agent within 12 hours.

3. The sustained release dosage form of claim 1, wherein the erodible solid further comprises a surfactant.

4. The sustained release dosage form of claim 1, wherein the pharmaceutically active agent has a solubility of less than about 50 mg/ml at 25° C.

5. The sustained release dosage form of claim 4, wherein the pharmaceutically active agent has a solubility of less than about 10 mg/ml at 25° C.

6. The sustained release dosage form of claim 3, wherein the surfactant is a nonionic or ionic surfactant.

7. The sustained release dosage form of claim 6, wherein the nonionic surfactant is a poloxamer, polyoxyethylene ester, sugar ester surfactant, sorbitan fatty acid ester, glycerol fatty acid ester, polyoxyethylene ether of high molecular weight aliphatic alcohols, polyoxyethylene 40 sorbitol lanolin derivative, polyoxyethylene 75 sorbitol lanolin derivative, polyoxyethylene 20 sorbitol lanolin derivative, polyoxyethylene 50 sorbitol lanolin derivative, polyoxyethylene 6 sorbitol beeswax derivative, polyoxyethylene 20 sorbitol beeswax derivative, polyoxyethylene derivative of fatty acid esters of sorbitan, and mixtures thereof.

8. The sustained release dosage form of claim 7, wherein the nonionic surfactant is a poloxamer, a fatty acid ester of polyoxyethylene, a sugar ester surfactant, or mixtures thereof.

9. The sustained release dosage form of claim 1, wherein the erodible solid comprises from about 5 to about 15 percent by weight of a binding agent and a disintegrant.

10. The method of claim 1, wherein the erodible solid comprises from about 1 to about 15 percent by weight of a surfactant.

11. The sustained release dosage form of claim 1, wherein the pharmaceutically active agent is present in the erodible solid at a percent composition of at least about 20 weight percent.

12. The sustained release dosage form of claim 11, wherein the pharmaceutically active agent is present in the erodible solid at a percent composition of at least about 60 weight percent.

13. The sustained release dosage form of claim 12, wherein the pharmaceutically active agent is present in the erodible solid at a percent composition of from about 60 percent to about 95 percent by weight.

14. The sustained release dosage form of claim 11, wherein the pharmaceutically active agent is present in the erodible solid at a percent composition of from about 20 percent to about 95 percent by weight.

15. The sustained release dosage form of claim 14, wherein the pharmaceutically active agent is present in the erodible solid at a percent composition of from about 40 percent to about 95 percent by weight.

16. The sustained release dosage form of claim 15, wherein the pharmaceutically active agent is present in the erodible solid at a percent composition of from about 60 percent to about 95 percent by weight.

17. The sustained release dosage form of claim 16, wherein the pharmaceutically active agent is present in the erodible solid at a percent composition of from about 70 percent to about 90 percent by weight.

18. The sustained release dosage form of claim 17, wherein the pharmaceutically active agent is present in the erodible solid at a percent composition of from about 75 percent to about 85 percent by weight.

19. The sustained release dosage form of claim 1, further comprising at least one additional pharmaceutically active agent in the erodible solid.

20. The sustained release dosage form of claim 19, wherein the pharmaceutically active agents have similar solubilities.

21. The sustained release dosage form of claim 19, wherein the pharmaceutically active agents have different solubilities.

22. The sustained release dosage form of claim 19, wherein the pharmaceutically active agents are released from the dosage form at rates that are proportional relative to each other.

23. The sustained release dosage form of claim 1, further comprising an immediate release drug coating comprising an effective dose of at least one pharmaceutically active agent.

24. A sustained release dosage form for oral administration of a pharmaceutically active agent, comprising
(1) a semipermeable wall defining a cavity and including an exit orifice formed or formable therein;
(2) a drug layer comprising a therapeutically effective amount of a pharmaceutically active agent and pharmaceutically acceptable salts thereof contained within the cavity and located adjacent to the exit orifice;
(3) a push displacement layer contained within the cavity and located distal from the exit orifice;
(4) a flow-promoting layer in between the inner surface of the semipermeable wall and at least the external surface of the drug layer that is opposite the wall;
wherein the dosage form provides an ascending rate of release of the pharmaceutically active agent for at least about 4 hours.

25. The sustained release dosage form of claim 24, wherein the dosage form provides an ascending rate of release of the pharmaceutically active agent until about 70 percent of the active agent has been released.

26. The sustained release dosage form of claim 24, wherein the maximum rate of release exhibited by the dosage form is at least 20% greater than the minimum release rate exhibited by the dosage form.

27. The sustained release dosage form of claim 24, wherein the drug layer further comprises a binding agent, a disintegrant or mixtures thereof.

28. The sustained release dosage form of claim 24, wherein the drug layer further comprises a surfactant.

29. The sustained release dosage form of claim 28, wherein the surfactant is a nonionic or ionic surfactant.

30. The sustained release dosage form of claim 29, wherein the nonionic surfactant is a poloxamer, polyoxyethylene ester, sugar ester surfactant, sorbitan fatty acid ester, glycerol fatty acid ester, polyoxyethylene ether of high molecular weight aliphatic alcohols, polyoxyethylene 40 sorbitol lanolin derivative, polyoxyethylene 75 sorbitol lanolin derivative, polyoxyethylene 20 sorbitol lanolin derivative, polyoxyethylene 50 sorbitol lanolin derivative, polyoxyethylene 6 sorbitol beeswax derivative, polyoxyethylene 20 sorbitol beeswax derivative, polyoxyethylene derivative of fatty acid esters of sorbitan, and mixtures thereof.

31. The sustained release dosage form of claim 30, wherein the nonionic surfactant is a poloxamer, a fatty acid ester of polyoxyethylene, a sugar ester surfactant, or mixtures thereof.

32. The sustained release dosage form of claim 24, wherein the pharmaceutically active agent is present in the drug layer at a percent composition of at least about 20 weight percent.

33. The sustained release dosage form of claim 32, wherein the pharmaceutically active agent is present in the drug layer at a percent composition of from about 20 percent to about 95 percent by weight.

34. The sustained release dosage form of claim 33, wherein the pharmaceutically active agent is present in the drug layer at a percent composition of from about 40 percent to about 95 percent by weight.

35. The sustained release dosage form of claim 34, wherein the pharmaceutically active agent is present in the drug layer at a percent composition of from about 60 percent to about 95 percent by weight.

36. The sustained release dosage form of claim 35, wherein the pharmaceutically active agent is present in the drug layer at a percent composition of from about 70 percent to about 90 percent by weight

37. The sustained release dosage form of claim 24, wherein the pharmaceutically active agent has a solubility of less than about 50 mg/ml at 25° C.

38. The sustained release dosage form of claim 24, wherein the pharmaceutically active agent has a solubility of less than about 10 mg/ml at 25° C.

39. The sustained release dosage form of claim 24, wherein the drug layer further comprises at least one additional pharmaceutically active agent.

40. The sustained release dosage form of claim 39, wherein the pharmaceutically active agents have similar solubilities.

41. The sustained release dosage form of claim 39, wherein the pharmaceutically active agents have different solubilities.

42. The sustained release dosage form of claim 39, wherein the pharmaceutically active agents are released from the dosage form at rates that are proportional relative to each other.

43. The sustained release dosage form of claim 24, wherein the drug layer is exposed to the environment of use as an erodible composition.

44. The sustained release dosage form of claim 24, further comprising an immediate release drug coating comprising an effective dose of at least one pharmaceutically active agent.

45. The sustained release dosage form of claim 24, wherein the pharmaceutically active agent is selected from a nonopioid analgesic agent, an antibiotic, an antiepileptic agent, or combinations thereof.

46. The sustained release dosage form of claim 39, wherein the at least one additional pharmaceutically active agent is selected from an opioid analgesic agent, a gastric protective agent, or a 5-HT agonist.

47. The sustained release dosage form of claim 24, further comprising a drug coating comprising a therapeutically effective amount of the pharmaceutically active agent sufficient to provide an immediate effect in a patient in need thereof.

48. A method for providing a sustained release of an increasing dose of a pharmaceutically active agent to a patient in need thereof, comprising orally administering a dosage form comprising a pharmaceutically active agent and pharmaceutically acceptable salts thereof, a binding agent, and a disintegrant adapted to release as an erodible solid over a prolonged period of time, wherein the dosage form provides an ascending rate of release of the pharmaceutically active agent for at least about 4 hours.

49. A method for providing a sustained release of a therapeutically effective dose of a pharmaceutically active agent characterized by administration to a patient in a high dosage, low solubility and/or poor dissolution rate, comprising orally administering a dosage form comprising a pharmaceutically active agent and pharmaceutically acceptable salts thereof adapted to release as an erodible solid over a prolonged period of time, wherein the erodible solid comprises at least 60% by weight of the pharmaceutically active agent, and wherein said dosage form provides an ascending rate of release of the pharmaceutically active agent for at least about 4 hours.

50. A method for providing a therapeutically effective dose of a pharmaceutically active agent to a patient in need thereof, comprising orally administering a composition comprising a therapeutically effective amount of a pharmaceutically active agent present in at least 20% by weight in a drug layer contained within a cavity defined by an at least partially semipermeable wall and having an exit means located adjacent thereto, a push displacement layer located within the cavity distal from the exit means providing a sustained release of the composition from the cavity when placed in an aqueous environment of use, and a flow-promoting layer located in between the inner surface of the semipermeable wall and at least the external surface of the drug layer that is opposite the wall, wherein the drug layer is exposed to the environment of use as an erodible solid, and wherein the dosage form provides an ascending rate of release of the pharmaceutically active agent for at least about 4 hours.

51. The method of claim 50, further comprising a drug coating comprising a therapeutically effective amount of an immediate release therapeutic composition located on the outside surface of the at least partially semipermeable wall.

52. The method of claim 50, wherein the therapeutic composition provides an ascending rate of release of the pharmaceutically active agent for from about 5 hours to about 8 hours.

53. The method of claim 50, wherein the erodible solid comprises from about 20 to about 95% of the pharmaceutically active agent by weight.

54. The method of claim 53, wherein the erodible solid comprises from about 40 to about 95% of the pharmaceutically active agent by weight.

55. The method of claim 54, wherein the erodible solid comprises from about 60 to about 95% of the pharmaceutically active agent by weight.

56. The method of claim 55, wherein the erodible solid comprises from about 75 to about 85% of the pharmaceutically active agent by weight.

57. The method of claim 50, wherein the erodible solid comprises from about 5 to about 15 percent by weight of a binding agent and a disintegrant.

58. The method of claim 50, wherein the erodible solid comprises from about 1 to about 15 percent by weight of a surfactant.

59. A method for providing an effective concentration in the plasma of a patient of a pharmaceutically active agent that is metabolized relatively rapidly, comprising orally administering a therapeutic composition comprising a pharmaceutically active agent and pharmaceutically acceptable salts thereof adapted to release as an erodible solid over a prolonged period of time, wherein the erodible solid comprises the pharmaceutically active agent, and wherein said therapeutic composition provides an ascending rate of release of the pharmaceutically active agent for at least about 4 hours.

60. The method of claim 59, wherein the therapeutic composition further comprises a drug coating comprising a therapeutically effective amount of the pharmaceutically active agent sufficient to provide an immediate effect in a patient in need thereof.

61. The method of claim 59, wherein the therapeutic composition provides an ascending rate of release of the pharmaceutically active agent for from about 4 hours to about 8 hours.

62. The method of claim 59, wherein therapeutic composition provides a substantially zero order plasma profile of the pharmaceutically active agent in the patient.

63. The method of claim 59, wherein therapeutic composition provides an ascending plasma profile of the pharmaceutically active agent in the patient.

64. The method of claim 59, wherein therapeutic composition provides a declining plasma profile of the pharmaceutically active agent in the patient.

65. The method of claim 60, wherein the immediate release drug coating provides a therapeutically effective amount of the pharmaceutically active agent in the plasma of the patient and the ascending rate of release provided by the therapeutic composition maintains the concentration of the pharmaceutically active agent in the therapeutic range in the plasma of the patient for a prolonged period of time.

66. The method of claim 59, wherein the erodible solid comprises from about 20 to about 95% of the pharmaceutically active agent by weight.

67. The method of claim 66, wherein the erodible solid comprises from about 60 to about 95% of the pharmaceutically active agent by weight.

68. The method of claim 59, wherein the erodible solid comprises from about 5 to about 15 percent by weight of a binding agent and a disintegrant.

69. The method of claim 59, wherein the erodible solid comprises from about 1 to about 15 percent by weight of a surfactant.

70. A method for providing an effective dose of a pharmaceutically active agent to which tolerance develops relatively rapidly in a patient, comprising orally administering a therapeutic composition comprising an effective dose of a pharmaceutically active agent to which tolerance develops relatively rapidly contained in a drug layer, an osmotic push composition, an at least partially semipermeable wall, and an exit means in the wall for delivering the therapeutic composition from the dosage form, and a flow-promoting layer located in between the inner surface of the semipermeable wall and at least the external surface of the drug layer that is opposite the wall, wherein said drug layer and push composition are surrounded by the at least partially semipermeable wall, wherein the drug layer is exposed to the environment of use as an erodible composition, and further wherein said dosage form provides an ascending rate of release of the pharmaceutically active agent thereby providing increasing concentrations of the pharmaceutically active agent in the plasma of the patient.

71. A method for treating pain in a human patient in need thereof, comprising orally administering a dosage form comprising a therapeutic composition comprising a nonopioid analgesic, an opioid analgesic and pharmaceutically acceptable salts thereof adapted to release as an erodible solid over a prolonged period of time, wherein the nonopioid analgesic and the opioid analgesic are released at rates proportional relative to each other, and wherein said therapeutic composition provides an ascending rate of release of the nonopioid analgesic and the opioid analgesic for at least about 4 hours.

72. The method of claim 71, wherein the nonopioid analgesic is present in a weight percent of about 60% to about 95% of the erodible solid by weight.

73. The method of claim 71, wherein the nonopioid analgesic is present in a weight percent of about 70% to about 90% of the erodible solid by weight.

74. The sustained release dosage form of claim 19, wherein the pharmaceutically active agents are released from the dosage form at rates that are proportional relative to the respective weights of each active agent in the dosage form.

75. The sustained release dosage form of claim 39, wherein the pharmaceutically active agents are released from the dosage form at rates that are proportional relative to the respective weights of each active agent in the dosage form.
